(19) United States
(12) Patent Application Publication
(10) Pub. No.: US 2026/0226506 A1
(43) Pub. Date: Aug. 6, 2026

(54) METHODS OF ENHANCING DIVERSITY OF HLA HAPLOTYPE EXPRESSION IN TUMORS TO BROADEN TUMOR CELL SUSCEPTIBILITY TO TCR-T THERAPY

(71) Applicants: T-Cure Bioscience, Inc., Irvine, CA (US); Genvira Biosciences, Inc., Ottawa (CA)

(72) Inventors: Gordon PARRY, Irvine, CA (US); Lijuan FU, Irvine, CA (US); Gang ZENG, Irvine, CA (US); Nicholas DAVIS, Irvine, CA (US); Erika Von Euw, Irvine, CA (US); Jiahu Wang, Ottawa (CA)

(21) Appl. No.: 19/391,755

(22) Filed: Nov. 17, 2025

Related U.S. Application Data
(60) Provisional application No. 63/722,020, filed on Nov. 18, 2024.
(60) Provisional application No. 63/816,533, filed on Jun. 2, 2025.

(51) Int. Cl. C12N 15/86; A61K 31/7088; A61K 35/17; A61K 40/11; A61K 40/32; A61K 40/42; A61K 48/00; C07K 14/705; C12N 5/09; C12N 15/10; C12N 15/11

(52) U.S. Cl. CPC C12N 15/86; A61K 31/7088; A61K 35/17; A61K 40/11; A61K 40/32; A61K 40/42; A61K 48/00; C07K 14/70539; C12N 5/0693; C12N 15/102; C12N 15/11; C12N 2510/00

(57) ABSTRACT

The present invention provides methods for increasing the sensitivity of tumor cells to a TCR-engineered T cells (TCR-T) therapy comprising genetically modifying the tumor cells to express an haplotype, for example an HLA haplotype, different from the haplotype endogenous to the tumor cells.

### CROSS-REFERENCE TO RELATED APPLICATIONS

This application claims the benefit of U.S. Provisional Application No. 63/816,533, filed Jun. 2, 2025, and U.S. Provisional Application No. 63/722,020, filed Nov. 18, 2024, all of which are incorporated herein by reference in their entireties.

### SEQUENCE LISTING

The instant application contains a Sequence Listing submitted electronically in XML format in accordance with WIPO Standard ST.26, which is hereby incorporated by reference in its entirety. The XML file, created on Feb. 17, 2026, is named 128882-5005-US Sequence Listing.xml and is 57,552 bytes in size.

### BACKGROUND OF THE INVENTION

TCR-T represents a promising approach for immunotherapy of solid tumors. It has been known since the late 1980s that adoptive transfer of tumor infiltrating lymphocytes (TIL) was able to generate objective tumor regression in melanoma and kidney cancer patients (Rosenberg SA. 2001. Nature 411: 380-4). Molecular cloning of tumor-associated antigens was carried out in the 1990s mainly for melanoma and resulted in the identification of melanoma/melanocyte differentiation antigen MART-1, gp100, and tyrosinase. Additionally, shared cancer/testis antigens NY-ESO-1, MAGE-A3, MAGE-A4, KK-LC-1, PRAME, and SSX-2, as well as tumor-specific antigens such as mutated p53, mutated KRAS, HERV-E, HPV-E6 and so on, were identified as the molecular targets recognized by TILs (1). Subsequent to TIL based therapy, adoptive transfer of T cells, in most cases CD8⁺ T cells, engineered to express TCR's specifically targeting these tumor-associated antigens (TAA) have achieved certain success in selected melanoma patients (Rosenberg SA. 2014. Nat Rev Clin Oncol 11: 630-2; Dudley M E, et al., 2001. J Immunother 24: 363-73; and Dudley M E, et al., 2002. Science 298: 850-4). In particular, one case report showed the use of an HLA-DP4-restricted CD4⁺ T cell clone against NY-ESO-1 gave rise to complete responses mediated by a mechanism called epitope spreading (Hunder N N, et al., 2008. N Engl J Med 358: 2698-703). Consequently, scientists have been using gene transfer to introduce TCRs to make T cells tumor reactive (Morgan R A, Dudley M E, et al., 2006. Science 314: 126-9). Adoptive transfer of gene-engineered MART-1 TCR clinical trial was carried out in 2006 and 2 out of 17 (12%) patients with metastatic melanoma experienced anti-tumor responses, which although far from a cure and lower than the rate observed for TIL, provided proof-of-concept that gene-engineered peripheral T cells could exhibit anti-tumor activity in patients with advanced metastatic melanoma (Morgan R A, et al., 2006. Science 314: 126-9). The first clinical study to treat patients beyond melanoma used retrovirus to deliver a NY-ESO-1 TCR to T cells, was published in 2011 (Robbins P F, et al., 2011. J Clin Oncol 29: 917-24). It was then followed in 2015 by another study using a TCR targeting the same epitope of NY-ESO-1 (Robbins P F, et al., 2015. Clin Cancer Res 21: 1019-27). Objective clinical responses were seen in 11/18 (61%) patients with synovial cell sarcoma, and 11/20 (55%) with melanoma (Robbins P F, et al., 2015. Clin Cancer Res 21: 1019-27). Both groups of patients had failed previous chemo- and radiation therapy.

The latest data on NY-ESO-1 was published in 2019 (Blood Adv. 2019 Jul. 9; 3(13): 2022-2034.) In this study 25 patients received an infusion of up to 1×10¹⁰ NY-ESO-1 specific peptide enhanced affinity receptor (SPEAR) T cells. Objective response rate was 80% at day 42; 76% at day 100 and 44% at 1 year. At year 1, 13/25 patients were disease progression-free (52%); 11 were responders (1 stringent complete response, 1 complete response, 8 very good partial response, 1 partial response).

Engineered T cell receptor therapy involves treating cancer with activated T lymphocytes from the body, similarly to CAR-T therapies. Both strategies attach new receptors to the cells' surfaces, enabling them to attack different forms of cancer. The distinction between the two methods pertains to what antigens they are capable of recognizing. CAR-T cells bind to naturally occurring antigens on the surface of cancer cells, whereas TCR-based therapeutic approaches can access intracellular antigens. Since the vast majority of TAA are intracellular proteins, they are only accessible by T cell receptor (TCR)-based but not chimeric antigen receptor (CAR)-based therapeutic approaches. Therefore, TCR-T and TCR-CD3 bispecific antibodies are important approaches to target TAA. With engineered TCR therapy (TCR-T), the added receptors can only link with MHC proteins. As such, there remains a need in the art to allow for broader applicability of TCR-T to cancer types by altering the haplotype to allow for TCR-T.

Recognition of an antigenic epitope and HLA complex by T-cell receptors (TCRs) is the natural surveillance mechanism for T cells to eliminate endogenously arising tumor cells. TCR-engineered T cells are now used in adoptive cell transfer therapy against various tumor types with significant success in the clinic.

However, in many circumstances, a patient is ineligible to be treated by TCR-T therapy due to the absence of a matching HLA that is needed for the TCR to recognize the peptide on the surface of tumor cells. In order to address this limitation, this example provides methods for an approach that will allow patients to be eligible for TCR-T therapy even in the absence of a matched HLA haplotype. This example provides a technology based on engineering a patient's tumor cells to specifically express the required HLA that matches the selected TCR. When this method is combined with a tumor selective gene delivery approach, minimal toxicity is predicted due to the fact that only the tumors cells and not normal tissues will express both target and required haplotype. In addition, the approach may also address the issue of downregulation of HLA by tumor cells that limits the success of TCR-T therapy in autologous settings.

The requirement that tumors express both the tumor associated antigen (TAA) and a matched HLA haplotype for effective TCR based killing limits the size of the population that can be treated with TCR based therapy: Indeed, discovery of effective TCRs that target shared TAAs is one of the major bottlenecks in the immunotherapy of solid tumors (Cole D J, et al., Cancer Res 54: 5265-8, Clay T M, et al., 1999. J Immunol 163: 507-13). So far, the majority of the TCRs that have been successful in the clinic are HLA-A2-restricted, and NY-ESO-1 has been the most successful TAA in early clinical trials on metastatic melanoma, synovial cell sarcoma and multiple myeloma (Robbins P F, et al., 2011. J Cin Oncol 29: 917-24, and Robbins P F, et al., 2015. Clin Cancer Res 21: 1019-27, and Rapoport A P, et al., 2015. Nat Med). Theoretically, we can think of two approaches for making a NY-ESO-1 HLA-A2-specific TCR applicable to otherwise non-compatible patients. One is through introducing NY-ESO-1 antigen into NY-ESO-1 null tumors in HLA-A2 patients; the other is introducing HLA-A2 into NY-ESO-1 positive tumors for non-HLA-A2 patients. In practice, to engineer tumor specific expression of a TAA in HLA-matched patient may pose potential toxicities due to off-target expression of TAA in somatic cells. In contrast, forced tumor specific expression of HLA in otherwise HLA-mismatched patients has less probability of off-target toxicity as such toxicity requires the expression of both TAA and the mismatched HLA. Strategies of acquired cytotoxicity by engineering patient tumor cells to express an otherwise allogeneic HLA may represent a new avenue in cancer immunotherapy.

Kimmtrak® (tebentafusp-tebn) is the only FDA-approved therapy which utilizes TCR-CD3 against the HLA-A2-restricted gp100 epitope for uveal melanoma.⁵ The TCR-T field has received significant attention over the last few years and there are now a few late stage clinical trials, such as Afami-cel targeting the HLA-A2-restricted MAGE-A4 epitope for synovial cell sarcoma, Lete-cel targeting the HLA-A2-restricted NY-ESO-1 epitope for synovial cell sarcoma and MRCLS, and IMA203 targeting the HLA-A2-restricted PRAME epitope for melanoma, synovial cell sarcoma, head and neck cancer, and ovarian cancers.

These trials have served to highlight the significant roadblocks facing these emerging immunotherapies against late-stage solid tumors which include limits due to HLA restrictions and lack of in situ activation and expansion of TCR-T.

### Limitation by HLA Restrictions

Patients must possess the HLA-A*0201 haplotype with the exclusion of the HLA-A*0205 haplotype for eligibility of Afami-cel. Eligible patients for Lete-Cel need to express NY-ESO-1 and/or LAGE-1a and either HLA-A*02:01, *02:05, or *02:06.⁷ In the synovial cell sarcoma patient population, only a small proportion of them, about 30%, express MAGE-A4, and are thus eligible. Strict HLA requirements for TCR-based therapies also means that loss of the specific HLA or β-2 microglobulin on the tumor-cell surface in vivo represents a challenging condition for successful T cell-based cancer treatments.

### Lack of In Situ Activation and Expansion of TCR-T

Current TCR-T approaches require the patient to undergo chemotherapy for lymophodepletion to allow expansion and survival of adoptively transferred TCR-T in vivo. Even with this tedious pre-conditioning, patients are often given 10¹⁰ TCR-T to achieve proper efficacy. Requirement of lymphodepletion and huge numbers of activated TCR-T have contributed to the high cost of such therapies. The often-exhausted conditions of infused T cells and the subsequent lack of activation and expansion at the tumor site are widely believed to be a factor for limited clinical success.

Oncolytic Virus-Based Therapies for Solid Tumors with Potential Synergistic Effects Combining with TCR-T.

Oncolytic viruses are recombinant viruses that have been engineered to infect cancer cells. Originally conceived as cancer cell killers on their own, advances in backbone engineering have seen their repurposing to assist in other anti-cancer modalities. The Western Reserve Strain oncolytic vaccinia virus has been a common tool for this purpose. Multiple modifications of this vaccinia strain have been made and tested for use as gene therapy vectors. Common modifications include the deletion of viral genes for growth and replication in normal cells as well as the addition of multiple payloads. These modifications make the virus potent oncolytically but safe for normal cells and thus make vaccinia an attractive platform for gene delivery. Using a strain of vaccinia that has both viral growth factor (VGF) and Thymidine Kinase (TK) deleted has shown to create a virus (vvDD) that has powerful oncolytic effects with little activity towards normal cells as well as the capacity to be carry other genes that can then be expressed in infected cells. In addition to the functions of the specific payloads, oncolytic viruses often exert generic functions through disrupting tumor vascular structures, and direct tumor killings through the oncolytic function.

So far, the FDA has only approved T-VEC, a herpes simplex virus type-1 designed to selectively replicate within tumors and to produce GM-CSF to enhance systemic antitumor immune responses for metastatic melanoma. However, several late-stage therapies are drawing attention in recent clinical trials, for example, a herpes virus encoding TGF-β and adenovirus encoding GM-CSF (cretostimogene grenadenorepvec) for bladder cancer.

The present invention meets these present needs by providing methods for increasing the sensitivity of tumor cells to a TCR-engineered T cells (TCR-T) therapy comprising genetically modifying the tumor cells to express an haplotype, for example an HLA haplotype, different from the haplotype endogenous to the tumor cells, through a novel class of oncolytic virus-induced HLA-Independent TCR-T (iHITT) therapies.

### BRIEF SUMMARY OF THE INVENTION

The present invention provides a method for increasing the sensitivity of a population of tumor cells to a TCR-engineered T cell (TCR-T) therapy, the method comprising genetically modifying the population of tumor cells to express a tumor haplotype different from the tumor haplotype endogenous to the population of tumor cells.

The present invention also provides a method of upregulating antigen presentation on the cellular surfaces of a population of tumor cells to increase sensitivity of the population of tumor cells to a TCR-engineered T cell (TCR-T) therapy comprising genetically modifying the population of tumor cells to express a tumor haplotype different from the tumor haplotype endogenous to the population of tumor cells.

The present invention also provides a method of reversing downregulation of expression of a tumor haplotype gene in a population of tumor cells in order to increase sensitivity of the population of tumor cells to a TCR-engineered T cell (TCR-T) therapy, wherein the method comprises genetically modifying the population of tumor cells to express the tumor haplotype.

The present invention also provides a method for increasing HLA expression to render a population of tumor cells susceptible to autologous T cells, wherein the method comprises genetically modifying the population of tumor cells to express the HLA haplotype.

In some embodiments, the method further comprises expressing the tumor haplotype that is different from the tumor haplotype that is endogenous to the population of tumor cells.

In some embodiments of the method, the methods include expressing the tumor haplotype that is different from the tumor haplotype that is endogenous to the population of tumor cells allows for targeting the population of tumor cells with the TCR-T.

The present invention also provides a method for increasing the sensitivity of a tumor cell to a TCR-engineered T cell (TCR-T) therapy comprising:
a) determining the tumor haplotype of the population of tumor cells;
b) contacting the population of tumor cells with a nucleic acid encoding a tumor haplotype different from the tumor haplotype endogenous to the tumor cells, wherein the tumor haplotype different from the tumor haplotype endogenous to the tumor cells is expressed, and wherein the population of tumor cells exhibit increased sensitivity to a TCR-T therapy.

In some embodiments, the tumor haplotype different from the tumor haplotype endogenous to the tumor cells is expressed and upregulates antigen presentation.

In some embodiments, the tumor haplotype different from the tumor haplotype endogenous to the tumor cells is expressed and reverses downregulation of expression of a tumor haplotype gene.

The present invention also provides a method for increasing HLA expression to render a population of tumor cells susceptible to a TCR-engineered T cell (TCR-T) therapy comprising:
a) determining the HLA haplotype of the population of tumor cells;
b) contacting the population of tumor cells with a nucleic acid encoding an HLA haplotype different from the HLA haplotype endogenous to the tumor cells, wherein the HLA haplotype different from the HLA haplotype endogenous to the tumor cells is expressed, and wherein the population of tumor cells exhibit increased sensitivity to a TCR-T therapy.

In some embodiments, the method comprises contacting the population of tumor cell with a nucleic acid encoding the tumor haplotype that is different from the tumor haplotype endogenous to the population of tumor cells.

In some embodiments, the method comprises contacting the population of tumor cells with a vector encoding the tumor haplotype that is different from the tumor haplotype endogenous to the population of tumor cells.

In some embodiments, the nucleic acid or vector is introduced and/or integrated into the population of tumor cells such that there is stable expression of the tumor haplotype that is different from the tumor haplotype endogenous to the population of tumor cells.

In some embodiments, the nucleic acid or vector is stably integrated into the genome of the population of tumor cells.

In some embodiments, the nucleic acid or vector is introduced and/or integrated into at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells such that there is stable expression of the tumor haplotype encoded by the nucleic acid or vector.

In some embodiments, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells stably express the tumor haplotype that is different from the tumor haplotype endogenous to the population of tumor cells.

The present invention provides for the use of a vector in a method for increasing the sensitivity of a population of tumor cells to a TCR-engineered T cell (TCR-T) therapy comprising genetically modifying the population of tumor cells to express a tumor haplotype different from the tumor haplotype endogenous to the tumor cells.

The present invention provides for the use of a vector in a method of upregulating antigen presentation on the cellular surfaces of a population of tumor cells to increase sensitivity of the population of tumor cells to a TCR-engineered T cell (TCR-T) therapy comprising genetically modifying the population of tumor cells to express a tumor haplotype different from the tumor haplotype endogenous to the tumor cells.

The present invention provides for the use of a vector in a method of reversing downregulation of expression of a tumor haplotype gene in a population of tumor cells in order to increase sensitivity of the population of tumor cells to a TCR-engineered T cell (TCR-T) therapy, wherein the method comprises genetically modifying the population of tumor cells to express the tumor haplotype.

The present invention provides for the use of a vector in a method for increasing HLA expression to render a population of tumor cells susceptible to allogeneic T cells, wherein the method comprises genetically modifying the population of tumor cells to express the HLA haplotype.

The present invention provides for the use of a vector in a method for increasing HLA expression to render a population of tumor cells susceptible to autologous T cells, wherein the method comprises genetically modifying the population of tumor cells to express the HLA haplotype.

In some embodiments, the vector is a non-viral vector or viral vector.

In some embodiments, the vector is administered to a subject in need thereof systemically, intratumorally, and/or intravenously.

In some embodiments, the vector is viral vector.

In some embodiments, the viral vector is selected from the group consisting of a vaccinia (pox) virus vector, herpes simplex virus vector, myxoma virus, coxsackie virus vector, poliovirus vector, Newcastle disease virus vector, retrovirus vector (including lentivirus vector or a pseudotyped vector), an adenovirus vector, an adeno-associated virus vector, a simian virus vector, a sendai virus vector, measles virus vector, foam virus vector, alphavirus vector, and vesicular stomatitis virus vector.

In some embodiments, the viral vector is selected from the group consisting of a vaccinia (pox) virus vector, herpes simplex virus vector, and myxoma virus.

In some embodiments, the viral vector is a vaccinia (pox) virus vector and the administration route is systemic.

In some embodiments, the viral vector is a herpes simplex virus vector and the administration route is intratumoral.

In some embodiments, the viral vector is a myxoma virus and the administration route is systemic.

In some embodiments, the TCR-T is administered subsequently to genetically modifying the population of tumor cells to express a tumor haplotype different from the tumor haplotype endogenous to the population of tumor cells.

In some embodiments, the tumor haplotype different from the tumor haplotype endogenous to the population of tumor cells is a HLA, NY-ESO, HERV, LAGE, MAGE, MUC, BAGE, RAGE, CAGE, SSX, PRAME, PSMA, XAGE, tyrosinase, melan-A, mutated kRAS, mutated P53, or HPV E5 and E6 tumor haplotype.

In some embodiments, the tumor haplotype different from the tumor haplotype endogenous to the population of tumor cells is a HLA-A, HLA-A2, HLA-A3, HLA-B, HLA-C, HLA-G, HLA-E, HLA-F, HLA-DPA1, HLA-DQA1, HLA-DQB1, HLA-DQB2, HLA-DRB1, HLA-DRB5, KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6 tumor haplotype.

In some embodiments, the tumor haplotype different from the tumor haplotype endogenous to the population of tumor cells is a HLA, HLA-A2, KK-LC-1, NY-ESO-1, or HERV-E tumor haplotype.

In some embodiments, the HLA haplotype is selected from the group consisting of HLA-A, HLA-A2, HLA-A3, HLA-B, HLA-C, HLA-G, HLA-E, HLA-F, HLA-DPA1, HLA-DQA1, HLA-DQB1, HLA-DQB2, HLA-DRB1, and HLA-DRB5.

In some embodiments, the HLA haplotype is HLA-A2.

In some embodiments, the HLA haplotype is an MHC class I haplotype.

In some embodiments, the tumor haplotype different from the tumor haplotype endogenous to the population of tumor cells is an HLA tumor haplotype, and wherein the TCR-T comprises an HLA restricted and/or targeted TCR.

In some embodiments, the tumor haplotype different from the tumor haplotype endogenous to the population of tumor cells is an HLA tumor haplotype, and wherein the TCR-T comprises a restricted and/or targeted TCR, wherein the restricted and/or targeted TCR-T binds to KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

In some embodiments, the tumor haplotype different from the tumor haplotype endogenous to the population of tumor cells is a HLA tumor haplotype, and wherein the TCR-T comprises a KK-LC-1 restricted and/or targeted TCR.

In some embodiments, the tumor haplotype different from the tumor haplotype endogenous to the population of tumor cells is a HLA tumor haplotype, and wherein the TCR-T comprises an HERV-E restricted and/or targeted TCR.

In some embodiments, the tumor haplotype different from the tumor haplotype endogenous to the population of tumor cells is a HLA tumor haplotype, and wherein the TCR-T comprises an NY-ESO-1 restricted and/or targeted TCR.

In some embodiments, the tumor haplotype endogenous to the population of tumor cells is a null haplotype or the absence of the tumor haplotype.

In some embodiments, the population of tumor cells are from a solid tumor.

In some embodiments, the solid tumor is selected from the group consisting of sarcoma, carcinoma, and lymphoma.

In some embodiments, the solid tumor is from a cancer or carcinoma of the bladder, uterine cervix, stomach, breast, lung, colon, rectum, skin, melanoma, gastrointestinal tract, urinary tract, or pancreas.

In some embodiments, the tumor cells are in vitro.

In some embodiments, the tumor cells are in vivo.

In some embodiments, the method or use is for the treatment of cancer in a subject in need thereof.

In some embodiments, administration of the TCR-T inhibits solid tumor growth.

In some embodiments, the TCR-T comprises TCR-T cells, including an infusion of TCR-T cells.

In some embodiments, the TCR-T therapy comprises a TCR having antigenic specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

In some embodiments, the TCR-T therapy comprises a TCR having antigenic specificity for HERV-E, KK-LC-1, or NY-ESO-1.

In some embodiments, the TCR-T therapy comprises a TCR having antigenic specificity for Kita-Kyushu Lung Cancer Antigen-1₅₂₋₆₀ (KK-LC-1₅₂₋₆₀).

In some embodiments, the KK-LC-1₅₂₋₆₀ comprises the amino acid sequence NTDNNLAVY (SEQ ID NO:11).

In some embodiments, the TCR-T therapy comprises a TCR having antigenic specificity for HERV-E.

In some embodiments, the HERV-E comprises the amino acid sequence ATFLGSLTWK (SEQ ID NO:22).

In some embodiments, the TCR-T therapy comprises a TCR having antigenic specificity for NY-ESO-1157-165.

In some embodiments, the NY-ESO-1157-165 comprises the amino acid sequence SLLMWITQC (SEQ ID NO:33).

In some embodiments, the TCR comprises the amino acid sequences of SEQ ID NO: 5 and/or 10.

In some embodiments, the TCR comprises the amino acid sequences of SEQ ID NO: 16 and/or 21.

In some embodiments, the TCR comprises the amino acid sequences of SEQ ID NO: 27 and/or 32.

In some embodiments, the TCR comprises the amino acid sequences of SEQ ID NO: 38 and/or 43.

In some embodiments, the TCR comprises nucleic acids encoding a TCR beta chain and a TCR alpha chain, wherein the nucleotide sequence encoding the beta chain is positioned 5′ of the nucleotide sequence encoding the alpha chain.

In some embodiments, the TCR-T comprises a T-cell receptor α-chain comprising an amino acid sequence encoded by a nucleic acid sequence comprising an αCDR1, αCDR2, and αCDR3; a T-cell receptor β-chain comprising an amino acid sequence encoded by a nucleic acid sequence comprising βCDR1, βCDR2, and βCDR3; or both.

In some embodiments, the TCR-T comprises a T-cell receptor α-chain comprising an amino acid sequence encoded by a nucleic acid sequence comprising at least 90% sequence identity to the nucleic acid sequence of SEQ ID NO: 6, 17, 28 or 39; a T cell receptor β-chain comprising an amino acid sequence encoded by a nucleic acid sequence comprising at least 90% sequence identity to the nucleic acid sequence of SEQ ID NO: 1, 12, 23, or 34; or both.

In some embodiments, the TCR-T comprises a T-cell receptor α-chain comprising an amino acid sequence encoded by a nucleic acid sequence comprising the αCDR1, αCDR2, and αCDR3 from a sequence selected from the group consisting of SEQ ID NO: 6, 17, 28 or 39; a T cell receptor β-chain comprising an amino acid sequence encoded by a nucleic acid sequence comprising the βCDR1, βCDR2, and βCDR3 from a sequence selected from the group consisting of SEQ ID NO: 1, 12, 23, or 34; or both.

In some embodiments, the TCR-T comprises a T-cell receptor α-chain comprises the αCDR1, αCDR2, and αCDR3 from a sequence selected from the group consisting of SEQ ID NO: 5, 16, 27, or 38; a T-cell receptor β-chain comprises the βCDR1, βCDR2, and βCDR3 from a sequence selected from the group consisting of SEQ ID NO: 10, 21, 32, or 43; or both.

In some embodiments, the TCR-T comprises a T-cell receptor α-chain comprising an amino acid sequence encoded by a nucleic acid sequence of SEQ ID NO: 5, 16, 27, or 38; a T-cell receptor β-chain comprising an amino acid sequence encoded by a nucleic acid sequence of SEQ ID NO: 10, 21, 32, or 43; or both.

In some embodiments, the vector comprises the nucleic acid sequence of SEQ ID NO: 1 and the nucleic acid sequence of SEQ ID NO: 6, or comprises a nucleic acid encoding for the amino sequence of SEQ ID NO: 5 and a nucleic acid encoding for the amino sequence of SEQ ID NO: 10.

In some embodiments, the vector comprises the nucleic acid sequence of SEQ ID NO: 12 and the nucleic acid sequence of SEQ ID NO: 17, or comprises a nucleic acid encoding for the amino sequence of SEQ ID NO: 16 and a nucleic acid encoding for the amino sequence of SEQ ID NO: 21.

In some embodiments, the vector comprises the nucleic acid sequence of SEQ ID NO: 23 and the nucleic acid sequence of SEQ ID NO: 28, or comprises a nucleic acid encoding for the amino sequence of SEQ ID NO: 27 and a nucleic acid encoding for the amino sequence of SEQ ID NO: 32.

In some embodiments, the vector comprises the nucleic acid sequence of SEQ ID NO: 34 and the nucleic acid sequence of SEQ ID NO: 39, or comprises a nucleic acid encoding for the amino sequence of SEQ ID NO: 38 and a nucleic acid encoding for the amino sequence of SEQ ID NO: 43.

In some embodiments, the vector comprises a nucleic acid sequence encoding the βCDR1, βCDR2, and βCDR3 of SEQ ID NO: 1 and a nucleic acid sequence encoding the αCDR1, αCDR2, and αCDR3 of SEQ ID NO: 6.

In some embodiments, the vector comprises a nucleic acid sequence encoding the βCDR1, βCDR2, and βCDR3 of SEQ ID NO: 12 and a nucleic acid sequence encoding the αCDR1, αCDR2, and αCDR3 of SEQ ID NO: 17.

In some embodiments, the vector comprises a nucleic acid sequence encoding the βCDR1, βCDR2, and βCDR3 of SEQ ID NO: 23 and a nucleic acid sequence encoding the αCDR1, αCDR2, and αCDR3 of SEQ ID NO: 28.

In some embodiments, the vector comprises a nucleic acid sequence encoding the βCDR1, βCDR2, and βCDR3 of SEQ ID NO: 34 and a nucleic acid sequence encoding the αCDR1, αCDR2, and αCDR3 of SEQ ID NO: 39.In some embodiments, the method or use according to any of the previous claims, wherein the method comprises genetically modifying the population of tumor cells to increase expression of a chemokine at the tumor site.

In some embodiments, the increased expression of a chemokine at the tumor site leads to increased presence or activity of T cells at the tumor site.

In some embodiments, the chemokine is at least one of CXCL9, CXCL10, and CXCL11.

In some embodiments, the method comprises genetically modifying a tumor cell in the population of tumor cells to increase expression of CD80 on the tumor cell surface.

The present invention also provides a peptide comprising the amino acid sequence NTDNNLAVY (SEQ ID NO:11).

The present invention also provides a peptide comprising the amino acid sequence ATFLGSLTWK (SEQ ID NO:22).

The present invention also provides a peptide comprising the amino acid sequence SLLMWITQC (SEQ ID NO:33).

In some embodiments, the TCR-T therapy comprises a TCR having antigenic specificity for a peptide selected from the group consisting of NTDNNLAVY (SEQ ID NO:11), ATFLGSLTWK (SEQ ID NO:22), and SLLMWITQC (SEQ ID NO:33).

### BRIEF DESCRIPTION OF THE DRAWINGS

FIG. 1: HERV-E expression: qPCR data for HERV-E expression in COLO-205 (colon), SK-LU-1 (lung), FM-6 (skin), A498 (clear cell kidney) and 1755R (clear cell kidney). All data are normalized by copies per 10⁵ beta actin.

FIG. 2: Transduction of HERV-E-TCR: Representative transduction of T cells stained with anti-CD34 in untransduced and HERV-E-TCR transduced cells.

FIG. 3: Co-Culture of donor HERV-E-TCR T cells and A*11 transduced target cells: Donor T cells (donors 389, 601, and 801) were transduced with HERV-E-TCR and co-cultured with A498, A498+A*11, 1755R, and 1755R+A*11.

FIG. 4: T cell transductions: Unstained (US), Untransduced (UT), and 5 donor T cells transduced with KK-LC-1-TCR. UT and donor cells stained with anti-mouse-TCR-Beta (BV421). Donors T cells are 199, 200, 397, 511, and 512.

FIG. 5: Expression of KK-LC-1 (CT83) in Normal and Tumor Cells. Normal cells RNA from pools of 5 donors (testis, brain, and lung). All expression levels relative to beta-actin.

FIGS. 6A-D: Interferon-gamma release upon coculture with KK-LC-1-TCR transduced T cells in DU-145 (FIG. 6A) and MKN-45 (FIG. 6B), and normalized tumor volumes post-injection after female NSG mice were injected with MKN-45 (FIG. 6C) and MKN-45-A*01 (FIG. 6D) tumors on each flank subcutaneously. Donor T cells are denoted by donor number and “R” for retroviral transduction (199R, 200R, 397R, 511R, and 512R).

FIG. 7: T cell transductions: Unstained (US), Untransduced (UT), and 5 donor T cells. UT and donor cells stained with anti-mouse-TCR-Beta (BV421). Donors T cells are donor numbers 199 and 200.

FIGS. 8A-B: Interferon-gamma release upon coculture with NY-ESO-1-TCR transduced T cells in donor 199 (FIG. 8A) and donor 200 (FIG. 8B). Target cells are denoted by type and A*02 status.

FIG. 9: Plasmid Map of the vvDD-P2600 Insert. Construct of vvDD-P2600 vaccinia virus encoding a iHITT cassette in the context of HLA-A*0101. A similar construct encoding the HLA-A*0201, β2-microglobulin (B2M), and CD80, is named vvDD-P2700 for this study. A similar construct encoding the HLA-A*1101, β2-microglobulin (B2M), and CD80, is named vvDD-P2800 for this study.

FIG. 10: Proposed mechanism of action for vvDD-P2600 encoding HLA-A*0101 (HLA-A-1), B2M, and CD80 for inducing activation, expansion of 820 KK-LC-1 TCR-T as well as tumor killings without matching HLA in vivo. A similar mechanism of action is proposed for vvDD-2700 and vvDD-P2800.

FIGS. 11A-B: (FIG. 11A) Preliminary data showing induced HLA-independent 820 KK-LC-1 TCR-T killing, and (FIG. 11B) recognition of non-HLA-A1 tumors indicated by IFN-7 secretion. Percentages of tumor killings in FIG. 11A were estimated based on the number of tumor cells alone without 820 TCR-T co-culturing, as the baseline. vvDD was the parental virus used as the vector control for FIG. 11A and FIG. 111B.

FIG. 12: Scheme of treatment model using human triple negative breast cancer (TNBC) line MDA-MB-468 in female immunodeficient (e.g., NSG) mice. Following tumor establishment, vvDD-P2600 will be injected intratumorally (IT) with subsequent TCR-T infusion through tail vein (IV). Primary endpoints will be tumor burdens and animal survival over time; secondary endpoints will be tumor infection rate by vvDD-P2600, expansion and longevity of infused 820 TCR-T in vivo.

FIGS. 13A-L: Quality control assessment of 820TCR-T cells expanded for 14 days. FIG. 13A shows the population of CD4+/8+ cells, and FIG. 13B shows the percentage of 820TCR positive T cells. FIG. 13C shows GM-CSF secretion measured as pg/mL from CD4+, CD8+, and total 820TCR-T cells. FIG. 13D shows the percentage of intracellular IFNγ positive CD4+ and CD8+ 820TCR-T cells. The flow cytometry data used to generate the graph and table shown in FIG. 13D is shown in FIGS. 13E to 13L.

FIG. 14: Schematic of study used in FIGS. 15-17.

FIG. 15: Measurement of tumor volume (mm³) over time in mice when implanted MDA-MB-468 (KK-LC-1+/HLA-A1−) triple negative breast cancer tumors were injected with vvDD-P2600 (OV101) on day 24 and then 820 TCR-T cells on day 28 after implantation (solid line with standard deviations indicated). Growth of an untreated tumor is shown for comparison (solid dots as measured timepoints and curve fitting shown as a dotted line).

FIG. 16: Measurement of the oncolytic virus' infection efficiency and the infiltration of T cells in the tumour tissue post-Treatment for up to 42 days.

FIG. 17: The percentage of TCR-T cells in the isolated splenocytes of the control and treatment groups.

FIG. 18: Schematic of an exemplary treatment regimen in human patients.

FIGS. 19A-B: KK-LC-1 TCR sequence information.

FIGS. 20A-B: HERV-E TCR sequence information.

FIGS. 21A-B: NY-ESO-1 TCR sequence information.

FIGS. 22A-B: 1G4-LY-TCR TCR sequence information.

FIG. 23: Human HLA-A*0101 sequence information (SEQ ID NO: 45).

FIGS. 24A-B: FIG. 24A shows human HLA-A*0201 sequence information. (SEQ ID NO: 46). FIG. 24B shows human HLA-A*1101 sequence information (SEQ ID NO. 49).

FIG. 25: Human β2-microglobulin (B2M) sequence information. (SEQ ID NO: 47).

FIG. 26: Human CD80 cDNA sequence information. (SEQ ID NO: 48).

FIGS. 27A-C: Infection efficiency of vvDD-P2600-mCherry (OV101) in two unstained tumor cell lines (FIG. 27A). Expression levels of HLA-A01 (green) and CD80 (yellow) in infected A375 and U2OS cells stained with antibodies (FIG. 27B). Percentage of CD80+ A549 cells at MOI=0.1, 1, 10 (FIG. 27C). Untreated cells serve as the control.

### DETAILED DESCRIPTION OF THE INVENTION

### I. Introduction

Recognition of an antigenic epitope and HLA complex by T-cell receptors (TCRs) is the natural surveillance mechanism for T cells to eliminate endogenously arising tumor cells. TCR-engineered T cells are now used in adoptive cell transfer therapy against various tumor types with significant success in the clinic. However, in many circumstances, a patient is ineligible to be treated by TCR-T therapy due to the absence of a matching HLA that is needed for the TCR to recognize the peptide on the surface of tumor cells. In order to address this limitation, the present invention provides an approach that will allow patients to be eligible for TCR-T therapy even if they don't have a matched haplotype. The technology described in the present invention is based on engineering a patient's tumor cells to specifically express the required HLA that matches the selected TCR. When combined with a tumor selective gene delivery approach, minimal toxicity is predicted due to the fact that only the tumors cells and not normal tissues will express both target and required haplotype. In addition, the approach may also address the issue of downregulation of HLA by tumor cells that limits the success of TCR-T therapy in autologous settings.

### I. Selected Definitions

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements.

Unless explained otherwise, all technical and scientific terms used herein have the same meaning as commonly understood to one of ordinary skill in the art to which this disclosure belongs.

As used herein, “contact” or “contacting” refers to the relatively close physical proximity of one object to another object. Generally, contacting involves placing two or more objects in close physical proximity to each other to give the objects and opportunity to interact. For example, contacting a population of tumor cells with a nucleic acid or vector can be accomplished by placing the nucleic acid or vector in physical proximity to the population of tumor cells, for example by injecting the nucleic acid or vector into a subject or patient having the solid cancer. Similarly, in vitro contact can also occur, for example by adding the nucleic acid or vector into culture media in which the population of tumor cells is growing.

As used herein, “TCR complex” or “TCR” generally refers to a complex formed by the association of CD3 with a TCR. For example, a TCR complex can be composed of a CD3y chain, a CD3P chain, two CD3s chains, a homodimer of CD3ζ chains, a TCRa chain, and a TCRP chain. Alternatively, a TCR complex can be composed of a CD3y chain, a CD3P chain, two CD3s chains, a homodimer of CD3C, chains, a TCRy chain, and a TCRP chain. In some instances, a “component of a TCR complex”, as used herein can refer to a TCR chain (for example, TCRa, TCRp, TCRy or TCR5), a CD3 chain (for example, CD3y, CD35, CD3s or CD35, or a complex formed by two or more TCR chains or CD3 chains (for example, a complex of TCRa and TCRP, a complex of TCRy and TCR5, a complex of CD3s and CD35, a complex of CD3y and CD3s, or a sub-TCR complex of TCRa, TCRp, CD3y, CD35, and two CD3s chains).

Principles of antigen processing by antigen presenting cells (APC) (such as dendritic cells, macrophages, lymphocytes or other cell types), and of antigen presentation by APC to T cells, including major histocompatibility complex (MHC)-restricted presentation between immunocompatible (for example, sharing at least one allelic form of an MHC gene that is relevant for antigen presentation) APC and T cells, are well established (see, e.g., Murphy, Janeway's Immunobiology (8th Ed.) 201 1 Garland Science, NY; chapters 6, 9 and 16). For example, processed antigen peptides originating in the cytosol are generally from about 7 amino acids to about 11 amino acids in length and will associate with class I MHC (HLA) molecules.

As used herein, an “altered domain” or “altered protein” or “substituted domain” or “substituted protein” refers to a motif, region, domain, peptide, polypeptide, or protein with a non-identical sequence identity to a wild-type or reference motif, region, domain, peptide, polypeptide, or protein (for example, a wild type TCRa chain, TCRP chain, TCRa constant domain, TCRP constant domain) of at least 85% (e.g., 86%, 87%, 88%, 89%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%). Altered domains or altered proteins or derivatives can include those based on all possible codon choices for the same amino acid and codon choices based on conservative amino acid substitutions. Substitutional analogs typically exchange one amino acid of the wild-type or reference sequence for another at one or more sites within the protein, and may be designed to modulate one or more properties of the polypeptide without the complete loss of other functions or properties. In one aspect, substitutions are conservative substitutions.

As used herein, a “conservative amino acid substitution” is substitution of an amino acid with an amino acid having a side chain or a similar chemical character. Similar amino acids for making conservative substitutions include those having an acidic side chain (glutamic acid, aspartic acid); a basic side chain (arginine, lysine, histidine); a polar amide side chain (glutamine, asparagine); a hydrophobic, aliphatic side chain (leucine, isoleucine, valine, alanine, glycine); an aromatic side chain (phenylalanine, tryptophan, tyrosine); a small side chain (glycine, alanine, serine, threonine, methionine); or an aliphatic hydroxyl side chain (serine, threonine). In addition, individual substitutions, deletions or additions that alter, add or delete, a single amino acid or a small percentage of amino acids in an encoded sequence can in some instances be categorized as “conservative substitutions.”

As used herein, “heterologous” or “exogenous” or “non-endogenous”, construct or sequence refers to a nucleic acid molecule or portion of a nucleic acid molecule that is not native to a host cell, but can be homologous to a nucleic acid molecule or portion of a nucleic acid molecule from the host cell. The source of the heterologous or exogenous nucleic acid molecule, construct or sequence can be from a different genus or species. In certain embodiments, a heterologous or exogenous nucleic acid molecule is added (for example, not endogenous or native) to cell or population of cells or genome or population of genomes by, for example, conjugation, transformation, transfection, transduction, electroporation, or the like, wherein the added molecule can integrate into the host genome or exist as extra-chromosomal genetic material (for example, as a plasmid or other form of self-replicating vector), and can be present in some instances in multiple copies. In addition, “heterologous” refers to a non-native protein or other activity encoded by an exogenous nucleic acid molecule introduced into the host cell, even if the host cell encodes a homologous protein or activity. In some embodiments, genetically modifying the population of tumor cells to express a tumor haplotype different from the tumor haplotype endogenous to the population of tumor cells includes modification that involves employing a “heterologous” or “exogenous” or “non-endogenous” sequence as part of the genetic modification.

As described herein, more than one heterologous or exogenous nucleic acid molecule can be introduced into a cell or population of cells as separate nucleic acid molecules, as a plurality of individually controlled genes, as a polycistronic nucleic acid molecule, as a single nucleic acid molecule encoding a fusion protein, or any combination thereof. For example, as disclosed herein, a host cell can be modified to express two or more heterologous or exogenous nucleic acid molecules encoding the desired genetic modification, for example, a tumor haplotype different from the tumor haplotype endogenous to the population of tumor cells. In some embodiments, the different haplotype allows for matching the tumor haplotype to a TCR specific for a minor histocompatibility (H) antigen peptide (for example, TCRα and TCRβ). When two or more exogenous nucleic acid molecules are introduced into a cell or population of cells, it is understood that the two or more exogenous nucleic acid molecules can be introduced as a single nucleic acid molecule (for example, on a single vector), on separate vectors, integrated into the host chromosome at a single site or multiple sites, or any combination thereof. The number of referenced heterologous nucleic acid molecules or protein activities refers to the number of encoding nucleic acid molecules or the number of protein activities, not the number of separate nucleic acid molecules introduced into a cell or population of cells.

As used herein, the term “endogenous” or “native” refers to a gene, protein, or activity that is normally present in a cell or population of cells. In some embodiments, a gene, protein or activity can be mutated, overexpressed, shuffled, duplicated or otherwise altered as compared to a parent gene or wild-type gene, protein or activity and could still considered to be endogenous or native to that particular cell or population of cells.

As used herein, the term “homologous” or “homolog” refers to a molecule or activity found in or derived from a host cell, species or strain. For example, a heterologous or exogenous nucleic acid molecule can be homologous to a native host cell gene, and can optionally have an altered expression level, a different sequence, an altered activity, or any combination thereof.

As used herein, the phrase “sequence identity” indicates the identity/similarity between two or more nucleic acid sequences, or two or more amino acid sequences, and is expressed in terms of the identity or similarity between the sequences. Sequence identity can be measured in terms of percentage identity; the higher the percentage, the more identical the sequences are. Sequence similarity can be measured in terms of percentage similarity (which takes into account conservative amino acid substitutions); the higher the percentage, the more similar the sequences are. Homologs or orthologs of nucleic acid or amino acid sequences possess a relatively high degree of sequence identity/similarity when aligned using standard methods. Methods of alignment of sequences for comparison are well known in the art. Various programs and alignment algorithms are described in: Smith & Waterman, Adv. Appl. Math. 2:482, 1981; Needleman & Wunsch, J. Mol. Biol. 48:443, 1970; Pearson & Lipman, Proc. Natl. Acad. Sci. USA 85:2444, 1988; Higgins & Sharp, Gene, 73:237-44, 1988; Higgins & Sharp, CABIOS 5:151-3, 1989; Corpet et al., Nuc. Acids Res. 16:10881-90, 1988; Huang et al. Computer Appls. in the Biosciences 8, 155-65, 1992; and Pearson et al., Meth. Mol. Bio. 24:307-31, 1994. Altschul et al., J. Mol. Biol. 215:403-10, 1990, presents a detailed consideration of sequence alignment methods and homology calculations. The NCBI Basic Local Alignment Search Tool (BLAST) (Altschul et al., J. Mol. Biol. 215:403-10, 1990) is available from several sources, including the National Center for Biological Information (NCBI, National Library of Medicine, Building 38A, Room 8N805, Bethesda, Md. 20894) and on the Internet, for use in connection with the sequence analysis programs blastp, blastn, blastx, tblastn and tblastx. Additional information can be found at the NCBI web site. BLASTN can be used to compare nucleic acid sequences, while BLASTP can be used to compare amino acid sequences. For comparisons of amino acid sequences of greater than about 30 amino acids, the Blast 2 sequences function is employed using the default BLOSUM62 matrix set to default parameters. Homologs are typically characterized by possession of at least 70% sequence identity counted over the full-length alignment with an amino acid sequence using the NCBI Basic Blast 2.0, gapped blastp with databases such as the nr or Swissprot database. Queries searched with the blastn program are filtered with DUST (Hancock and Armstrong, 1994, Comput. Appl. Biosci. 10:67-70). In addition, a manual alignment can be performed. Proteins with even greater similarity will show increasing percentage identities when assessed by this method, such as at least about 75%, 80%, 85%, 90%, 95%, 98%, or 99% sequence identity to a cargo protein or targeting moiety provided herein. When aligning short peptides (fewer than around 30 amino acids), the alignment is be performed using the Blast 2 sequences function, employing the PAM30 matrix set to default parameters (open gap 9, extension gap 1 penalties). Proteins with even greater similarity to the reference sequence will show increasing percentage identities when assessed by this method, such as at least about 60%, 70%, 75%, 80%, 85%, 90%, 95%, 98%, 99% sequence identity to a cargo moiety or targeting moiety provided herein. When less than the entire sequence is being compared for sequence identity, homologs will typically possess at least 75% sequence identity over short windows of 10-20 amino acids, and can possess sequence identities of at least 85%, 90%, 95% or 98% depending on their identity to the reference sequence. Methods for determining sequence identity over such short windows are described at the NCBI web site.

As used herein, the term “variable region” or “variable domain” refers to the domain of an immunoglobulin superfamily binding protein (for example, a TCR α-chain or β-chain (or γ chain and δ chain for γδ TCRs)) that is involved in binding of the immunoglobulin superfamily binding protein (for example, TCR) to antigen. The variable domains of the α-chain and β-chain (Vα and νβ, respectively) of a native TCR generally have similar structures, with each domain comprising four conserved framework regions (FRs) and three CDRs. The Vα domain is encoded by two separate DNA segments, the variable gene segment and the joining gene segment (V-J); the νβ domain is encoded by three separate DNA segments, the variable gene segment, the diversity gene segment, and the joining gene segment (V-D-J). A single Vα or νβ domain may be sufficient to confer antigen-binding specificity. Furthermore, TCRs that bind a particular antigen may be isolated using a Vα or νβ domain from a TCR that binds the antigen to screen a library of complementary Vα or νβ domains, respectively.

As used herein, the terms “complementarity determining region,” and “CDR,” are synonymous with “hypervariable region” or “HVR,” and are known in the art to refer to noncontiguous sequences of amino acids within TCR variable regions, which confer antigen specificity and/or binding affinity. In general, there are three CDRs in each α-chain variable region (αCDR1, αCDR2, αCDR3) and three CDRs in each β-chain variable region (βCDR1, βCDR2, βCDR3). While not being bound by theory, CDR3 is thought to be the main CDR responsible for recognizing processed antigen, while CDR1 and CDR2 mainly interact with the MHC, including MHC I.

### II. Haplotype Modification

The invention described here describes a method for delivering an HLA molecule to a mismatched tumor cell that expresses the appropriate TAA and rendering the tumor cell susceptible to killing by a T cell expressing the TAA targeted TCR.

Downregulation of HLA expression within tumor cells is a major mechanism that tumors utilize to escape immune surveillance (12, 13). Such down regulation of HLA by tumor cells leads to non-responsiveness in TCR-based immunotherapy. In fact, HLA class I loss or downregulation has been described in human tumors of different origin with percentages that range from 60% to 90%. The present invention addresses this need by providing methods reversing the HLA loss and/or downregulation.

In addition to engineering HLA expression to allow HLA-mismatched patients to become eligible to a certain TCR-T treatment, the methods described by the present invention also allow for engineering tumor cells to express a missing and/or different HLA, which can improve tumor killing efficacy in an autologous and/or allogenic settings in vivo.

The present invention provides methods for increasing the sensitivity of tumor cells to a TCR-engineered T cells (TCR-T) therapy comprising genetically modifying the tumor cells to express a haplotype, for example an HLA haplotype, different from the haplotype endogenous to the tumor cells. In some embodiments, the methods comprise methods for genetically modifying the tumor cells to express a desired HLA haplotype. In some embodiments, the methods comprise methods for genetically modifying the tumor cells to increase expression of a desired HLA haplotype. In some embodiments, the methods comprise methods for genetically modifying the tumor cells to increase expression of a desired HLA haplotype when the population of tumor cells are HLA negative.

In some embodiments, the methods comprise methods for increasing the sensitivity of a population of tumor cells to a TCR-engineered T cell (TCR-T) therapy, the method comprising genetically modifying the population of tumor cells to express a tumor haplotype different from the tumor haplotype endogenous to the population of tumor cells. In some embodiments, the increase in sensitivity is an increase in at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the population of tumor cells prior to genetically modifying the tumor haplotype. In some embodiments, the increase in sensitivity is an increase of at least 1-fold, at least 2-fold, at least 3-fold, at least 4-fold, at least 5-fold, at least 6-fold, at least 7-fold, at least 8-fold, at least 9-fold, or at least 10-fold, as compared to the population of tumor cells prior to genetically modifying the tumor haplotype. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments, the methods comprise methods of upregulating antigen presentation on the cellular surfaces of a population of tumor cells to increase expression of a tumor haplotype different from the tumor haplotype endogenous to the population of tumor cells. In some embodiments, the increase in expression is at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the population of tumor cells prior to genetically modifying the tumor haplotype. In some embodiments, the upregulating antigen presentation is an upregulation of at least 1-fold, at 2-fold, at least 3-fold, at least 4-fold, at least 5-fold, at least 6-fold, at least 7-fold, at least 8-fold, at least 9-fold, or at least 10-fold as compared to the population of tumor cells prior to genetically modifying the tumor haplotype. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments, the methods comprise methods of upregulating antigen presentation on the cellular surfaces of a population of tumor cells to increase sensitivity of the population of tumor cells to a TCR-engineered T cell (TCR-T) therapy comprising genetically modifying the population of tumor cells to express a tumor haplotype different from the tumor haplotype endogenous to the population of tumor cells. In some embodiments, the upregulating antigen presentation is an upregulation of at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the population of tumor cells prior to genetically modifying the tumor haplotype. In some embodiments, the upregulating antigen presentation is an upregulation of at least 1-fold, at least 2-fold, at least 3-fold, at least 4-fold, at least 5-fold, at least 6-fold, at least 7-fold, at least 8-fold, at least 9-fold, or at least 10-fold as compared to the population of tumor cells prior to genetically modifying the tumor haplotype. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments, the methods comprise methods of reversing downregulation of expression of a tumor haplotype gene in a population of tumor cells in order to increase sensitivity of the population of tumor cells to a TCR-engineered T cell (TCR-T) therapy, wherein the method comprises genetically modifying the population of tumor cells to express the tumor haplotype. In some embodiments, the reversing downregulation of expression of a tumor haplotype gene is a reversal of expression of at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the population of tumor cells prior to genetically modifying the tumor haplotype. In some embodiments, the reversing downregulation of expression of a tumor haplotype gene is a reversal of expression of at least 1-fold, at 2-fold, at least 3-fold, at least 4-fold, at least 5-fold, at least 6-fold, at least 7-fold, at least 8-fold, at least 9-fold, or at least 10-fold as compared to the population of tumor cells prior to genetically modifying the tumor haplotype. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments, the methods comprise methods for increasing HLA expression to render a population of tumor cells susceptible to autologous T cells, wherein the method comprises genetically modifying the population of tumor cells to express the HLA haplotype. In some embodiments, the increasing HLA expression to render a population of tumor cells susceptible to autologous T cells is an increase of at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the population of tumor cells genetically modifying the tumor haplotype. In some embodiments, the increasing HLA expression to render a population of tumor cells susceptible to autologous T cells is an increase o of at least 1-fold, at 2-fold, at least 3-fold, at least 4-fold, at least 5-fold, at least 6-fold, at least 7-fold, at least 8-fold, at least 9-fold, or at least 10-fold as compared to the population of tumor cells genetically modifying the tumor haplotype. In some embodiments, the methods comprise methods for genetically modifying the tumor cells to increase expression of a desired HLA haplotype when the population of tumor cells are HLA negative.

In some embodiments, the methods comprise methods for increasing HLA expression to render a population of tumor cells susceptible to allogeneic T cells, wherein the method comprises genetically modifying the population of tumor cells to express the HLA haplotype. In some embodiments, the increasing HLA expression to render a population of tumor cells susceptible to allogeneic T cells is an increase of at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the population of tumor cells prior to genetically modifying the tumor haplotype. In some embodiments, the increasing HLA expression to render a population of tumor cells susceptible to allogeneic T cells is an increase of at least 1-fold, at least 2-fold, at least 3-fold, at least 4-fold, at least 5-fold, at least 6-fold, at least 7-fold, at least 8-fold, at least 9-fold, or at least 10-fold as compared to the population of tumor cells prior to genetically modifying the tumor haplotype. In some embodiments, the methods comprise methods for genetically modifying the tumor cells to increase expression of a desired HLA haplotype when the population of tumor cells are HLA negative.

In some embodiments, the method further comprises expressing the tumor haplotype that is different from the tumor haplotype that is endogenous to the population of tumor cells in the genetically modified cell population.

In some embodiments, expressing the tumor haplotype that is different from the tumor haplotype that is endogenous to the population of tumor cells allows for targeting the population of tumor cells with the TCR-T.

In some embodiments, the methods comprise methods for increasing the sensitivity of a tumor cell to a TCR-engineered T cell (TCR-T) therapy comprising:
a) determining the tumor haplotype of the population of tumor cells;
b) contacting the population of tumor cells with a nucleic acid encoding a tumor haplotype different from the tumor haplotype endogenous to the tumor cells, wherein the tumor haplotype different from the tumor haplotype endogenous to the tumor cells is expressed, and wherein the population of tumor cells exhibit increased sensitivity to a TCR-T therapy.

In some embodiments, determining the tumor haplotype of the population of tumor cells can include any of a variety of methods for determining the haplotype, including PCR, sequencing, flow cytometry, as well as other methods for determining genetic profiles for a population of cells. In some embodiments, PCR and/or flow cytometry methods include commercially available methods and assays. In some embodiments, flow cytometry methods employ a Luminex platform (commercially available on the World Wide Web at luminexcorp.com/). In some embodiments, contacting can include transfection and/or transformation methods. In some embodiments, the tumor haplotype is an HLA haplotype. In some embodiments, determining the tumor haplotype can be employed using samples from tissue, including tumor tissue samples, as well as blood samples. In some embodiments, the sample is from a solid tumor, for example, a carcinoma, a sarcoma, and/or a lymphoma. In some embodiments, the sample is from a solid tumor as described herein in Section II, entitled “II. Solid Tumors for Treatment”.

In some embodiments, the tumor haplotype different from the tumor haplotype endogenous to the tumor cells is expressed and upregulates antigen presentation in the population of cells. In some embodiments, expression of the tumor haplotype different from the tumor results in an increase of at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to expression in the population of tumor cells prior to genetically modifying the tumor haplotype and upregulation of antigen presentation in the population of cells by at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the antigen presentation in the population of tumor cells prior to genetically modifying the tumor haplotype. In some embodiments, expression of the tumor haplotype different from the tumor results in an increase of at least 1-fold, at least 2-fold, at least 3-fold, at least 4-fold, at least 5-fold, at least 6-fold, at least 7-fold, at least 8-fold, at least 9-fold, or at least 10-fold as compared to expression in the population of tumor cells prior to genetically modifying the tumor haplotype and upregulation of antigen presentation in the population of cells of at least 1-fold, at least 2-fold, at least 3-fold, at least 4-fold, at least 5-fold, at least 6-fold, at least 7-fold, at least 8-fold, at least 9-fold, or at least 10-fold as compared to the antigen presentation in the population of tumor cells prior to genetically modifying the tumor haplotype. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments, the tumor haplotype different from the tumor haplotype endogenous to the tumor cells is expressed and reverses downregulation of expression of a tumor haplotype gene. In some embodiments, expression of the tumor haplotype different from the tumor results in an increase of at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to expression in the population of tumor cells prior to genetically modifying the tumor haplotype and reverses downregulation of expression in the population of cells by at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% as compared to the antigen presentation in the population of tumor cells prior to genetically modifying the tumor haplotype. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments, expression of the tumor haplotype different from the tumor results in an increase of at least 1-fold, at least 2-fold, at least 3-fold, at least 4-fold, at least 5-fold, at least 6-fold, at least 7-fold, at least 8-fold, at least 9-fold, or at least 10-fold as compared to expression in the population of tumor cells prior to genetically modifying the tumor haplotype and reverses downregulation of expression in the population of cells of at least 1-fold, at least 2-fold, at least 3-fold, at least 4-fold, at least 5-fold, at least 6-fold, at least 7-fold, at least 8-fold, at least 9-fold, or at least 10-fold as compared to the antigen presentation in the population of tumor cells prior to genetically modifying the tumor haplotype. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments, the methods provide a method for increasing HLA expression to render a population of tumor cells susceptible to a TCR-engineered T cell (TCR-T) therapy comprising:
a) determining the HLA haplotype of the population of tumor cells;
b) contacting the population of tumor cells with a nucleic acid encoding an HLA haplotype different from the HLA haplotype endogenous to the tumor cells, wherein the HLA haplotype different from the HLA haplotype endogenous to the tumor cells is expressed, and wherein the population of tumor cells exhibit increased sensitivity to a TCR-T therapy.

In some embodiments, determining the tumor haplotype of the population of tumor cells can include any of a variety of methods for determining the haplotype, sequence based assays, including PCR, or flow cytometry based assays (including FACS or other cell sorting based methods), exome sequencing, etc., as well as other methods for determining genetic profiles for a population of cells. In some embodiments, the tumor haplotype is an HLA haplotype.

In some embodiments, contacting can include infection, transfection and/or transformation methods involving the nucleic acids described herein. In some embodiments, contacting can include methods involving viral infection, based on the particular viral vector employed.

In some embodiments of the methods described herein, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells are capable of expressing the tumor haplotype that is different from the tumor haplotype endogenous to the population of tumor cells. In some embodiments of the methods described herein, at least 1-fold, at least 2-fold, at least 3-fold, at least 4-fold, at least 5-fold, at least 6-fold, at least 7-fold, at least 8-fold, at least 9-fold, or at least 10-fold or more of the population of tumor cells are capable of expressing the tumor haplotype that is different from the tumor haplotype endogenous to the population of tumor cells. In some embodiments, the tumor haplotype is an HLA haplotype. In some embodiments, the methods comprise methods for genetically modifying the tumor cells to increase expression of a desired HLA haplotype when the population of tumor cells are HLA negative.

In some embodiments of the methods described herein, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells express the tumor haplotype that is different from the tumor haplotype endogenous to the population of tumor cells. In some embodiments of the methods described herein, at least 1-fold, at least 2-fold, at least 3-fold, at least 4-fold, at least 5-fold, at least 6-fold, at least 7-fold, at least 8-fold, at least 9-fold, or at least 10-fold or more of the population of tumor cells express the tumor haplotype that is different from the tumor haplotype endogenous to the population of tumor cells. In some embodiments, the tumor haplotype is an HLA haplotype. In some embodiments, the methods comprise methods for genetically modifying the tumor cells to express a desired HLA haplotype when the population of tumor cells are HLA negative.

In some embodiments of the methods described herein, at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells stably express the tumor haplotype that is different from the tumor haplotype endogenous to the population of tumor cells. In some embodiments of the methods described herein, at least 1-fold, at least 2-fold, at least 3-fold, at least 4-fold, at least 5-fold, at least 6-fold, at least 7-fold, at least 8-fold, at least 9-fold, or at least 10-fold or more of the population of tumor cells stably express the tumor haplotype that is different from the tumor haplotype endogenous to the population of tumor cells. In some embodiments, the tumor haplotype is an HLA haplotype. In some embodiments, the methods comprise methods for genetically modifying the tumor cells to stably express desired HLA haplotype when the population of tumor cells are HLA negative.

### I. Nucleic Acids, Viral and Non-Viral Vectors

In some embodiments, the present invention employs a nucleic acid in the methods genetically modifying the population of tumor cells. In some embodiments, the vector is a viral vector. In some embodiments, the vector is a non-viral vector.

In some embodiments, the construct for genetically modifying the population of tumor cells and producing a polypeptide of interest can be accomplished by using any suitable molecular biology engineering technique known in the art. To obtain efficient transcription and translation, a polynucleotide in each transgene construct of the present disclosure includes, in certain embodiments, at least one appropriate expression control sequence (also called a regulatory sequence), such as a leader sequence and particularly a promoter operably linked to the nucleotide sequence encoding the polypeptide of interest.

In some embodiments, the construct for genetically modifying the population of tumor cells encodes a selection marker. In some embodiments, the construct for genetically modifying the population of tumor cells encodes a selection marker comprises: CD34, truncated CD34, and/or LNGF-R (also known as low-affinity nerve growth factor receptor). In some embodiments, the construct for genetically modifying the population of tumor cells encodes a selection marker that results in the tumor cells expressing CD34, truncated CD34, and/or LNGF-R. In some embodiments, the construct for genetically modifying the population of tumor cells encodes a selection marker that results in the tumor cells expressing CD34. In some embodiments, the construct for genetically modifying the population of tumor cells encodes a selection marker that results in the tumor cells expressing truncated CD34. In some embodiments, the construct for genetically modifying the population of tumor cells encodes a selection marker that results in the tumor cells expressing LNGF-R.

In some embodiments, the present invention employs viral and/or non-viral vectors in the methods described herein. In some embodiments, the vector is a viral vector. In some embodiments, the vector is a non-viral vector. In some embodiments, the vector comprises a nucleic acid as described herein.

In some embodiments, the present invention employs the use of a vector in a method for increasing the sensitivity of a population of tumor cells to a TCR-engineered T cell (TCR-T) therapy. In some embodiments, the present invention employs the use of a vector in a method for increasing the sensitivity of a population of tumor cells to a TCR-engineered T cell (TCR-T) therapy comprising genetically modifying the population of tumor cells to express a tumor haplotype different from the tumor haplotype endogenous to the tumor cells.

In some embodiments, the present invention employs the use of a vector in a method of upregulating antigen presentation on the cellular surfaces of a population of tumor cells to increase sensitivity of the population of tumor cells to a TCR-engineered T cell (TCR-T) therapy comprising genetically modifying the population of tumor cells to express a tumor haplotype different from the tumor haplotype endogenous to the tumor cells.

In some embodiments, the present invention employs the use of a vector in a method of reversing downregulation of expression of a tumor haplotype gene in a population of tumor cells in order to increase sensitivity of the population of tumor cells to a TCR-engineered T cell (TCR-T) therapy, wherein the method comprises genetically modifying the population of tumor cells to express the tumor haplotype.

In some embodiments, the present invention employs the use of a vector in a method for increasing HLA expression to render a population of tumor cells susceptible to allogeneic T cells, wherein the method comprises genetically modifying the population of tumor cells to express the HLA haplotype. In some embodiments, the methods comprise methods for genetically modifying the tumor cells to increase HLA expression when the population of tumor cells are HLA negative.

In some embodiments, the present invention employs the use of a vector in a method for increasing HLA expression to render a population of tumor cells susceptible to autologous T cells, wherein the method comprises genetically modifying the population of tumor cells to express the HLA haplotype. In some embodiments, the methods comprise methods for genetically modifying the tumor cells to increase HLA expression when the population of tumor cells are HLA negative.

In some embodiments, the method comprises contacting the population of tumor cell with a nucleic acid encoding the tumor haplotype that is different from the tumor haplotype endogenous to the population of tumor cells. In some embodiments, the nucleic acid is contained within a vector. In some embodiments, the nucleic acid is contained within a non-viral vector. In some embodiments, the nucleic acid is contained within a viral vector. In some embodiments, the nucleic acid expresses the tumor haplotype that is different from the tumor haplotype endogenous to the population of tumor cells. In some embodiments, the nucleic acid encodes for the polypeptide that induces the tumor haplotype that is different from the tumor haplotype endogenous to the population of tumor cells.

In some embodiments, the method comprises contacting the population of tumor cells with a vector encoding the tumor haplotype that is different from the tumor haplotype endogenous to the population of tumor cells. In some embodiments, the vector expresses the tumor haplotype that is different from the tumor haplotype endogenous to the population of tumor cells. In some embodiments, the vector comprises the nucleic acid that expresses the tumor haplotype that is different from the tumor haplotype endogenous to the population of tumor cells. In some embodiments, the vector expresses the polypeptide that induces the tumor haplotype that is different from the tumor haplotype endogenous to the population of tumor cells.

In some embodiments, the nucleic acid or vector is transfected into the population of tumor cells such that there is stable expression of the tumor haplotype that is different from the tumor haplotype endogenous to the population of tumor cells.

In some embodiments, the nucleic acid or vector is transformed into the population of tumor cells such that there is stable expression of the tumor haplotype that is different from the tumor haplotype endogenous to the population of tumor cells.

In some embodiments, the nucleic acid or vector is inserted into the population of tumor cells such that there is stable expression of the tumor haplotype that is different from the tumor haplotype endogenous to the population of tumor cells.

In some embodiments, the nucleic acid or vector is integrated into the population of tumor cells such that there is stable expression of the tumor haplotype that is different from the tumor haplotype endogenous to the population of tumor cells.

In some embodiments, the nucleic acid or vector is stably integrated into the genome of the population of tumor cells. In some embodiments, the vector is stably integrated into the genome of the population of tumor cells. In some embodiments, the nucleic acid is stably integrated into the genome of the population of tumor cells.

In some embodiments, the nucleic acid or vector is inserted into at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells such that there is expression of the tumor haplotype encoded by the nucleic acid or vector. In some embodiments, the tumor haplotype is an HLA haplotype. In some embodiments, the tumor haplotype is the absence of an HLA haplotype.

In some embodiments, the vector is transfected into at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells such that there is expression of the tumor haplotype encoded by the nucleic acid or vector. In some embodiments, the vector is transfected into at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells such that there is stable expression of the tumor haplotype encoded by the nucleic acid or vector. In some embodiments, the tumor haplotype is an HLA haplotype. In some embodiments, the tumor haplotype is the absence of an HLA haplotype.

In some embodiments, the nucleic acid or vector is integrated into at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99% or more of the population of tumor cells such that there is stable expression of the tumor haplotype encoded by the nucleic acid or vector. In some embodiments, the tumor haplotype is an HLA haplotype. In some embodiments, the tumor haplotype is the absence of an HLA haplotype.

In some embodiments, stable expression of the tumor haplotype in the population of tumor cells is indicated by expression of the tumor haplotype that is different from the tumor haplotype endogenous to the population of tumor cells for at least 12 hours, at least 24 hours, at least 1 week, at least 2 weeks, at least 3 weeks, at least 4 weeks, or at least 1 month. In some embodiments, the tumor haplotype is an HLA haplotype. In some embodiments, the tumor haplotype is the absence of an HLA haplotype.

a. Viral Vectors

Multiple mechanisms of tumor killing in combinational therapy of oncolytic virus and TCR-T based therapy. In fact, the first oncolytic virus therapy approved by the FDA is T-VEC, an oncolytic immunotherapy (OI) derived from herpes simplex virus type-1 designed to selectively replicate within tumors and to produce GM-CSF to enhance systemic antitumor immune responses. In some embodiments, the invention described herein proposes using an oncolytic virus encoding an HLA molecule instead of GM-CSF, to specifically switch the HLA type in the tumor cells to render them sensitive to an available TCR. The oncolytic virus encoding GM-CSF leads to tumor destruction 2-3 days after intra-tumoral injection, attracting dendritic cells and macrophages to the tumor site and inducing tumor-reactive T cell responses in vivo. In addition to oncolysis driven tumor destruction, oncolytic delivery of an HLA molecule may lead to tumor cells susceptible to TCR-T mediated cytotoxicity which may occur weeks after adoptive transfer of TCR-T in vivo. Tumor cells that have acquired a mismatched or allogeneic HLA but lack tumor antigen expression may escape TCR-T mediated killings. However, these tumor cells expressing mismatched or allogeneic HLA but having antigen loss may effectively become targets of host versus tumor effects in longer terms due to expression of allogeneic HLA.

In some embodiments, an oncolytic virus can be employed as a vehicle to deliver allogeneic HLA molecules to tumor cells. To overcome the downregulation of HLA expression in tumor cells and to gain expression of a mismatched HLA, oncolytic viruses encoding selected HLA molecules can be used to deliver the molecules to tumor cells. Viruses suitable for HLA gene delivery include vaccinia (pox), adenovirus, herpes simplex virus (HSV), coxsackie virus, poliovirus, measles virus and Newcastle disease virus. Tumor selective expression can be achieved with each of these viruses through deletion of genes specifically required for virus replication in normal cells but not required for replication in tumor cells. For example, deletion of the viral thymidine kinase genes in vaccinia virus has little effect on viral replication in tumors that typically have a large pool of nucleotides but abolishes replication in normal cells that express low levels of thymidine kinase. Additionally, anti-viral responses in tumor cells are frequently dysfunctional. In healthy tissues, interferons and interferon related factors limit viral replication and boosts viral clearance, while limited interferon responses in tumors permit viral replication. Tumor selective expression can also be achieved by placing viral genes under control of tumor specific promoters, such as the telomerase reverse transcriptase (TERT) promoter. Similarly, use of tissue specific promoters, for example, the promoter of the gene encoding prostate specific antigen can be used to restrict expression to the prostate, which is a non-vital organ. To date, there is one oncolytic virus—a genetically modified form of a herpes virus for treating melanoma—that has been approved by the Food and Drug Administration (FDA), though a number of viruses are being evaluated as potential treatments for cancer are in clinical trials. According to the present invention, any of a number of oncolytic viruses can be employed with the described methods.

Oncolytic viruses can be delivered intratumorally and/or intravenously. Both modes of delivery have been shown to be effective in animal models. In the clinic, guided intratumoral injection has been used most extensively, and indeed is the only option for certain viruses such as HSV. However, intravenous delivery of vaccinia and adenovirus has been demonstrated clinically. Intratumoral injection has the disadvantage of being applicable only to accessible tumors or metastases, such as tumors in the liver.

Recently, it has been reported that oncolytic viruses have the ability to reverse the apparent down regulation of HLA expression in tumors and convert “cold” tumors into “hot” inflamed tumors. The use of oncolytic viruses expressing either a patient's own HLA haplotype or a mis-matched haplotype might then have a dual benefit in modulating the immunogenicity of the tumor micro-environment.

In some embodiments, viral vector is selected from the group consisting of a vaccinia (pox) virus vector, herpes simplex virus vector, myxoma virus, coxsackie virus vector, poliovirus vector, Newcastle disease virus vector, retrovirus vector (including lentivirus vector or a pseudotyped vector), an adenovirus vector, an adeno-associated virus vector, a simian virus vector, a sendai virus vector, measles virus vector, foam virus vector, alphavirus vector, and vesicular stomatitis virus vector. In some embodiments, the viral vector is selected from the group consisting of a vaccinia (pox) virus vector, herpes simplex virus vector, and myxoma virus. In some embodiments, the viral vectors are vaccinia based viral vectors, herpes simplex viral based vectors, HSV viral based vectors, and myxoma viral based vectors.

### B. Vaccinia

The present invention can further employ one of a number of vaccinia viruses as the vector employed for inducing the haplotype modification.

### 1.Vaccinia Vector Exemplary Embodiment

In some embodiments, the haplotype modification is facilitated by employing a vaccinia based viral technology, for example, and including the vaccinia platform, as described in International Patent Publication No. WO 2019/134048, incorporated herein by reference in its entirety. In some embodiments, the viral vector is a vaccinia viral vector. In some embodiments, the viral vector is a vaccinia viral vector comprising haplotype modifying sequences. In some embodiments, when viral vector is a vaccinia (pox) virus vector, the administration route is systemic.

In some embodiments, the present invention makes the use of orthopoxviruses for the treatment of cancer. In particular, the present invention can make sure of the enhanced oncolytic activity, spread of infection, and safety results engendered when a orthopoxvirus is genetically modified to contain deletions in one or more, or all, of the following genes: C2L, C1L, NIL, N2L, MIL, M2L, K1L, K2L, K3L, K4L, K5L, K6L, K7R, F1L, F2L, F3L, B14R, B15R, B16R, B17L, B18R, B19R, B20R, K ORF A, K ORF B, B ORF E, B ORF F, B ORF G, B21R, B22R, B23R, B24R, B25R, B26R, B27R, B28R, and B29R. Genetically modified orthopoxviruses, such as vaccinia viruses (e.g., Copenhagen, Western Reserve, Wyeth, Lister, EM63, ACAM2000, LC16m8, CV-1, modified vaccinia Ankara (MV A), Dairen I, GLV-1h68, IE1D-J, L-IVP, LC16m8, LC16mO, Tashkent, Tian Tan, and WAU86/88-1 viruses) that exhibit mutations in one or more, or all, of these genes may exhibit an array of beneficial features, such as improved oncolytic ability, replication in tumors, infectivity, immune evasion, tumor persistence, capacity for incorporation of exogenous DNA sequences, and/or amenability for large scale manufacturing. The present invention further contemplates the use of orthopox viruses further genetically modified to contain deletions in the B8R gene. In some embodiments, the vector may or may not include a deletion of the B8R gene.

In some embodiments, the nucleic acid that includes a recombinant orthopoxvirus genome, wherein the recombinant orthopoxvirus genome has a deletion of at least 2, 3, 4, 5, 6, 7, 8, 9, 10, 11, 12, 13, 14, 15, 16, 17, 18, 19, 20, 21 or 22 genes, each independently selected from the group consisting of C2L, C1L, NIL, N2L, MIL, M2L, K1L, K2L, K3L, K4L, K5L, K6L, K7R, F1L, F2L, F3L, B14R, B15R, B16R, B17L, B18R, B19R, B20R. In some embodiments, the deletion includes each of C2L, C1L, NIL, N2L, MIL, M2L, K1L, K2L, K3L, K4L, K5L, K6L, K7R, F1L, F2L, F3L, B14R, B15R, B16R, B17L, B18R, B19R, B20R genes. In some embodiments, the recombinant orthopoxvirus genome may further include a deletion of the B8R gene.

In some embodiments, the nucleic acid includes a recombinant orthopoxvirus genome, wherein the recombinant orthopoxvirus genome has a deletion of at least 1 gene selected from the group consisting of B14R, B16R, B17L, B18R, B19R, and B20R. In some embodiments, the deletion includes at least 2, 3, 4, or 5 genes, each independently selected from the group consisting of B14R, B16R, B17L, B18R, B19R, and B20R. In some embodiments, the deletion includes each of B14R, B16R, B17L, B18R, B19R, and B20R. In some embodiments, the recombinant orthopoxvirus genome may further include a B8R deletion.

In some embodiments, the nucleic acid includes a recombinant orthopoxvirus genome, wherein the recombinant orthopoxvirus genome has a deletion of at least 1 gene selected from the group consisting of C2L, C1L, NIL, N2L, MIL, M2L, K1L, K2L, K3L, K4L, K5L, K6L, K7R, F1L, F2L, and F3L. In some embodiments, the deletion includes at least 2, 3, 4, 5, 6, 7, 8, 9, 10, 11, 12, 13, 14, or 15 genes, each independently selected from the group consisting of C2L, C1L, NIL, N2L, MIL, M2L, K1L, K2L, K3L, K4L, K5L, K6L, K7R, F1L, F2L, and F3L. In some embodiments, the deletion includes each of C2L, C1L, NIL, N2L, MIL, M2L, K1L, K2L, K3L, K4L, K5L, K6L, K7R, F1L, F2L, and F3L. In some embodiments, the recombinant orthopoxvirus genome may further include a B8R deletion.

In some embodiments, the recombinant orthopoxvirus genome has a deletion of at least 1 gene selected from the group of inverted terminal repeat (ITR) genes consisting of B21R, B22R, B23R, B24R, B25R, B26R, B27R, B28R, and B29R. In some embodiments, the deletion includes at least 2, 3, 4, 5, 6, 7, or 8 genes, each independently selected from the group of ITR genes consisting of B21R, B22R, B23R, B24R, B25R, B26R, B27R, B28R, and B29R. In some embodiments, the deletion includes each of B21R, B22R, B23R, B24R, B25R, B26R, B27R, B28R, and B29R. In some embodiments, disclosed herein, the recombinant orthopoxvirus genome may further include a B8R deletion.

In some embodiments, the vaccinia virus is a strain selected from the group consisting of Copenhagen, Western Reserve, Wyeth, Lister, EM63, ACAM2000, LC16m8, CV-1, modified vaccinia Ankara (MV A), Dairen I, GLV-lh68, IHD-J, L-IVP, LC16m8, LC16mO, Tashkent, Tian Tan, and WAU86/88-1. In some embodiments, the vaccinia virus is a strain selected from the group consisting of Copenhagen, Western Reserve, Tian Tan, Wyeth, and Lister. In some embodiments, the vaccinia virus is a Copenhagen strain vaccinia virus. In some embodiments, the vaccinia virus is a Western Reserve vaccinia virus.

In some embodiments, one or more, or all, of the deletions is a deletion of the entire polynucleotide encoding the corresponding gene. In some embodiments, one or more, or all, of the deletions is a deletion of a portion of the polynucleotide encoding the corresponding gene, such that the deletion is sufficient to render the gene nonfunctional, e.g., upon introduction into a host cell.

### 2.Vaccinia Vector Exemplary Embodiment

In some embodiments, the haplotype modification is facilitated by employing a vaccinia based viral technology, for example, and including the vaccinia platform, as described in International Patent Publication No. WO 2019/089755A1, incorporated herein by reference in its entirety. In some embodiments, the viral vector is a vaccinia viral vector. In some embodiments, the viral vector is a vaccinia viral vector comprising haplotype modifying sequences. In some embodiments, when viral vector is a vaccinia (pox) virus vector, the administration route is systemic.

In some embodiments, the vaccinia viral vector can comprise the modification in the genome of the virus. In some embodiments, the vaccinia viral vector is capable of enhanced production of enveloped extracellular form (EEV) of the virus. In some embodiments, the vaccinia viral vector can comprise a mutation or a deletion of the B5R gene, wherein said deletion is a partial deletion. In some embodiments, the vaccinia viral vector can comprise a mutation or a deletion in a SCR region of the B5R gene, wherein said SCR region comprises SCR1, SCR3, SCR4, or any combinations thereof, and wherein the SCR region does not comprise SCR2.

In some embodiments, the vaccinia viral vector can comprise mutation or deletion of the B5R gene. In some embodiments, the deletion can be a partial deletion of the B5R gene.

In some embodiments, the vaccinia viral vector can comprise a modification in the genome of the virus, wherein the modification can comprise a mutation or a deletion of the A52R gene. In some embodiments, the vaccinia viral vector can comprise the deletion of the A52R gene.

In some embodiments, the vaccinia viral vector can further comprise at least one additional modification in the genome of the virus, wherein the additional modification can comprise a mutation or a deletion of a further viral gene.

In some embodiments, the further viral gene can comprise at least one of F13L, A36R, A34R, A33R, B8R, B18R, SPI-1, SPI-2, B15R, VGF, E3L, K3L, A41L, K7R, and NIL, and a functional domain or fragment or variant thereof, or any combinations thereof.

In some embodiments, the vaccinia viral vector can further comprise at least one additional exogenous nucleic acid, including for example. In some embodiments, the at least one additional exogenous nucleic acid can comprise a nucleic acid coding for LIGHT (Lymphotoxins-like, exhibits Inducible expression, and competes with HSV Glycoprotein D for Herpesvirus entry mediator (HVEM), a receptor expressed by T lymphocytes) sequence. In some embodiments, the vaccinia viral vector can further comprise an exogenous nucleic acid that codes for a viral VH1 protein. In some embodiments, the modified vaccinia viral vector can comprise the exogenous nucleic acid coding for the viral VH1 protein, wherein the exogenous nucleic acid can be from a genome of a poxvirus, wherein the poxvirus is not a vaccinia virus. In some embodiments, the poxvirus can comprise a measles virus, a poliovirus, a poxvirus, a vaccinia virus, an adenovirus, an adeno associated virus, a herpes simplex virus, a vesicular stomatitis virus, a reovirus, a Newcastle disease virus, a senecavirus, a lentivirus, a mengovirus, and/or a myxomavir.

In some embodiments, the vaccinia viral vector genome can comprise a thymidine kinase gene. In some embodiments, a thymidine kinase gene can be deleted from the viral genome. In some embodiments, the vaccinia viral vector can further comprise a thymidine kinase gene from a herpes simplex virus.

### 3.Vaccinia Vector Exemplary Embodiment

In some embodiments, the haplotype modification is facilitated by employing a vaccinia based viral technology, for example, and including the vaccinia platform, as described in United States Patent Publication No. US 2020/0215132, incorporated herein by reference in its entirety. In some embodiments, the viral vector is a vaccinia viral vector. In some embodiments, the viral vector is a vaccinia viral vector comprising haplotype modifying sequences. In some embodiments, when viral vector is a vaccinia (pox) virus vector, the administration route is systemic.

In some embodiments, the vaccinia vector employed in the haplotype modification is a chimeric poxvirus comprises a nucleotide sequence having a sequence identity of at least 70% (80%, 85%, 90%, 95%, 98%) to SEQ ID NO:45 or SEQ ID NO:46 (SEQ ID NO:1 and SEQ ID NO:2 from US 2020/0215132; provide herein) or having a having a sequence identity of at least 70% (80%, 85%, 90%, 95%, 98%) to SEQ ID NO:1 or SEQ ID NO:2 (both from US 2020/0215132) that has been modified by deletion of the TK gene). The recombinant poxvirus is oncolytic and can infect and kill certain cancer cells.

In some embodiments, the nucleotide sequence having a sequence identity of at least 70% (80%, 85%, 90%, 95%, or 98%) to SEQ ID NO:45 or SEQ ID NO:46, includes: (i) nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS; (ii) one or more haplotype modifying nucleic acid sequences; or (iii) a detectable moiety-encoding nucleic acid sequence.

In another aspect the nucleotide sequence having a sequence identity of at least 70% (80%, 85%, 90%, 95%, or 98%) to SEQ ID NO:45, includes: (i) nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, and vaccinia virus strain AS; (ii) one or more haplotype modifying nucleic acid sequences; or (iii) a detectable moiety-encoding nucleic acid sequence.

In another aspect the nucleotide sequence having a sequence identity of at least 70% to SEQ ID NO:46, includes: (i) nucleic acid fragments from orf virus strain NZ2 and pseudocowpox virus strain TJS; (ii) one or more haplotype modifying nucleic acid sequences; or (iii) a detectable moiety-encoding nucleic acid sequence.

In an aspect the nucleotide sequence having a sequence identity of at least 70% (80%, 85%, 90%, 95%, or 98%) to SEQ ID NO:45 or SEQ ID NO:46, includes: (i) nucleic acid fragments from at least two poxvirus strains selected from the group consisting of cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, vaccinia virus strain AS, orf virus strain NZ2 and pseudocowpox virus strain TJS; (ii) one or more haplotype modifying nucleic acid sequences; (iii) one or more nucleic acid binding sequences; or (iv) a detectable moiety-encoding nucleic acid sequence.

In another aspect the nucleotide sequence having a sequence identity of at least 70% (80%, 85%, 90%, 95%, or 98%) to SEQ ID NO:45, includes: (i) nucleic acid fragments from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic, and vaccinia virus strain AS; (ii) one or more haplotype modifying nucleic acid sequences; (iii) one or more nucleic acid binding sequences; or (iv) a detectable moiety-encoding nucleic acid sequence.

In another aspect the nucleotide sequence having a sequence identity of at least 70% (80%, 85%, 90%, 95%, or 98%) (80%, 85%, 90%, 95%, or 98%) to SEQ ID NO:46, includes: (i) nucleic acid fragments from orf virus strain NZ2 and pseudocowpox virus strain TJS; (ii) one or more anti-cancer nucleic acid sequences; (iii) one or more haplotype modifying nucleic acid sequences; or (iv) a detectable moiety-encoding nucleic acid sequence.

In embodiments, the nucleic acid fragments are from cowpox virus strain Brighton, raccoonpox virus strain Herman, rabbitpox virus strain Utrecht, vaccinia virus strain WR, vaccinia virus strain IHD, vaccinia virus strain Elstree, vaccinia virus strain CL, vaccinia virus strain Lederle-Chorioallantoic and vaccinia virus strain AS.

In some embodiments, the nucleic acid sequence includes nucleic acid fragments from cowpox virus strain Brighton and raccoonpox virus strain Herman. In embodiments, the nucleic acid sequence includes nucleic acid fragments from cowpox virus strain Brighton and rabbitpox virus strain Utrecht. In embodiments, the nucleic acid sequence includes nucleic acid fragments from cowpox virus strain Brighton and vaccinia virus strain WR. In embodiments, the nucleic acid sequence includes nucleic acid fragments from cowpox virus strain Brighton and vaccinia virus strain IHD. In embodiments, the nucleic acid sequence includes nucleic acid fragments from cowpox virus strain Brighton and vaccinia virus strain Elstree. In embodiments, the nucleic acid sequence includes nucleic acid fragments from cowpox virus strain Brighton and vaccinia virus strain CL. In embodiments, the nucleic acid sequence includes nucleic acid fragments from cowpox virus strain Brighton and vaccinia virus strain Lederle-Chorioallantoic. In embodiments, the nucleic acid sequence includes nucleic acid fragments from cowpox virus strain Brighton and vaccinia virus strain AS. In embodiments, the nucleic acid sequence includes nucleic acid fragments from cowpox virus strain Brighton and orf virus strain NZ2. In embodiments, the nucleic acid sequence includes nucleic acid fragments from cowpox virus strain Brighton and pseudocowpox virus strain TJS.

In some embodiments, the nucleic acid sequence includes nucleic acid fragments from rabbitpox virus strain Utrecht and vaccinia virus strain WR. In embodiments, the nucleic acid sequence includes nucleic acid fragments from rabbitpox virus strain Utrecht and vaccinia virus strain IHD. In embodiments, the nucleic acid sequence includes nucleic acid fragments from rabbitpox virus strain Utrecht and vaccinia virus strain Elstree. In embodiments, the nucleic acid sequence includes nucleic acid fragments from rabbitpox virus strain Utrecht and vaccinia virus strain CL. In embodiments, the nucleic acid sequence includes nucleic acid fragments from rabbitpox virus strain Utrecht and vaccinia virus strain Lederle-Chorioallantoic. In embodiments, the nucleic acid sequence includes nucleic acid fragments from rabbitpox virus strain Utrecht and vaccinia virus strain AS. In embodiments, the nucleic acid sequence includes nucleic acid fragments from rabbitpox virus strain Utrecht and orf virus strain NZ2. In embodiments, the nucleic acid sequence includes nucleic acid fragments from rabbitpox virus strain Utrecht and pseudocowpox virus strain TJS.

In embodiments, the nucleic acid sequence includes nucleic acid fragments from vaccinia virus strain WR and vaccinia virus strain IHD. In embodiments, the nucleic acid sequence includes nucleic acid fragments from vaccinia virus strain WR and vaccinia virus strain Elstree. In embodiments, the nucleic acid sequence includes nucleic acid fragments from vaccinia virus strain WR and vaccinia virus strain CL. In embodiments, the nucleic acid sequence includes nucleic acid fragments from vaccinia virus strain WR and vaccinia virus strain Lederle-Chorioallantoic. In embodiments, the nucleic acid sequence includes nucleic acid fragments from vaccinia virus strain WR and vaccinia virus strain AS. In embodiments, the nucleic acid sequence includes nucleic acid fragments from vaccinia virus strain WR and orf virus strain NZ2. In embodiments, the nucleic acid sequence includes nucleic acid fragments from vaccinia virus strain WR and pseudocowpox virus strain TJS.

In embodiments, the nucleic acid sequence includes nucleic acid fragments from vaccinia virus strain IHD and vaccinia virus strain Elstree. In embodiments, the nucleic acid sequence includes nucleic acid fragments from vaccinia virus strain IHD and vaccinia virus strain CL. In embodiments, the nucleic acid sequence includes nucleic acid fragments from vaccinia virus strain IHD and vaccinia virus strain Lederle-Chorioallantoic. In embodiments, the nucleic acid sequence includes nucleic acid fragments from vaccinia virus strain IHD and vaccinia virus strain AS. In embodiments, the nucleic acid sequence includes nucleic acid fragments from vaccinia virus strain IHD and orf virus strain NZ2. In embodiments, the nucleic acid sequence includes nucleic acid fragments from vaccinia virus strain IHD and pseudocowpox virus strain TJS.

In embodiments, the nucleic acid sequence includes nucleic acid fragments from vaccinia virus strain Elstree and vaccinia virus strain CL. In embodiments, the nucleic acid sequence includes nucleic acid fragments from vaccinia virus strain Elstree and vaccinia virus strain Lederle-Chorioallantoic. In embodiments, the nucleic acid sequence includes nucleic acid fragments from vaccinia virus strain Elstree and vaccinia virus strain AS. In embodiments, the nucleic acid sequence includes nucleic acid fragments from vaccinia virus strain Elstree and orf virus strain NZ2. In embodiments, the nucleic acid sequence includes nucleic acid fragments from vaccinia virus strain Elstree and pseudocowpox virus strain TJS.

In embodiments, the nucleic acid sequence includes nucleic acid fragments from vaccinia virus strain CL and vaccinia virus strain Lederle-Chorioallantoic. In embodiments, the nucleic acid sequence includes nucleic acid fragments from vaccinia virus strain CL and vaccinia virus strain AS. In embodiments, the nucleic acid sequence includes nucleic acid fragments from vaccinia virus strain CL and orf virus strain NZ2. In embodiments, the nucleic acid sequence includes nucleic acid fragments from vaccinia virus strain CL and pseudocowpox virus strain TJS.

In embodiments, the nucleic acid sequence includes nucleic acid fragments from vaccinia virus strain Lederle-Chorioallantoic and vaccinia virus strain AS. In embodiments, the nucleic acid sequence includes nucleic acid fragments from vaccinia virus strain Lederle-Chorioallantoic and orf virus strain NZ2. In embodiments, the nucleic acid sequence includes nucleic acid fragments from vaccinia virus strain Lederle-Chorioallantoic and pseudocowpox virus strain TJS.

In embodiments, the nucleic acid sequence includes nucleic acid fragments from vaccinia virus strain AS and orf virus strain NZ2. In embodiments, the nucleic acid sequence includes nucleic acid fragments from vaccinia virus strain AS and pseudocowpox virus strain TJS. In embodiments, the nucleic acid sequence includes nucleic acid fragments from orf virus strain NZ2 and pseudocowpox virus strain TJS.

c. Herpes Simplex Virus (HSV)

### 1. HSV Vector Exemplary Embodiment

In some embodiments, the haplotype modification is facilitated by employing a herpes simplex virus based viral technology, for example, and including the herpes simplex virus platform, as described in International Patent Publication No. WO 2017/132552, incorporated herein by reference in its entirety. In some embodiments, the viral vector is a herpes simplex virus vector. In some embodiments, the viral vector is a herpes simplex virus vector comprising haplotype modifying sequences. In some embodiments, when the viral vector is a herpes simplex virus vector, the administration route is intratumoral.

In some embodiments, the present invention provides for a recombinant oncolytic virus comprising one or more copies of one or more target sequences can be inserted into a locus of one or more viral genes required for viral replication. In some embodiments, the virus is a herpes simplex virus, an adenovirus, a polio virus, a vaccinia virus, a measles virus, a vesicular stomatitis virus, an orthomyxovirus, a parvovirus, a maraba virus or a coxsackievirus. In some embodiments, the virus is a herpes simplex virus and wherein the one or more viral genes required for viral replication is selected from the group consisting of UL1, UL5, UL6, UL7, UL8, UL9, UL11, UL12, UL14, UL15. UL17, 1X18, UL19. UL20, UL22, 1X25, 1X26, UL26.5, UL27, UL28, UL29, UL30, UL31, UL32, UL33, UL34, UL35, UL36, UL37, UL38, UL39, UL40, UL42, UL48, UL49, UL52, UL53, UL54, ICPO, ICP4, ICP22, ICP27, ICP47, gamma-34.5, US 3, US4, US5, US6, US7, US8, US9, US10, US 11, and US12. In some embodiments, the haplotype modifying sequence can be incorporated into the 5′ untranslated region (UTR) or 3′ UTR of the one or more viral genes required for viral replication. In some embodiments, the haplotype modifying sequences are inserted into the ICP4, ICP27, UL19, and/or UL30 locus.

### 2. HSV Vector Exemplary Embodiment

In some embodiments, the haplotype modification is facilitated by employing a herpes simplex virus based viral technology, for example, and including the herpes simplex virus platform, as described in International Patent Publication Nos. WO 2019/243847 and/or WO 2017/118865, incorporated herein by reference in its entirety. In some embodiments, the viral vector is a herpes simplex virus vector. In some embodiments, the viral vector is a herpes simplex virus vector comprising haplotype modifying sequences. In some embodiments, when the viral vector is a herpes simplex virus vector, the administration route is intratumoral.

In some embodiments, the herpes simplex virus can be wild type (i.e., unaltered from the parental virus species), or with gene disruptions or gene additions. In some embodiments, the viral vector for use with the present invention comprises viruses expressing a fusogenic protein and at least one immune stimulatory molecule. In some embodiments, the viral vector for provides for direct oncolytic effects, viral replication and spread through tumors, mediated by the fusogenic protein, which (i) increases the amount of tumor antigens, including neoantigens, which are released for the induction of an antitumor immune response; and (ii) enhances the expression of the virus-encoded immune stimulatory molecule(s). In some embodiments, the fusogenic protein is the glycoprotein from gibbon ape leukemia virus (GALV) and has the R transmembrane peptide mutated or removed (GALV-R-).

In some embodiments, the viral vector is a herpes simplex virus (HSV). In some embodiments, the viral vector is a HSV1. In some embodiments, the viral vector is strain RH018A having the provisional accession number ECCAC 16121904; strain RH004A having the provisional accession number ECC AC 16121902; strain RH031A having the provisional accession number ECCAC 16121907; strain RH040B having the provisional accession number ECCAC 16121908; strain RH015A having the provisional accession number ECCAC 16121903; strain RH021A having the provisional accession number ECCAC 16121905; strain RH023A having the provisional accession number ECC AC 16121906; or strain RH047A having the provisional accession number ECCAC 16121909. In some embodiments, the viral vector is strain RH018A having the provisional accession number EACC 16121904.

In some embodiments, the viral vector does not express functional ICP34.5, does not express functional ICP47; and/or expresses the US11 gene as an immediate early gene.

In some embodiments, the viral vector comprises a nucleic acid encoding for a fusogenic protein selected from the group consisting of vesicular stomatitis virus (VSV) G-protein, syncitin-1, syncitin-2, simian virss 5 (SV5) F-protein, measles viras (MV) H-protein, MV F-protein, respiratory syncytial viras (RSV) F-protein and a glycoprotein from gibbon ape leukemia virus (GALV), murine leukemia virus (MLV), Mason-Pfizer monkey viras (MPMV) or equine infectious anaemia virus (EIAV) from which the R peptide has been deleted.

In some embodiments, the viral vector is a herpes simplex virus (HSV), such as HSV1. The HSV typically does not express functional ICP34.5 and/or functional ICP47 and/or expresses the US11 gene as an immediate early gene. In some embodiments, the ICP34.5-encoding genes are mutated to confer selective oncolytic activity on the HSV. Mutations of the ICP34.5-encoding genes that prevent the expression of functional ICP34.5 are described in Chou et al. (1990) Science 250:1262-1266, Maclean et al. (1991) J. Gen. Virol. 72:631-639 and Liu et al. (2003) Gene Therapy 10:292-303, which are incorporated herein by reference. The ICP6-encoding gene and/or thymidine kinase-encoding gene may also be inactivated, as may other genes provided that such inactivation does not prevent the virus infecting or replicating in tumors. In some embodiments, the deletion of the ICP47-encoding gene in a manner that places the US 11 gene under the control of the immediate early promoter that normally controls expression of the ICP47 encoding gene leads to enhanced replication in tumors (see Liu et al, 2003, which is incorporated herein by reference).

The virus may be a strain of any virus species which may be used for the oncolytic treatment of cancer, including strains of herpes vifrus, pox virus, adenovirus, retrovirus, rhabdovirus, paramyxovirus or reovirus. The virus is preferably a herpes simplex virus (HSV), such as HSV1. The HSV typically does not express functional ICP34.5 and/or functional ICP47 and/or expresses the US 11 gene as an immediate early gene.

In some embodiments, the virus is a herpes virus (HSV), including strains of HSV 1 and/or HS V2,

In some embodiments, other mutations that place the US11 coding sequence, which is an HSV late gene, under the control of a promoter that is not dependent on viral replication may also be introduced into herpes virus. Such mutations allow expression of US 11 before HSV replication occurs and enhance viral replication in tumors. In particular, such mutations enhance replication of an HSV lacking functional ICP34.5-encoding genes.

In some embodiments, the HSV of the disclosure comprises a US 11 gene operably linked to a promoter, wherein the activity of the promoter is not dependent on viral replication. The promoter may be an immediate early (IE) promoter or a non-HSV promoter which is active in mammalian, preferably human, tumor cells. The promoter may, for example, be a eukaryotic promoter, such as a promoter derived from the genome of a mammal, preferably a human. The promoter may be a ubiquitous promoter (such as a promoter of b-actin or tubulin) or a cell-specific promoter, such as tumor-specific promoter. The promoter may be a viral promoter, such as the Moloney murine leukaemia virus long terminal repeat (MMLV LTR) promoter or the human or mouse cytomegalovirus (CMV) IE promoter. HSV immediate early (IE) promoters are well known in the art. The HSV IE promoter may be the promoter driving expression of ICPO, ICP4, ICP22, ICP27 or ICP47.

d. Myxoma Virus (MV)

### 1. MV Vector Exemplary Embodiment

In some embodiments, the haplotype modification is facilitated by employing a myoxoma virus based viral technology, for example, and including the myxoma virus platform, as described in International Patent Publication Nos. WO 2020/051248, incorporated herein by reference in its entirety. In some embodiments, the viral vector is a herpes simplex virus vector. In some embodiments, the viral vector is a myoxoma virus virus vector comprising haplotype modifying sequences. In some embodiments, when the viral vector is a myxoma virus, the administration route is systemic.

In some embodiments, the viral vector is a myxoma virus (MYXV) based vector. In some embodiments, the myxoma virus (MYXV) comprises a LIGHT (Lymphotoxins-like, exhibits inducible expression, and competes with HSV Glycoprotein D for Herpesvirus entry mediator (HVEM), a receptor expressed by T lymphocytes) sequence. In some embodiments, the myxoma virus comprises MYXV-LIGHT. In some embodiments, the LIGHT comprises a sequence from human LIGHT. In some embodiments, the LIGHT comprises a sequence that is at least 70% identical to any one of SEQ ID NOs: 13-15 (from WO 2020/051248), now 47-49 in the present application, and copied below:

| | |
|---|---|
| Human | MEESVVRPSVFVVDGQTDIPFTRLGRSHRRQSC |
| LIGHT [symbol] | SVARVGLGLLLLLMGAGLAVQGWFLLQLHWRLG |
| TNFSF14, | EMVTRLPDGPAGSWEQLIQERRSHEVNPAAHLT |
| isoform 1 | GANSSLTGSGGPLLWETQLGLAFLRGLSYHDGA |
| | LVVTKAGYYYIYSKVQLGGVGCPLGLASTTTHG |
| | LYKRTPRYPEELELLVSQQSPCGRATSSSRVWW |
| | DSSFLGGVVHLEAGEKVVVRVLDERLVRLRDGT |
| | RSYFGAFMV |
| Human | MEESVVRPSVPVVDGQTDIPFTRLGRSHRRQSC |
| LIGHT [symbol] | SVARDGPAGSWEQLIQERRSHEVNPAAHLTGAN |
| TNFSF14, | SSLTGSGGPLLWETQLGLAFLRGLSYHDGALVV |
| isoform 2 | TKAGYYYIYSKVQLGGVGCPLGLASTTTHGLYK |
| | RTPRYPEELELLVSQQSPCGRATSSSRVWWDSS |
| | FLGGVVHLEAGEKVVVRVLDERLVRLRDGTRSY |
| | FGAFMV |
| Mouse | MESVVQPSVFVVDGQTDIPFRRLEQNHRRRRCG |
| LIGHT [symbol] | TVQVSLALVLLLGAGLATQGWFLLRLHQRLGDI |
| TNFSF14 | VAHLPDGGKGSWEKLIQDQRSHQANPAAHLTGA |
| | NASLIGIGGPLLWETRLGLAFLRGLTYHDGALV |
| | TMEPGYYYVYSKVQLSGVGCPQGLANGLP [symbol] HGL |
| | YKRTSRYPKELELLVSRRSPCGRANSSRVWWDS |
| | SFLGGVVHLEAGEEVVVRVPGNRLVRPRDGTRS |
| | YFGAFMV |

[symbol]

indicates data missing or illegible when filed

In some embodiments, the LIGHT is between the M135 and M136 open reading frames of the myxoma virus genome. In some embodiments, the myxoma virus comprises MYXV-FLuc-huLIGHT-TdTomato. In some embodiments, the myxoma virus comprises MYXV-Decorin. In some embodiments, a LIGHT transgene comprises a sequence from a mammalian LIGHT gene. In some embodiments, a LIGHT transgene comprises a sequence from a mouse LIGHT gene (mLIGHT). In some embodiments, a LIGHT transgene comprises a sequence from a human LIGHT gene (huLIGHT). In some embodiments, a LIGHT transgene encodes a product that is secreted. In some embodiments, a LIGHT transgene encodes a product that localizes to the cell surface (e.g., comprises a transmembrane domain). In some embodiments, a LIGHT gene comprises a sequence from any one of SEQ ID NOs: 46-48, as provided above.

In some embodiments, the myxoma virus comprises a deletion or disruption of one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD. In some embodiments, the myxoma virus comprises a deletion of M135.

### II. Solid Tumors for Treatment

In some embodiments, the methods described herein are useful in the treatment of solid cancers or tumors. The term “cancer” generally refers to tumors, including both primary and metastasized tumors. In some embodiments, the tumor is a solid tumor. As part of the methods provided herein, the methods find use in, for example, inhibiting solid cancer growth, including complete cancer remission, for inhibiting cancer metastasis, and for promoting cancer resistance, as well as for enhancing patient survival. The term “cancer growth” generally refers to any one of a number of indices that suggest change within the cancer to a more developed form. Thus, indices for measuring an inhibition of cancer growth include but are not limited to a decrease in cancer cell survival, a decrease in tumor volume or morphology (for example, as determined using computed tomographic (CT), sonography, or other imaging method), a delayed tumor growth, a destruction of tumor vasculature, improved performance in delayed hypersensitivity skin test, an increase in the activity of cytolytic T-lymphocytes, and a decrease in levels of tumor-specific antigens, as well as increases in patient survival outcomes.

In some embodiments, the cancer comprises a solid tumor, for example, a carcinoma, a sarcoma, and/or a lymphoma. Carcinomas include malignant neoplasms derived from epithelial cells which infiltrate, for example, invade, surrounding tissues and give rise to metastases. Adenocarcinomas are carcinomas derived from glandular tissue, or from tissues that form recognizable glandular structures. Another broad category of cancers includes sarcomas and fibrosarcomas, which are tumors whose cells are embedded in a fibrillar or homogeneous substance, such as embryonic connective tissue.

In some embodiments, the solid tumor is from a cancer or carcinoma of the bladder, uterine cervix, stomach, breast, lung, colon, rectum, skin, melanoma, gastrointestinal tract, urinary tract, or pancreas.

In some embodiments, carcinomas include but are not limited to adrenocortical, acinar, acinic cell, acinous, adenocystic, adenoid cystic, adenoid squamous cell, cancer adenomatosum, adenosquamous, adnexel, cancer of adrenal cortex, adrenocortical, aldosterone-producing, aldosterone-secreting, alveolar, alveolar cell, ameloblastic, ampullary, anaplastic cancer of thyroid gland, apocrine, basal cell, basal cell, alveolar, comedo basal cell, cystic basal cell, morphea-like basal cell, multicentric basal cell, nodulo-ulcerative basal cell, pigmented basal cell, sclerosing basal cell, superficial basal cell, basaloid, basosquamous cell, bile duct, extrahepatic bile duct, intrahepatic bile duct, bronchioalveolar, bronchiolar, bronchioloalveolar, bronchoalveolar, bronchoalveolar cell, bronchogenic, cerebriform, cholangiocelluarl, chorionic, choroids plexus, clear cell, cloacogenic anal, colloid, comedo, corpus, cancer of corpus uteri, cortisol-producing, cribriform, cylindrical, cylindrical cell, duct, ductal, ductal cancer of the prostate, ductal cancer in situ (DCIS), eccrine, embryonal, cancer en cuirasse, endometrial, cancer of endometrium, endometroid, epidermoid, cancer ex mixed tumor, cancer ex pleomorphic adenoma, exophytic, fibrolamellar, cancer fibro'sum, follicular cancer of thyroid gland, gastric, gelatin form, gelatinous, giant cell, giant cell cancer of thyroid gland, cancer gigantocellular (including gigantocellular reticular nucleus), glandular, granulose cell, hepatocellular, Hurthle cell, hypernephroid, infantile embryonal, islet cell carcinoma, inflammatory cancer of the breast, cancer in situ, intraductal, intraepidermal, intraepithelial, juvenile embryonal, Kulchitsky-cell, large cell, leptomeningeal, lobular, infiltrating lobular, invasive lobular, lobular cancer in situ (LCIS), lymphoepithelial, cancer medullare, medullary, medullary cancer of thyroid gland, medullary thyroid, melanotic, meningeal, Merkel cell, metatypical cell, micropapillary, mucinous, cancer muciparum, nasopharyngeal, neuroendocrine cancer of the skin, non-small cell lung cancer (NSCLC), oat cell, cancer ossificans, osteoid, Paget's, papillary, papillary cancer of thyroid gland, periampullary, preinvasive, prickle cell, renal cell, scar, schistosomal bladder, Schneiderian, scirrhous, sebaceous, signet-ring cell, small cell lung cancer (SCLC), spindle cell, cancer spongiosum, squamous, squamous cell, terminal duct, anaplastic thyroid, follicular thyroid, medullary thyroid, papillary thyroid, trabecular cancer of the skin, transitional cell, tubular, undifferentiated cancer of thyroid gland, uterine corpus, verrucous, squamous cell (including head and neck), esophageal squamous cell, and/or oral cancers and carcinomas.

In some embodiments, the sarcomas include but are not limited to adipose, alveolar soft part, ameloblastic, avian, botryoid, sarcoma botryoides, chicken, chloromatous, chondroblastic, clear cell sarcoma of kidney, embryonal, endometrial stromal, epithelioid, Ewing's, fascial, fibroblastic, fowl, giant cell, granulocytic, hemangioendothelial, Hodgkin's, idiopathic multiple pigmented hemorrhagic, immunoblastic sarcoma of B cells, immunoblastic sarcoma of T cells, Jensen's, Kaposi's, Kupffer cell, leukocytic, lymphatic, melanotic, mixed cell, multiple, lymphangioma, idiopathic hemorrhagic, multipotential primary sarcoma of bone, osteoblastic, osteogenic, parosteal, polymorphous, pseudo-kaposi, reticulum cell, reticulum cell sarcoma of the brain, rhabdomyosarcoma, soft tissue, spindle cell, synovial, telangiectatic, sarcoma (osteosarcoma)/malignant fibrous histiocytoma of bone, and/or soft tissue sarcomas.

In some embodiments, lymphomas include but are not limited to AIDS-related, non-Hodgkin's, Hodgkin's, T-cell, T-cell leukemia/lymphoma, African, B-cell, B-cell monocytoid, bovine malignant, Burkitt's, centrocytic, lymphoma cutis, diffuse, diffuse, large cell, diffuse, mixed small and large cell, diffuse, small cleaved cell, follicular, follicular center cell, follicular, mixed small cleaved and large cell, follicular, predominantly large cell, follicular, predominantly small cleaved cell, giant follicle, giant follicular, granulomatous, histiocytic, large cell, immunoblastic, large cleaved cell, large nucleated cell, Lennert's, lymphoblastic, lymphocytic, intermediate lymphocytic, intermediately differentiated lymphocytic, plasmacytoid, poorly differentiated lymphocytic, small lymphocytic, well differentiated lymphocytic, MALT, mantle cell, mantle zone, marginal zone, Mediterranean lymphoma, mixed lymphocytic-histiocytic, nodular, plasmacytoid, pleomorphic, primary central nervous system, primary effusion, small b-cell, small cleaved cell, small nucleated cell, T-cell lymphomas, convoluted T-cell, cutaneous T-cell, small lymphocytic T-cell, undefined lymphoma, u-cell, undifferentiated, AIDS-related, cutaneous T-cell, effusion (body cavity based), thymic lymphoma, and/or cutaneous T cell lymphomas.

In some embodiments, gastrointestinal solid cancers that may be targeted include extrahepatic bile duct cancer, colon cancer, colon and rectum cancer, colorectal cancer, gallbladder cancer, gastric (stomach) cancer, gastrointestinal carcinoid tumor, gastrointestinal carcinoid tumors, gastrointestinal stromal tumors, bladder cancers, islet cell carcinoma (endocrine pancreas), pancreatic cancer, islet cell pancreatic cancer, prostate cancer rectal cancer, salivary gland cancer, small intestine cancer, colon cancer, and/or polyps associated with colorectal neoplasia.

In some embodiments, lung and respiratory solid cancers include but are not limited to bronchial adenomas/carcinoids, esophagus cancer esophageal cancer, esophageal cancer, hypopharyngeal cancer, laryngeal cancer, hypopharyngeal cancer, lung carcinoid tumor, non-small cell lung cancer, small cell lung cancer, small cell carcinoma of the lungs, mesothelioma, nasal cavity and paranasal sinus cancer, nasopharyngeal cancer, nasopharyngeal cancer, oral cancer, oral cavity and lip cancer, oropharyngeal cancer, paranasal sinus and nasal cavity cancer, and/or pleuropulmonary blastoma.

In some embodiments, urinary tract and reproductive cancers include but are not limited to cervical cancer, endometrial cancer, ovarian epithelial cancer, extragonadal germ cell tumor, extracranial germ cell tumor, extragonadal germ cell tumor, ovarian germ cell tumor, gestational trophoblastic tumor, spleen, kidney cancer, ovarian cancer, ovarian epithelial cancer, ovarian germ cell tumor, ovarian low malignant potential tumor, penile cancer, renal cell cancer (including carcinomas), renal cell cancer, renal pelvis and ureter (transitional cell cancer), transitional cell cancer of the renal pelvis and ureter, gestational trophoblastic tumor, testicular cancer, ureter and renal pelvis, transitional cell cancer, urethral cancer, endometrial uterine cancer, uterine sarcoma, vaginal cancer, vulvar cancer, ovarian carcinoma, primary peritoneal epithelial neoplasms, cervical carcinoma, uterine cancer and solid tumors in the ovarian follicle), superficial bladder tumors, invasive transitional cell carcinoma of the bladder, and/or muscle-invasive bladder cancer.

In some embodiments, the skin cancers and melanomas (as well as non-melanomas) include but are not limited to cutaneous t-cell lymphoma, intraocular melanoma, tumor progression of human skin keratinocytes, basal cell carcinoma, and squamous cell cancer. Liver cancers that may be targeted include extrahepatic bile duct cancer, and hepatocellular cancers. Eye cancers that may be targeted include intraocular melanoma, retinoblastoma, and intraocular melanoma Hormonal cancers that may be targeted include: parathyroid cancer, pineal and supratentorial primitive neuroectodermal tumors, pituitary tumor, thymoma and thymic carcinoma, thymoma, thymus cancer, thyroid cancer, cancer of the adrenal cortex, and/or ACTH-producing tumors.

In some embodiments of the methods or uses described herein, the administration of the TCR-T inhibits solid tumor growth. In some embodiments of the methods or uses described herein, the administration of the TCR-T inhibits solid tumor growth by at least 10%, at least 15%, at least 20%, at least 25%, at least 30%, at least 35%, at least 40%, at least 50%, at least 55%, at least 60%, at least 70%, at least 75%, at least 80%, at least 85%, at least 90%, at least 95%, or at least 99%. In some embodiments of the methods or uses described herein, the administration of the TCR-T inhibits solid tumor growth by at least 1-fold, at least 2-fold, at least 3-fold, at least 4-fold, at least 5-fold, at least 6-fold, at least 7-fold, at least 8-fold, at least 9-fold, or at least 10-fold. In some embodiments, solid tumor growth is measured within 7 days, within 14 days, within 21 days, within 30 days, within 40 days, within 50 days, within 60 days, within 70 days, within 80 days, within 3 months, within 4 months, within 5 months, within 6 months, within 1 year, or more than 1 year after injection with TCR-T cells.

In some embodiments, the TCR-T comprises TCR-T cells, including an infusion of TCR-T cells. In some embodiments, the TCR-T comprises TCR-T cells, including an infusion of TCR-T cells subsequently to genetically modifying the haplotype of the population of tumor cells. In some embodiments, the TCR-T comprises TCR-T cells, including an infusion of TCR-T cells subsequently to genetically modifying the HLA haplotype of the population of tumor cells.

### III. TCR Sequences and Enhanced T-Cell Activity

As described herein, the present invention provide methods, nucleic acids and vectors related to genetically modifying a population of tumor cells to render the tumor cells more susceptible to TCR therapy (TCR-T).

In some embodiments, the TCR-T is administered subsequently to genetically modifying the population of tumor cells to express a tumor haplotype different from the tumor haplotype endogenous to the population of tumor cells.

In some embodiments, the TCR-T comprises a restricted and/or targeted TCR. In some embodiments, the restricted and/or targeted TCR is encoded by a nucleic acid encoding a TCR α-chain and a nucleic acid encoding a TCR β-chain.

In some embodiments, the restricted and/or targeted TCR is encoded by a nucleic acid encoding a TCR α-chain and a nucleic acid encoding a TCR β-chain are contained under separate open reading frames.

In certain embodiments, the nucleic acid encoding a TCR α-chain and the nucleic acid encoding a TCR β-chain are contained in a single open reading frame, wherein the single open reading frame further comprises a polynucleotide encoding a self-cleaving peptide disposed between the α-chain-encoding polynucleotide and the 0-chain-encoding polynucleotide. In some embodiments, the restricted and/or targeted TCR is encoded by a nucleic acid encoding a TCR a-chain and a nucleic acid encoding a TCR β-chain are encoded by a single vector. In some embodiments, the restricted and/or targeted TCR is encoded by a nucleic acid encoding a TCR a-chain and a nucleic acid encoding a TCR β-chain are encoded by separate vectors.

In some embodiments, the TCR-engineered T cells (TCR-T) therapy targets a TCR with a specific antigen specificity. In some embodiments, the TCR has specificity for TCR having antigenic specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-All, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

In some embodiments, the TCR has comprise a TCR α-chain and a TCR β-chain a has specificity for TCR having antigenic specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

In some embodiments of the method or use as described herein, the TCR-T therapy comprises a TCR having antigenic specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

In some embodiments of the method or use as described herein, the TCR-T therapy comprises a TCR having antigenic specificity for HERV-E, KK-LC-1, or NY-ESO-1.

In some embodiments of the method or use as described herein, the TCR targets KK-LC-1. In some embodiments, the TCR comprises a KK-LC-i-TCR sequence. In some embodiments of the method or use as described herein, the TCR-T therapy comprises a TCR having antigenic specificity for KK-LC-1. In some embodiments of the method or use as described herein, one or more vectors comprise a KK-LC-i-TCR sequence. In some embodiments of the method or use as described herein, a vector comprises a KK-LC-I-TCR beta sequence. In some embodiments, a vector comprises a KK-LC-I-TCR alpha sequence. In some embodiments of the method or use as described herein, a vector comprises a KK-LC-I-TCR beta sequence and a KK-LC-I-TCR alpha sequence. In some embodiments of the method or use as described herein, the TCR-T therapy comprises a TCR having antigenic specificity for Kita-Kyushu Lung Cancer Antigen-1₅₂₋₆₀ (KK-LC-1₅₂₋₆₀). In some embodiments of the method or use as described herein, the KK-LC-1₅₂₋₆₀ comprises the amino acid sequence NTDNNLAVY (SEQ ID NO:11).

In some embodiments of the method or use as described herein, the TCR targets the HERV-E. In some embodiments of the method or use as described herein, the TCR comprises a HERV-E-TCR sequence. In some embodiments of the method or use as described herein, the TCR-T therapy comprises a TCR having antigenic specificity for HERV-E. In some embodiments of the method or use as described herein, one or more vectors comprise a HERV-E-TCR sequence. In some embodiments of the method or use as described herein, a vector comprises a HERV-E-TCR beta sequence. In some embodiments, a vector comprises a HERV-E-TCR alpha sequence. In some embodiments of the method or use as described herein, a vector comprises a HERV-E-TCR beta sequence and a HERV-E-TCR alpha sequence. In some embodiments of the method or use as described herein, HERV-E comprises the amino acid sequence ATFLGSLTWK (SEQ ID NO:22).

In some embodiments of the method or use as described herein, the TCR targets the NY-ESO-1. In some embodiments of the method or use as described herein, the TCR comprises a NY-ESO-1-TCR sequence. In some embodiments of the method or use as described herein, the TCR-T therapy comprises a TCR having antigenic specificity for NY-ESO-1. In some embodiments of the method or use as described herein, one or more vectors comprise a NY-ESO-1-TCR sequence. I n some embodiments of the method or use as described herein, a vector comprises a NY-ESO-1-TCR beta sequence. In some embodiments, a vector comprises a NY-ESO-1-TCR alpha sequence. In some embodiments of the method or use as described herein, a vector comprises a NY-ESO-1-TCR beta sequence and a NY-ESO-1-TCR alpha sequence. In some embodiments of the method or use as described herein, the TCR-T therapy comprises a TCR having antigenic specificity for NY-ESO-1157-165. In some embodiments of the method or use as described herein, the NY-ESO-1157-165 comprises the amino acid sequence SLLMWITQC (SEQ ID NO:33).

In some embodiments of the method or use as described herein, the TCR comprises the amino acid sequences of SEQ ID NO: 5 and/or 10.

In some embodiments of the method or use as described herein, the TCR comprises the amino acid sequences of SEQ ID NO: 16 and/or 21.

In some embodiments of the method or use as described herein, the TCR comprises the amino acid sequences of SEQ ID NO: 27 and/or 32.

In some embodiments of the method or use as described herein, the TCR comprises the amino acid sequences of SEQ ID NO: 38 and/or 43.

In some embodiments of the method or use as described herein, the TCR comprises nucleic acids encoding a TCR beta chain and a TCR alpha chain, wherein the nucleotide sequence encoding the beta chain is positioned 5′ of the nucleotide sequence encoding the alpha chain.

In some embodiments of the method or use as described herein, the TCR-T comprises a T-cell receptor α-chain comprising an amino acid sequence encoded by a nucleic acid sequence comprising an αCDR1, αCDR2, and αCDR3; a T-cell receptor β-chain comprising an amino acid sequence encoded by a nucleic acid sequence comprising βCDR1, βCDR2, and βCDR3; or both. In some embodiments of the method or use as described herein, the TCR-T comprises a T-cell receptor α-chain comprising an amino acid sequence encoded by a nucleic acid sequence comprising an αCDR1, αCDR2, and αCDR3 and a T-cell receptor β-chain comprising an amino acid sequence encoded by a nucleic acid sequence comprising βCDR1, βCDR2, and βCDR3.

In some embodiments of the method or use as described herein, the TCR-T comprises a T-cell receptor α-chain comprising an αCDR1, αCDR2, and αCDR3 with specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6. In some embodiments of the method or use as described herein, the TCR-T comprises a T-cell receptor β-chain comprising an βCDR1, βCDR2, and βCDR3 with specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

In some embodiments of the method or use as described herein, the TCR-T comprises a T-cell receptor α-chain comprising an amino acid sequence encoded by a nucleic acid sequence comprising an αCDR1, αCDR2, and αCDR3 with specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6. In some embodiments of the method or use as described herein, the TCR-T comprises a T-cell receptor j-chain comprising an amino acid sequence encoded by a nucleic acid sequence comprising an βCDR1, βCDR2, and βCDR3 with specificity for KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-All, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 6, 17, 28 or 39; a T cell receptor β-chain comprising an amino acid sequence encoded by a nucleic acid sequence comprising at least 80% sequence identity to the nucleic acid sequence of SEQ ID NO: 1, 12, 23, or 34; or both. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 6 and a T cell receptor β-chain comprising an amino acid sequence encoded by a nucleic acid sequence comprising at least 80% sequence identity to the nucleic acid sequence of SEQ ID NO: 1. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 17 and a T cell receptor β-chain comprising an amino acid sequence encoded by a nucleic acid sequence comprising at least 80% sequence identity to the nucleic acid sequence of SEQ ID NO: 12. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 28 and a T cell receptor β-chain comprising an amino acid sequence encoded by a nucleic acid sequence comprising at least 80% sequence identity to the nucleic acid sequence of SEQ ID NO: 23. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 39 and a T cell receptor β-chain comprising an amino acid sequence encoded by a nucleic acid sequence comprising at least 80% sequence identity to the nucleic acid sequence of SEQ ID NO: 34. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 6, 17, 28 or 39 and comprising an αCDR1, αCDR2, and αCDR3 from SEQ ID NO: 6, 17, 28 or 39; a T cell receptor β-chain comprising an amino acid sequence encoded by a nucleic acid sequence comprising at least 80% sequence identity to the nucleic acid sequence of SEQ ID NO: 1, 12, 23, or 34 and a βCDR1, βCDR2, and βCDR3 from SEQ ID NO: 1, 12, 23, or 34; or both. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 6 and comprising an αCDR1, αCDR2, and αCDR3 from SEQ ID NO: 6, and a T cell receptor β-chain comprising an amino acid sequence encoded by a nucleic acid sequence comprising at least 80% sequence identity to the nucleic acid sequence of SEQ ID NO: 1 and a βCDR1, βCDR2, and βCDR3 from SEQ ID NO: 1. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 17 and comprising an αCDR1, αCDR2, and αCDR3 from SEQ ID NO: 17, and a T cell receptor β-chain comprising an amino acid sequence encoded by a nucleic acid sequence comprising at least 80% sequence identity to the nucleic acid sequence of SEQ ID NO: 12 and a βCDR1, βCDR2, and βCDR3 from SEQ ID NO: 12. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 28 and comprising an αCDR1, αCDR2, and αCDR3 from SEQ ID NO: 28, and a T cell receptor β-chain comprising an amino acid sequence encoded by a nucleic acid sequence comprising at least 80% sequence identity to the nucleic acid sequence of SEQ ID NO: 23 and a βCDR1, βCDR2, and βCDR3 from SEQ ID NO: 23. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising at least 80% sequence identity to the nucleic acid sequence of SEQ ID NO: 39 and comprising an αCDR1, αCDR2, and αCDR3 from SEQ ID NO: 39; a T cell receptor β-chain comprising an amino acid sequence encoded by a nucleic acid sequence comprising at least 80% sequence identity to the nucleic acid sequence of SEQ ID NO: 34 and a βCDR1, βCDR2, and βCDR3 from SEQ ID NO: 34. In some embodiments of the method or use as described herein, the sequence identity is at least 85%, at least 90%, at least 95%, at least 98%, or at least 99%, or in some embodiments, 100%.

In some embodiments of the method or use as described herein, the TCR-T comprises a T-cell receptor α-chain comprising an amino acid sequence encoded by a nucleic acid sequence comprising the αCDR1, αCDR2, and αCDR3 from a sequence selected from the group consisting of SEQ ID NO: 6, 17, 28 or 39; a T cell receptor β-chain comprising an amino acid sequence encoded by a nucleic acid sequence comprising the βCDR1, βCDR2, and βCDR3 from a sequence selected from the group consisting of SEQ ID NO: 1, 12, 23, or 34; or both.

In some embodiments of the method or use as described herein, the TCR-T comprises a T-cell receptor α-chain comprises the αCDR1, αCDR2, and αCDR3 from a sequence selected from the group consisting of SEQ ID NO: 5, 16, 27, or 38; a T-cell receptor β-chain comprises the βCDR1, βCDR2, and βCDR3 from a sequence selected from the group consisting of SEQ ID NO: 10, 21, 32, or 43; or both.

In some embodiments of the method or use as described herein, the TCR-T comprises a T-cell receptor α-chain comprises the αCDR1, αCDR2, and αCDR3 from SEQ ID NO: 5 and a T-cell receptor β-chain comprises the βCDR1, βCDR2, and βCDR3 from SEQ ID NO: 10.

In some embodiments of the method or use as described herein, the TCR-T comprises a T-cell receptor α-chain comprises the αCDR1, αCDR2, and αCDR3 from SEQ ID NO: 16 and a T-cell receptor β-chain comprises the βCDR1, βCDR2, and βCDR3 from SEQ ID NO: 21.

In some embodiments of the method or use as described herein, the TCR-T comprises a T-cell receptor α-chain comprises the αCDR1, αCDR2, and αCDR3 from SEQ ID NO: 27 and a T-cell receptor β-chain comprises the βCDR1, βCDR2, and βCDR3 from SEQ ID NO: 32.

In some embodiments of the method or use as described herein, the TCR-T comprises a T-cell receptor α-chain comprises the αCDR1, αCDR2, and αCDR3 from SEQ ID NO: 38 and a T-cell receptor β-chain comprises the βCDR1, βCDR2, and βCDR3 from SEQ ID NO: 43.

In some embodiments of the method or use as described herein, the TCR-T comprises a T-cell receptor α-chain comprising an amino acid sequence encoded by a nucleic acid sequence of SEQ ID NO: 5, 16, 27, or 38; a T-cell receptor β-chain comprising an amino acid sequence encoded by a nucleic acid sequence of SEQ ID NO: 10, 21, 32, or 43; or both.

In some embodiments of the method or use as described herein, the TCR-T comprises a T-cell receptor α-chain comprising an amino acid sequence encoded by a nucleic acid sequence of SEQ ID NO: 5 and a T-cell receptor β-chain comprising an amino acid sequence encoded by a nucleic acid sequence of SEQ ID NO: 10.

In some embodiments of the method or use as described herein, the TCR-T comprises a T-cell receptor α-chain comprising an amino acid sequence encoded by a nucleic acid sequence of SEQ ID NO: 16 and a T-cell receptor 0-chain comprising an amino acid sequence encoded by a nucleic acid sequence of SEQ ID NO: 21.

In some embodiments of the method or use as described herein, the TCR-T comprises a T-cell receptor α-chain comprising an amino acid sequence encoded by a nucleic acid sequence of SEQ ID NO: 27 and a T-cell receptor β-chain comprising an amino acid sequence encoded by a nucleic acid sequence of SEQ ID NO: 32.

In some embodiments of the method or use as described herein, the TCR-T comprises a T-cell receptor α-chain comprising an amino acid sequence encoded by a nucleic acid sequence of SEQ ID NO: 38 and a T-cell receptor β-chain comprising an amino acid sequence encoded by a nucleic acid sequence of SEQ ID NO: 43.

In some embodiments of the method or use as described herein, the vector comprises a TCR-T comprising a T-cell receptor α-chain comprising an amino acid sequence encoded by a nucleic acid sequence comprising an αCDR1, αCDR2, and αCDR3 and a T-cell receptor β-chain comprising an amino acid sequence encoded by a nucleic acid sequence comprising βCDR1, βCDR2, and βCDR3. In some embodiments of the method or use as described herein, one vector comprises a TCR-T comprising a T-cell receptor α-chain comprising an amino acid sequence encoded by a nucleic acid sequence comprising an αCDR1, αCDR2, and αCDR3 and a second vector comprises a T-cell receptor β-chain comprising an amino acid sequence encoded by a nucleic acid sequence comprising βCDR1, βCDR2, and βCDR3.

In some embodiments of the method or use as described herein, the vector comprises the nucleic acid sequence of SEQ ID NO: 1 and the nucleic acid sequence of SEQ ID NO: 6, or comprises a nucleic acid encoding for the amino sequence of SEQ ID NO: 5 and a nucleic acid encoding for the amino sequence of SEQ ID NO: 10.

In some embodiments of the method or use as described herein, the vector comprises the nucleic acid sequence of SEQ ID NO: 12 and the nucleic acid sequence of SEQ ID NO: 17, or comprises a nucleic acid encoding for the amino sequence of SEQ ID NO: 16 and a nucleic acid encoding for the amino sequence of SEQ ID NO: 21.

In some embodiments of the method or use as described herein, the vector comprises the nucleic acid sequence of SEQ ID NO: 23 and the nucleic acid sequence of SEQ ID NO: 28, or comprises a nucleic acid encoding for the amino sequence of SEQ ID NO: 27 and a nucleic acid encoding for the amino sequence of SEQ ID NO: 32.

In some embodiments of the method or use as described herein, the vector comprises the nucleic acid sequence of SEQ ID NO: 34 and the nucleic acid sequence of SEQ ID NO: 39, or comprises a nucleic acid encoding for the amino sequence of SEQ ID NO: 38 and a nucleic acid encoding for the amino sequence of SEQ ID NO: 43.

In some embodiments of the method or use as described herein, the vector comprises a nucleic acid sequence encoding the βCDR1, βCDR2, and βCDR3 of SEQ ID NO: 1 and a nucleic acid sequence encoding the αCDR1, αCDR2, and αCDR3 of SEQ ID NO: 6.

In some embodiments of the method or use as described herein, the vector comprises a nucleic acid sequence encoding the βCDR1, βCDR2, and βCDR3 of SEQ ID NO: 12 and a nucleic acid sequence encoding the αCDR1, αCDR2, and αCDR3 of SEQ ID NO: 17.

In some embodiments of the method or use as described herein, the vector comprises a nucleic acid sequence encoding the βCDR1, βCDR2, and βCDR3 of SEQ ID NO: 23 and a nucleic acid sequence encoding the αCDR1, αCDR2, and αCDR3 of SEQ ID NO: 28.

In some embodiments of the method or use as described herein, the vector comprises a nucleic acid sequence encoding the βCDR1, βCDR2, and βCDR3 of SEQ ID NO: 34 and a nucleic acid sequence encoding the αCDR1, αCDR2, and αCDR3 of SEQ ID NO: 39.

In some embodiments of the method or use as described herein, the method comprises genetically modifying the population of tumor cells to increase expression of a chemokine at the tumor site. Chemokines are chemical messenger proteins that are able to increase immune cell activity in a specific location by regulating differentiation and migration of immune cells. CXCL9, CXCL10, and CXCL 11 are examples of chemokines that regulate immune cell migration, differentiation, and activation (Lim, R. J., et al., 2024, Cell Reports Medicine, 5: 101479). They appear to signal through C-X-C motif chemokine receptor 3 (CXCR3) to facilitate effector T cell infiltration into the tumor. The CXCL9/CXCL10/CXCR3 signaling cascade is also required for optimal T cell activation.

In some embodiments of the method or use as described herein, the increased expression of a chemokine at the tumor site leads to increased number or activity of T cells at the tumor site. In some embodiments of the method or use as described herein, the chemokine is at least one of CXCL9, CXCL10, and CXCL11.

Increased expression of co-stimulatory molecules such as CD80 on tumor cells has also been found to promote T cell activation and tumor rejection (Vackova, J. et al., 2021, Cancers (Basel) 13:1935). In some embodiments of the method or use as described herein, the method comprises genetically modifying a tumor cell in the population of tumor cells to increase expression of CD80 on the tumor cell surface.

In some embodiments, the present invention provides a peptide comprising the amino acid sequence NTDNNLAVY (SEQ ID NO:11). In some embodiments, the present invention provides a peptide comprising the amino acid sequence ATFLGSLTWK (SEQ ID NO:22). In some embodiments, the present invention provides a peptide comprising the amino acid sequence SLLMWITQC (SEQ ID NO:33). In some embodiments of the method or use as described herein, the TCR-T therapy comprises a TCR having antigenic specificity for a peptide of NTDNNLAVY (SEQ ID NO:11), ATFLGSLTWK (SEQ ID NO:22), or SLLMWITQC (SEQ ID NO:33).

### EXAMPLES

### Example 1: A*11 Expression in Herv-E/A*11-Tumor Lines Confers Recognition by HERV-E-TCR Transduced T Cells

The present example provides for methods of enhancing diversity of HLA haplotype expression in tumors to broaden tumor cell susceptibility to TCR-T therapy.

The limitations of any TCR therapy are two-fold. First, the tumor cells must express the target peptide, here HERV-E. The correct HLA molecule that binds to the TCR and peptide must also be present on the target cells. We have shown the HERV-E-TCR to be very effective at recognizing tumor cells that naturally express both HERV-E and HLA-A*11. Whether expression of HLA-A*11 in tumor cells that are naturally HLA-A*11 negative but HERV-E positive is sufficient for tumor recognition remains an open question. The purpose of these experiments is to evaluate whether HERV-E-TCR transduced T cells would recognize tumors that express HERV-E but were not naturally A*11 positive after transduction with an A*11 expression vector.

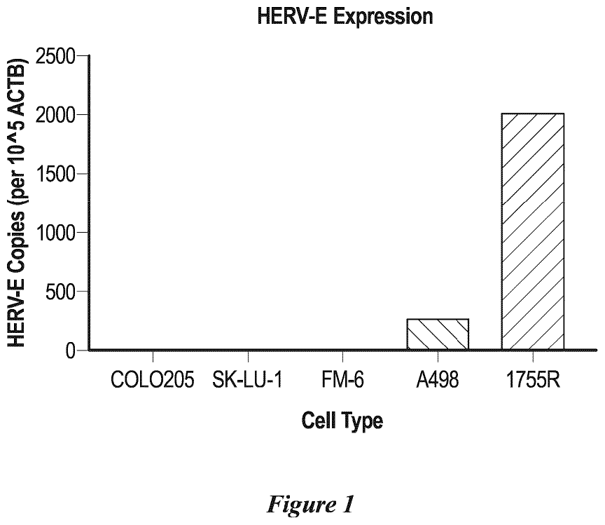

In normal tissues, expression of HERV-E is extremely low, falling below detectable limits. In some malignancies, especially those of the kidney and renal cells, expression of HERV-E becomes quite pronounced. Malignancies of the colon, lung, and skin, represented by COLO-205, SK-LU-1, and FM-6 respectively, show almost no expression HERV-E. Kidney and renal cell malignancies do show expression of HERV-E, here shown by A498 and 1755R (FIG. 1). The cells do not have the proper HLA molecules to be detected by the HERV-E TCR, however. A498 are HLA-A*02, and 1755R are HLA-A*02/ILA-A*31. In order to test whether introduction of the A*11 HLA into these cells would be sufficient to cause recognition with the HERV-E-TCR, these two cell lines were transduced with a retroviral vector containing an A*11 expression element. The pBABE retroviral vector also contained a puromycin resistance element, and after transduction the cells were selected for 10 days with the appropriate amount of puromycin.

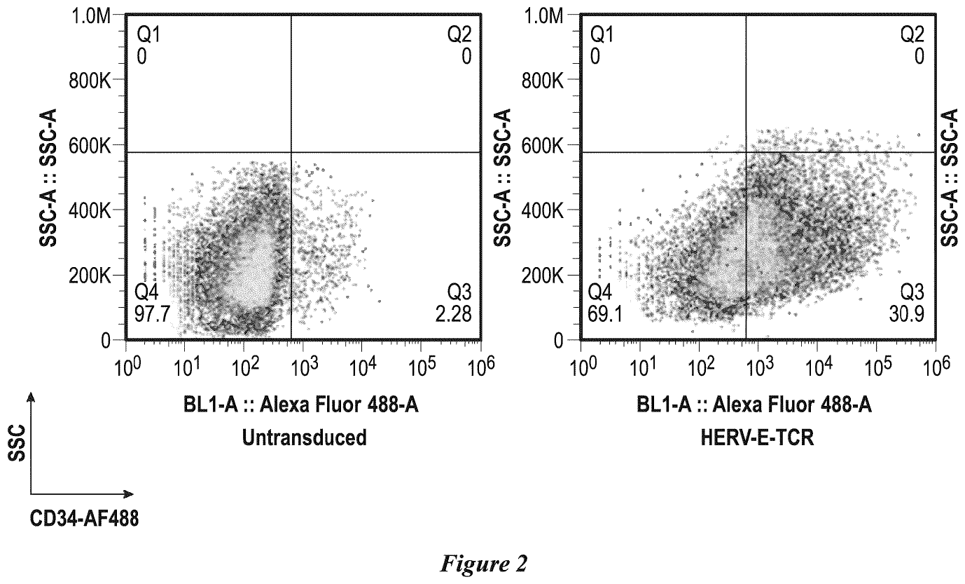

To make the effector cells, donor T cells were transduced with the HERV-E-TCR virus, which contains the Alpha and Beta TCR elements as well as a truncated CD34 element. After 4 days the cells were stained for CD34, which will detect how much of the population was transduced with the HERV-E-TCR. The result show that approximately 30% of the donor T cells are TCR positive (FIG. 2)

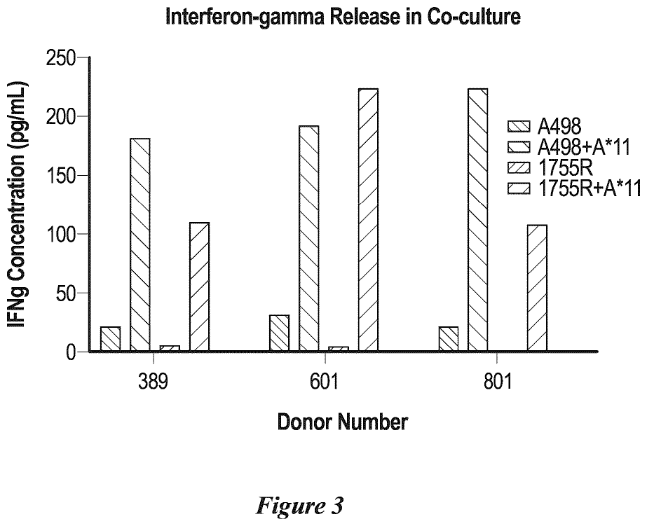

To test whether A*11 expression conferred target recognition to these transduced cells, a co-culture experiment was performed. The HERV-E-TCR transduced T cells were co-cultured with A498, A498+A*11, 1755R, and 1755R+A*11 cells. After 18 hours, the supernatant was collected and an ELSIA was performed on the supernatant for interferon-gamma (IFNγ). An increase in IFNγ release can be seen in both A*11 transduced cells, A498+A*11 and 1755R+A*11 (FIG. 3)

This data shows that the introduction of A*11 expression into tumors that are HERV-E positive but naturally HLA-A*11 negative leads to T cell recognition of these tumor cells. Recognition also seems to be effective at lower expression rates, such as A498 versus 1755R. If this is broadly applicable, this would allow for previous HLA-restricted TCRs to be used in combination with a tumor-targeting virus carrying the HLA-A*11 expression vector. This would not only limit cross-reactivity, as other patient cells would not carry the HLA-A*11 allele, but also allow for T cell therapies to be used in an HLA-independent manner.

### Example 2: A*01 Expression in KK-LC-1+/A*01-Tumor Lines Confers Recognition by KK-LC-1-TCR Transduced T Cells

The KK-LC-1-TCR has been shown to be very effective at recognizing KK-LC-1 positive tumors that naturally express the HLA-A*01 protein (A*01). The limitations of any TCR therapy are 2-fold: (1) expression of the target peptide, here KK-LC-1, and (2) expression of the correct HLA-A molecule that the TCR and peptide bind. Whether expression of the HLA molecule was sufficient to confer recognition in KK-LC-1 positive but A*01 negative cell lines is an open question. The purpose of these experiments was to evaluate whether KK-LC-1-TCR transduced T cells would recognize tumors that express KK-LC-1 but were not naturally A*01 positive after transduction with an A*01 expression vector.

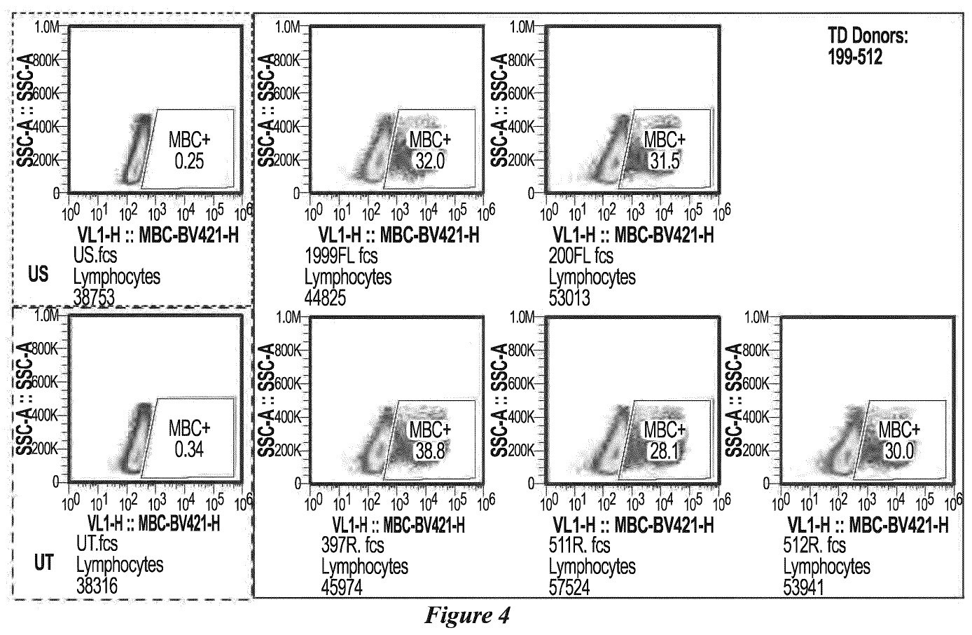

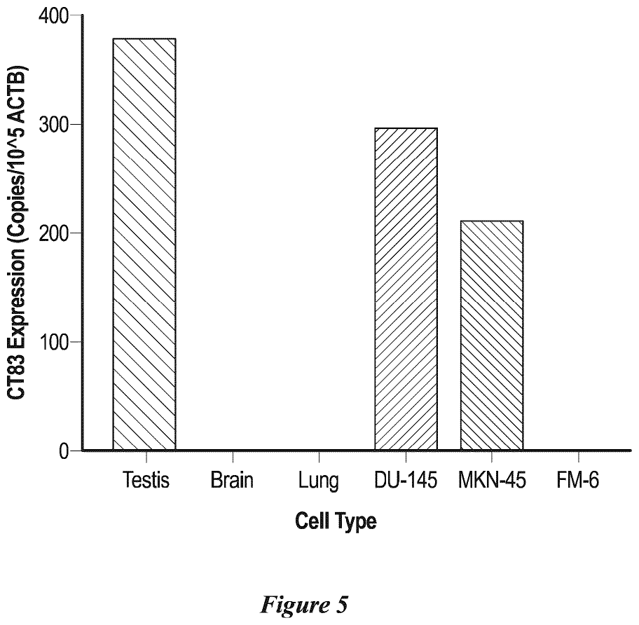

T cells from 5 healthy donors were transduced with the KK-LC-1-TCR retrovirus. After 4 days, the cells were stained with an antibody that binds the mouse T-cell receptor beta constant region. This region is only present on the KK-LC-1-TCR and will not be detected on Untransduced cells. T cells show transduction rates of approximately 30% across all donors (FIG. 4).

In non-cancer cells, expression of KK-LC-1 (CT83) is restricted to the immune privileged areas of the testis (FIG. 5). In cancer cells, aberrant expression of these testis restricted antigens leads to cancer-testis antigens that are targetable by immune therapy. Two such lines, DU-145 and MKN-45, show expression of CT83, while other lines show no expression, such as FM-6 (FIG. 5). Target cells were made by transducing two KK-LC-1 positive lines, DU-145 and MKN-45, with a pBABE retroviral construct containing a puromycin resistance cassette and an HLA-A*01 encoding element. DU-145 naturally express HLA-A*03 and HLA-A*33, and MKN-45 naturally express HLA-A*24. The cells were transduced with the pBABE construct and then treated with the appropriate amount of puromycin for 10 days to select for a puromycin resistant population which are KK-LC-1 positive and A*01 positive.

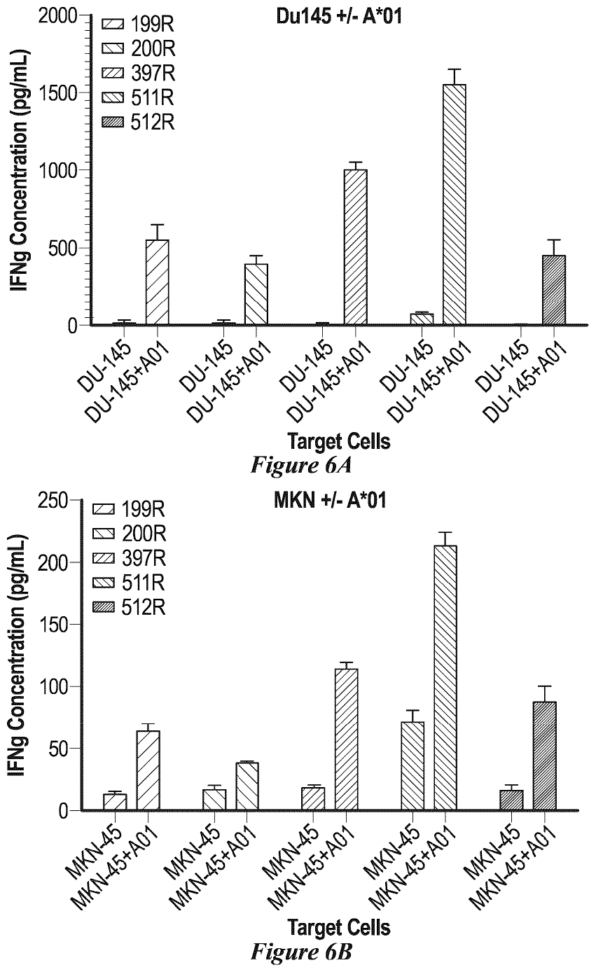

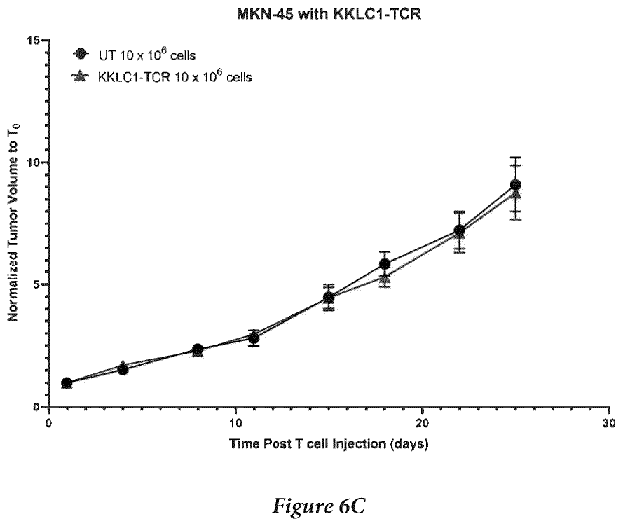

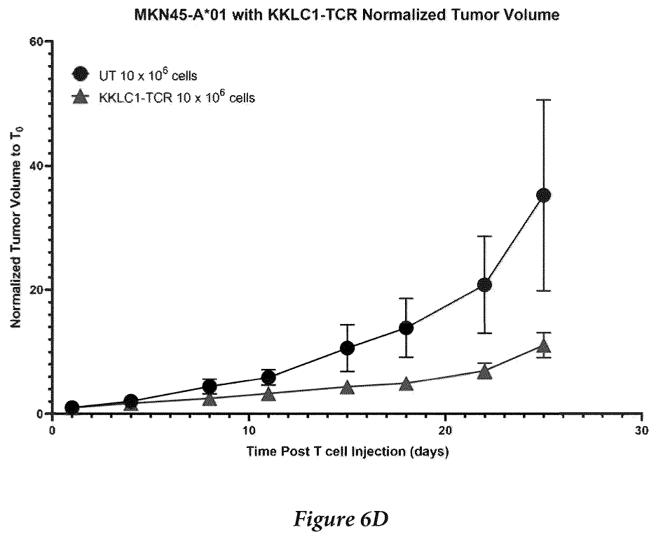

To test whether A*01 expression conferred target recognition to these transduced cells, a co-culture experiment was performed. The KK-LC-1-TCR transduced T cells were co-cultured with DU-145, DU-145+A*01, MKN-45, and MKN-45+A*01. After 18 hours, the supernatant was collected, and an ELISA was performed on the supernatant for Interferon-gamma (IFNγ). An increase in IFNγ release can be seen in both A*01 transduced cells, DU-145+A*01 (FIG. 6A) and MKN-145+A*01 (FIG. 6B). Furthermore, female NSG mice were injected with MKN-45 (FIG. 6C) and MKN-45-A*01 (FIG. 6D) tumors on each flank subcutaneously. The tumors were allowed to develop until they were approximately 50-100 mm³. Five NSG mice were dosed with 10×10⁶ untransduced T cells (black circles, n=6), the other 5 mice were dosed with 10×10⁶ 820 KK-LC1-TCR transduced T cells (blue triangles, n=6) via tail vein on day 1. Measurements were taken with calipers every 3-4 days. Tumor volumes are normalized to Day 1 (T₀) for each tumor (FIG. 6A-6D).

This data shows that the introduction of A*01 expression into tumors that are KK-LC-1 positive but HLA-A*01 negative leads to T cell recognition of these tumor cells. If this is broadly applicable, this would allow for previous HLA-restricted TCRs to be used in combination with a tumor-targeting virus carrying the HLA-A*01 expression vector. This would not only limit cross-reactivity, as other patient cells would not carry the HLA-A*01 allele, but also allow for T cell therapies to be used in an HLA-independent manner.

### Example 3: A*02 Expression in NY-ESO-1+/A*02-Tumor Lines Confers Recognition by NY-ESO-1-TCR Transduced T Cells

The NY-ESO-1-TCR has been shown to be very effective at recognizing NY-ESO-1 positive tumors that naturally express the HLA-A*02 protein (A*02). The limitations of any TCR therapy are 2-fold: (1) expression of the target peptide, here NY-ESO-1, and (2) expression of the correct HLA-A molecule that the TCR and peptide bind. Whether expression of the HLA molecule was sufficient to confer recognition in NY-ESO-1 positive but A*02 negative cell lines is an open question. The purpose of these experiments was to evaluate whether NY-ESO-1-TCR transduced T cells would recognize tumors that express NY-ESO-1 but were not naturally A*02 positive after transduction with an A*02 expression vector.

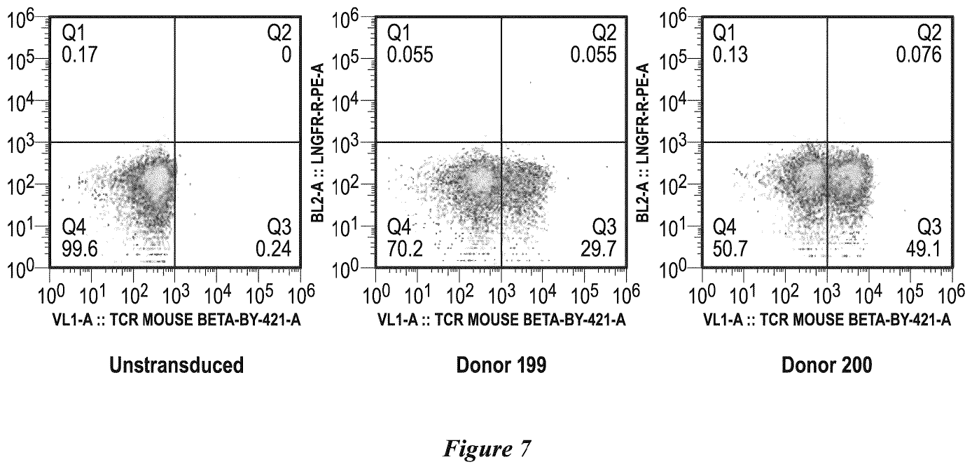

T cells from 2 healthy donors were transduced with the NY-ESO-1-TCR retrovirus. After 4 days, the cells were stained with an antibody that binds the mouse T-cell receptor beta constant region. This region is only present on the NY-ESO-1-TCR and will not be detected on untransduced cells. T cells show transduction rates of approximately 40% across all donors (FIG. 7).

Target cells were made by transducing two NY-ESO-1 positive lines, MEL-624.28 and EKVX, with a pBABE retroviral construct containing a puromycin resistance cassette and an HLA-A*02 encoding element MEL-624.28 naturally express HLA-A*03, and EKVX naturally express HLA-A*1. The cells were transduced with the pBABE construct and then treated with the appropriate amount of puromycin for 10 days to select for a puromycin resistant population which are NY-ESO-1 positive and A*02 positive.

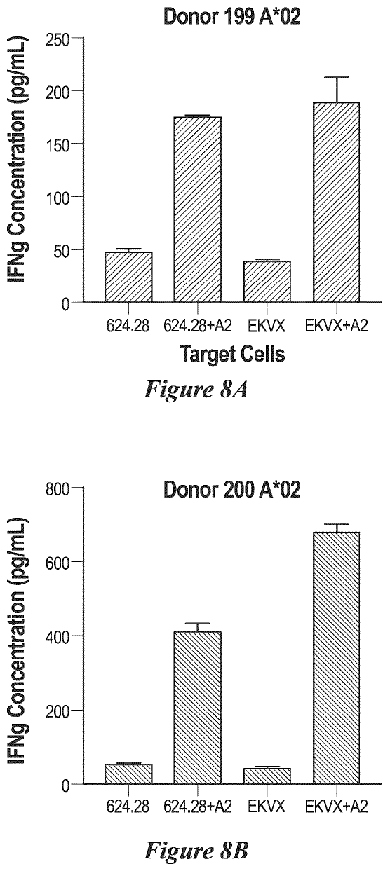

To test whether A*02 expression conferred target recognition to these transduced cells, a co-culture experiment was performed. The NY-ESO-1-TCR transduced T cells were co-cultured with MEL-624.28, MEL-624.28+A*02, EKVX, and EKVX+A*02. After 18 hours, the supernatant was collected, and an ELISA was performed on the supernatant for Interferon-gamma (IFNγ). An increase in IFNγ release can be seen in both A*02 transduced cells across 2 donors, 199 (FIG. 8A) and 200 (FIG. 8B).

This data shows that the introduction of A*02 expression into tumors that are NY-ESO-1 positive but HLA-A*02 negative leads to T cell recognition of these tumor cells. If this is broadly applicable, this would allow for previous HLA-restricted TCRs to be used in combination with a tumor-targeting virus carrying the HLA-A*02 expression vector. This would not only limit cross-reactivity, as other patient cells would not carry the HLA-A*02 allele, but also allow for T cell therapies to be used in an HLA-independent manner.

### Example 4: An Oncolytic Virus Encoding an IHITT Cassette Allows Non-HLA-A*0101 Tumor Cells to be Susceptible to 820 TCR-T Killings In Vitro and Vivo

HLA-A*0101 is expressed in about 15% US populations, leaving 85% of patients possessing KK-LC-1⁺ tumors ineligible for KK-LC-1/HLA-A1 specific 820 TCR-T treatment. The proposed oncolytic virus approach could eliminate HLA matching for 820 TCR-T treatment, significantly expanding patient coverage.

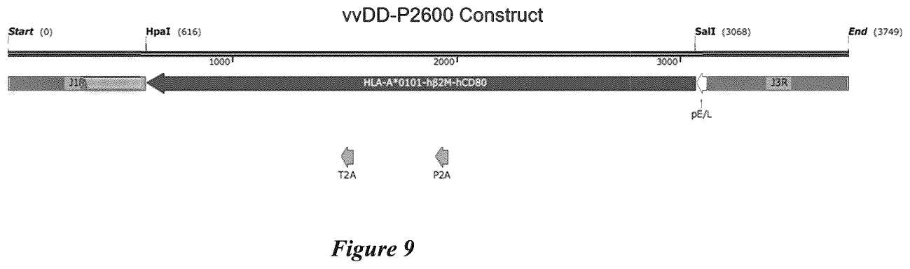

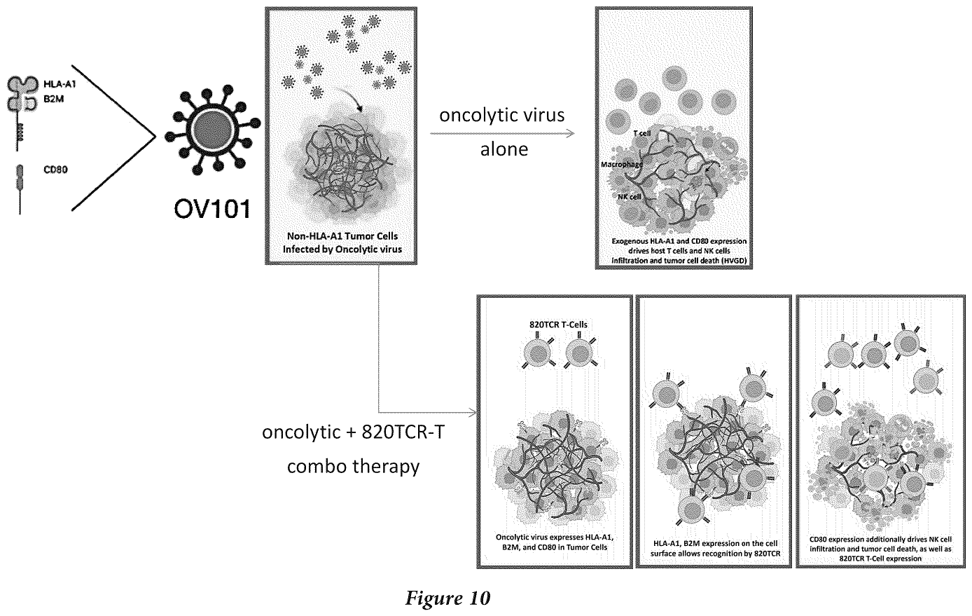

A cassette encoding the genes for β-2 microglobulin, the immune costimulatory molecule CD80, and the HLA-A*0101 proteins was constructed by common molecular cloning techniques. This cassette was inserted into the TK region of a WT vvDD vaccinia strain using homologous recombination of the flanking sequences and complete insertion was confirmed via Sanger sequencing. (FIG. 9). This virus, vvDD-P2600, was propagated using standard growth techniques for vaccinia.

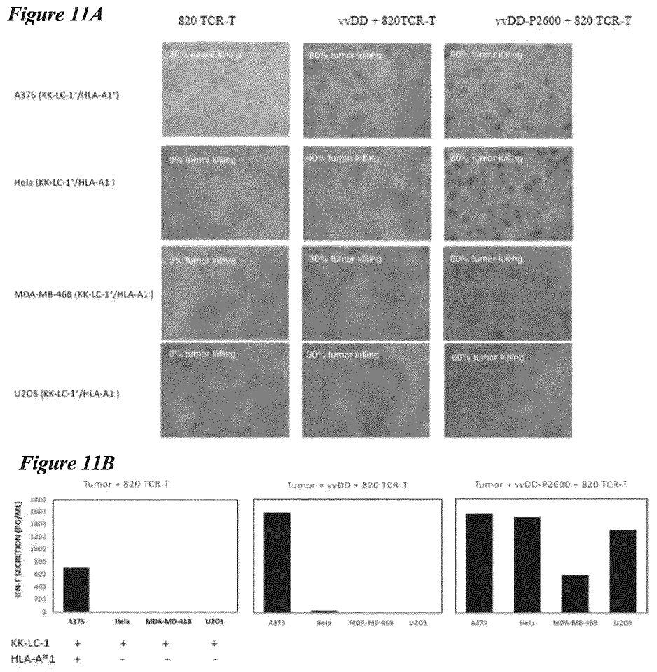

Tumor cells that express KK-LC-1 but are not genetically HLA-A*0101 were infected with vvDD and vvDD-P2600 at different MOIs. Infection was confirmed with western blots against the constituent components of the B2M-HLA-CD80 cassette. After establishing an infectious dose that was not cytotoxic, infected cells were placed in co-culture with T-Cells that express the anti-KK-LC-1-TCR. Cell killing was observed microscopically and supernatants harvested from the co-cultures showed specific IFNγ production from the anti-KK-LC-1-TCR T-Cells only when they were co-cultured with cells co-infected with the vvDD-P2600 virus (FIG. 11A).

Tumor cells that are not genetically HLA-A*0101 were infected with vvDD and vvDD-P2600 at different MOIs. Infection was confirmed with Western blots against the constituent components of the B2M-HLA-CD80 cassette (FIG. 11B).

Tumor cells that have acquired a mismatched or allogeneic HLA but lack tumor antigen expression may escape TCR-T mediated killings. However, these tumor cells expressing mismatched or allogeneic HLA but having antigen loss may effectively become targets of host versus tumor effects in longer terms due to expression of allogeneic HLA.

### Example 5: An Oncolytic Virus Encoding an IHITT Cassette Allows Non-HLA-A*0201 Tumor Cells to be Susceptible to 806 NY-ESO-1 TCR-T Killings In Vitro and Vivo

As the FDA-approved Afami-cel and most on-going TCR-T trials are targeting HLA-A*0201, the most dominant haplotype accounting for about 40% North American population, it is natural to investigate vaccinia virus encoding HLA-A*0201, B2M, and CD80 (vvDD-P2700) may render non-HLA-A*0201 tumors suspectable by HLA-A*0201-specific TCR-T. Accordingly, a 806 TCR targeting the HLA-A*0201 specific NY-ESO-1/LAGE-1a:157-165 epitope will be used as effectors to pair with vvDD-P2700.

Using a similar strategy as the vvDD-P2600 encoding HLA-A*0101 was developed, HLA-A*0201 cDNA will be used for the HLA/B2M/CD80 cassette in designing the shuttle vector to construct vvDD-P2700. In an exemplary protocol, to generate new vvDD-based viruses, U2OS in one well of a 6-well plate will be infected with a parental vvDD virus at MOI of 0.5 for 1 hour, then transfected with 4 g of the shuttle vector using Lipofectamine 3000 transfection reagent following the manufacturer's recommendation. Two days later, cells will be harvested to extract viruses as previously described. The recombinant virus will be purified on U2OS cells, and its genomic integrity will be monitored by PCR and DNA sequencing for all engineered regions.

Tumor cell lines that express NY-ESO-1 and/or LAGE-1a but are not genetically HLA-A2 will be infected with vvDD-P2700 encoding the HLA-A*0201 cassette at different MOIs. Infection will be confirmed with Western blots against individual components of the HLA*0201-B2M-CD80 cassette. After establishing an infectious dose that is not cytotoxic, infected cells will be placed in coculture with the 806 NY-ESO-1 TCR-T.

Co-culture of non-HLA-A2 tumors with various levels of NY-ESO-1/LAGE-1a expression will be infected with vvDD-P2700 encoding HLA-A*0201 based on the optimal infection dose and time course obtained from studies described above.

Total PBMC will be activated with Immunocult CD3/CD28 activator in serum-free Immunocult medium (STEMCELL Technologies, Cambridge, MA). One day following activation, cells will be plated at 2E6 cells/mL in a Retronectin-coated 6-well plate for a 10-minute spinoculation at 32 degrees. Prior to adding T cells, retrovirus encoding 806 TCR at 5 MOI will be spun down onto Retronectin-coated plate for 2 hours at 32 C. The use of total T cells instead of CD8⁺ T cells alone was warranted by the high-affinity of the “all-natural” 806 TCR at 130 nM (T-Cure Patent Publication No. WO2022093333-A1). As a matter of fact, 806 TCR-transduced CD4⁺ T cells work equally well with CD8⁺ T cells in terms of killing HLA-A2+/NY-ESO-1⁺ tumors in vitro. The spinoculation and subsequent activation usually result in a 70% transduction and 100-200-fold expansion of mixed CD4⁺ and CD8⁺ T cells at the end of the 12-day culture in the presence of IL-2. Quality controls will primarily rely on tetramer or V3 staining and recognition of tumor cells in an IFN-7 release assay.

Equal numbers of 806 NY-ESO-1 TCR-T i.e. an MOI of 1 will be used based on experience in the vvDD-P2600 study. Cell killings will be observed and enumerated by counting tumor cells under the microscope; whereas supernatants will be harvested from the cocultures for IFN-7 ELISA. Red florescence-labeled tumor cells will be investigated with Incucyte for real time tumor killing effects by the otherwise non-compatible 806 TCR-T. Melanoma cell line 624.38Mel (HLA-A*0201⁺/NY-ESO-1⁺) will be used as a positive control; while HPAF-II (NY-ESO-1⁻/LAGE-1a⁻/HLA-A*0201⁻) as a negative control.

In the case that vvDD-P2700 is too cytotoxic to tumor cells, variants of the vvDD-P2700 with genetic deletions of relevant to its oncolytic activities will be developed. The strategies may reduce the cytotoxic effects of vvDD alone and demonstrate tumor killings by the effector 806 TCR-T.

### Example 6: An Oncolytic Virus Encoding an IHITT Cassette Allows Non-HLA-A*0101 Tumor Cells to be Susceptible to 820 KK-LC-1 TCR-T Killings in a TNBC Model

Oncolytic virus has the advantage of opening up the vasculature of solid tumors to allow access of T cells to the tumor microenvironment. Oncolytic virus that switches the HLA haplotype of the tumor may also induce host-versus-tumor effects with or without additional TCR-T cell therapy. The HLA/B2M/CD80 cassette encoded by vvDD may function as a vaccine to stimulate and expand infused TCR-T in the tumor microenvironment. These properties of oncolytic virus make them potentially synergistic partners with TCR-T in treating solid tumors.

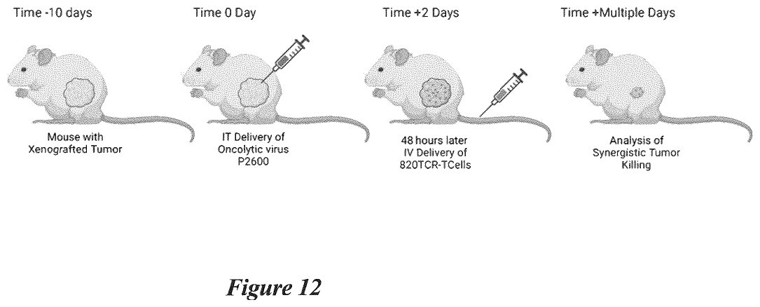

The MDA-MB-468 (KK-LC-1+/HLA-A1−) triple negative breast cancer (TNBC) line was efficiently killed by 820 KK-LC-1 TCR-T only after pre-infection with vvDD-P2600 for HLA switching (FIG. 11A). The cell line was known to form tumor mass in NSG mice and has been used in various therapeutic models36. In the exemplary study diagramed in FIG. 12, female NSG mice are injected subcutaneously in mammary fat pads with Matrigel, and monitored until tumors are palpable. Then mice will be divided into two treatment groups (FIG. 12):
i) Mice in group I are treated with control virus vvDD parental construct intratumorally, and 48 hours later, KK-LC-1 TCR-T-Cells are delivered via tail vein.
ii) Mice in group II are treated with vvDD-P2600 intratumorally, and 48 hours later, 820 KK-LC-1 TCR-T cells are delivered via tail vein injection.

Tumor growth will be monitored using a caliper twice per week. Splenocytes and tumor tissues are preserved after sacrifice for investigation of presence of 820 TCR-T and correlation with therapeutic efficacies. The therapeutic effects will be confirmed in at least two repeats of the whole experiment. At least in one study, the survival of animals will be investigated over time to compare survival benefits of the vvDD-P2600/820 TCR-T combo therapy with a vvDD-control/820 TCR-T combo therapy. [00334]vvDD strain is known for infecting human tumors through either systemic and intra-tumoral injection in mice. For simplicity, this study will be focused on the intra-tumoral approach. Effective virial infection and 820 KK-LC-1 TCR-T infiltration of the tumor sites will be investigated through IHC staining of the specific HLA-A*0101 and the specific 820 TCR V0, respectively.

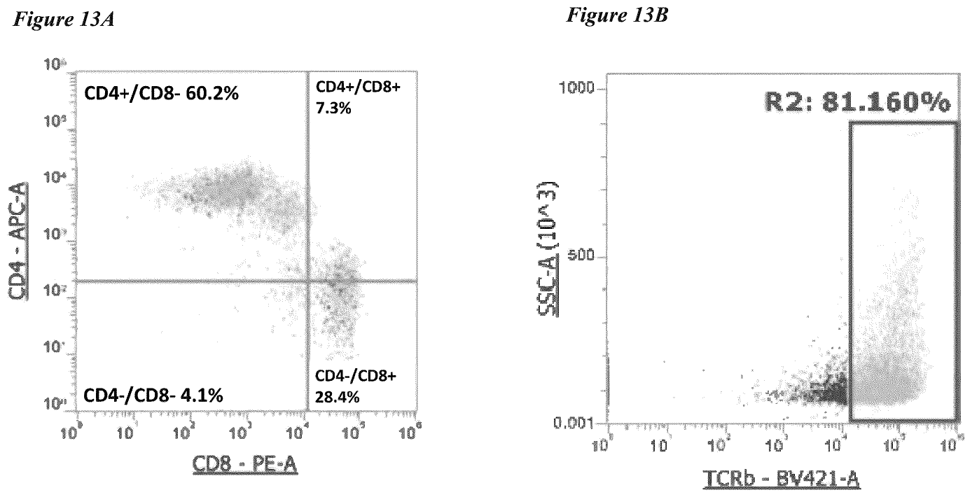

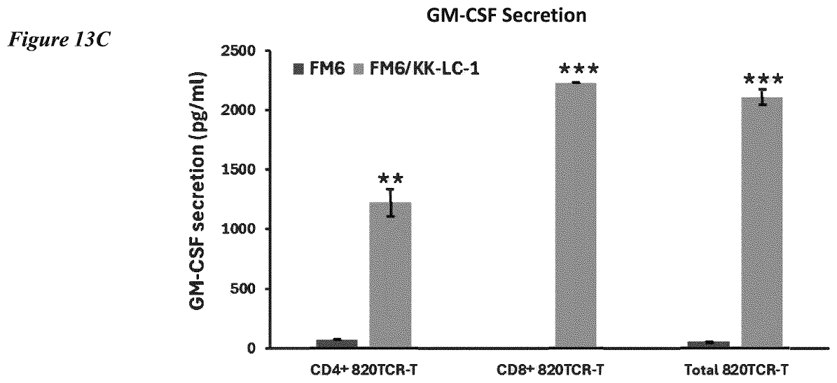

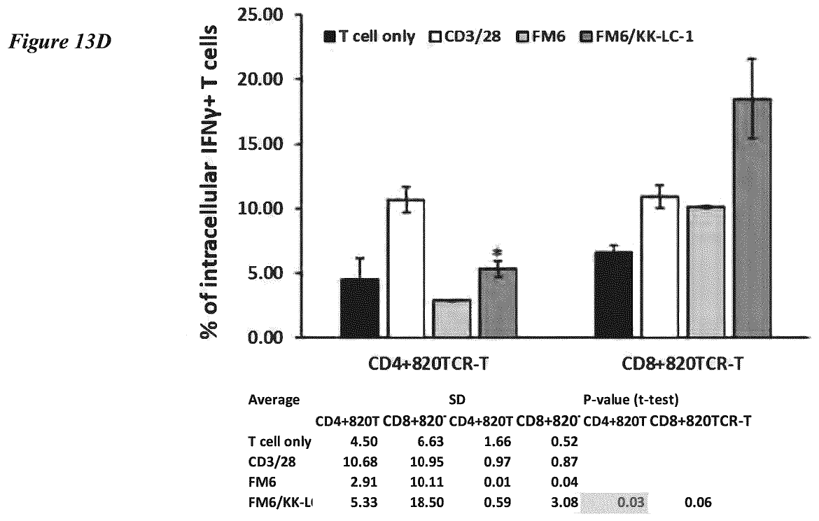

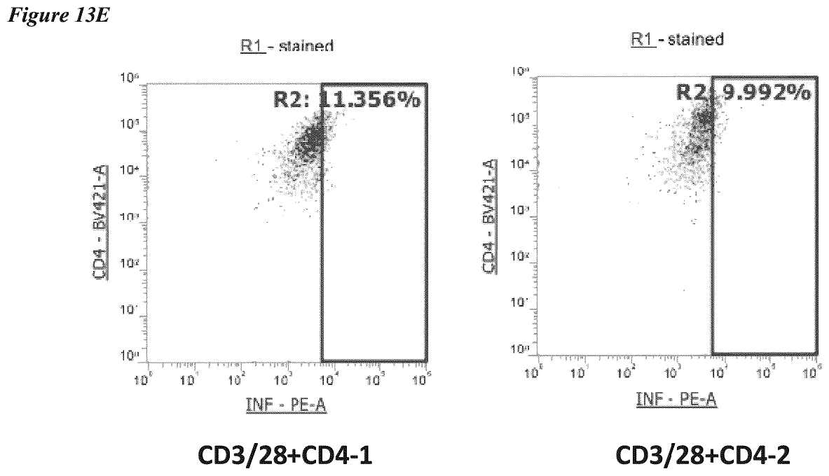

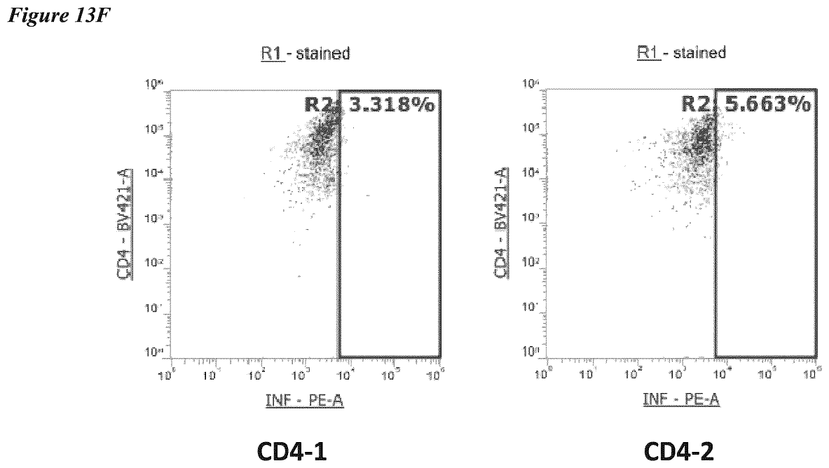

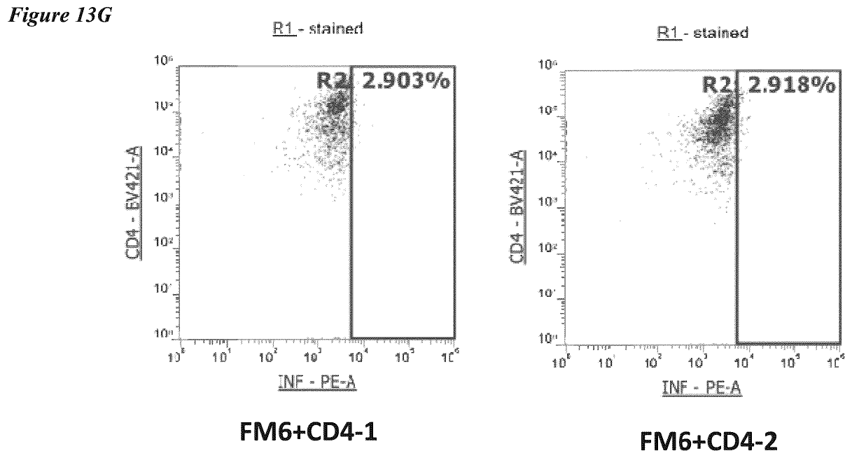

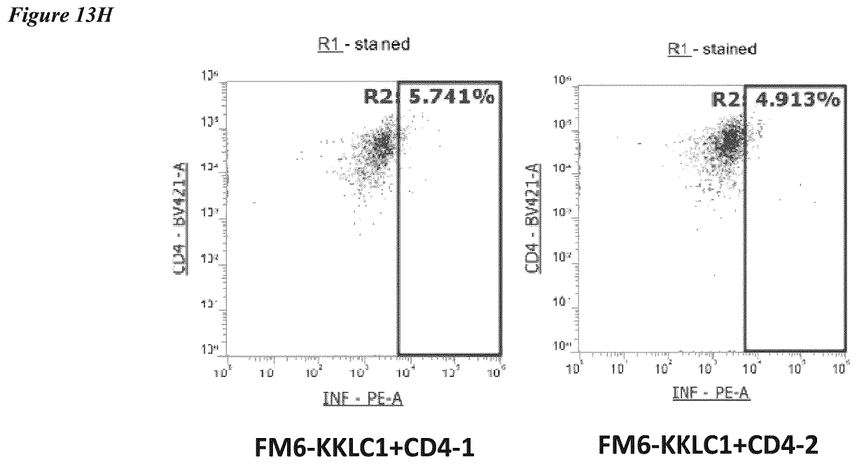

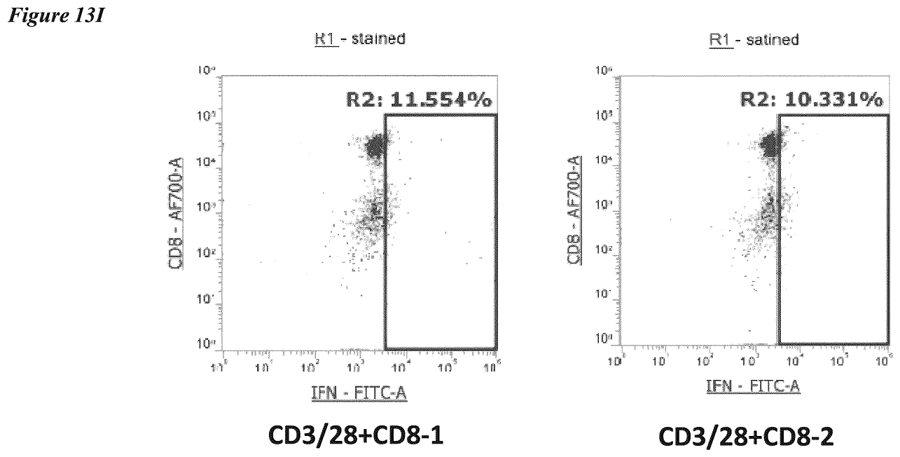

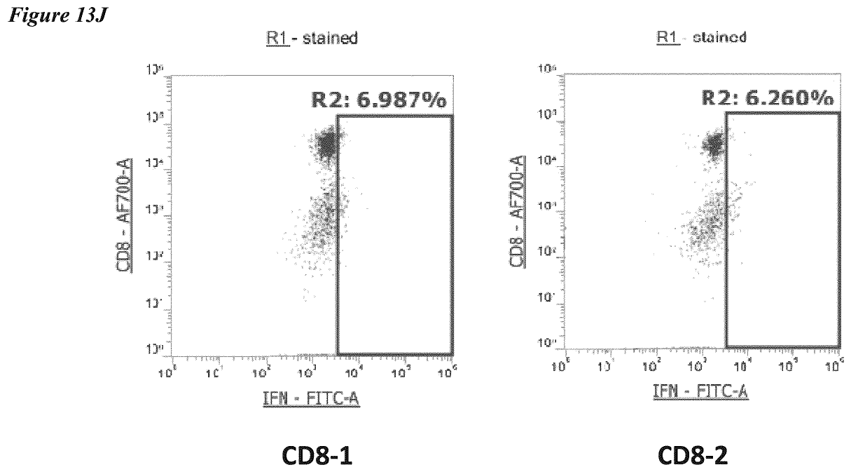

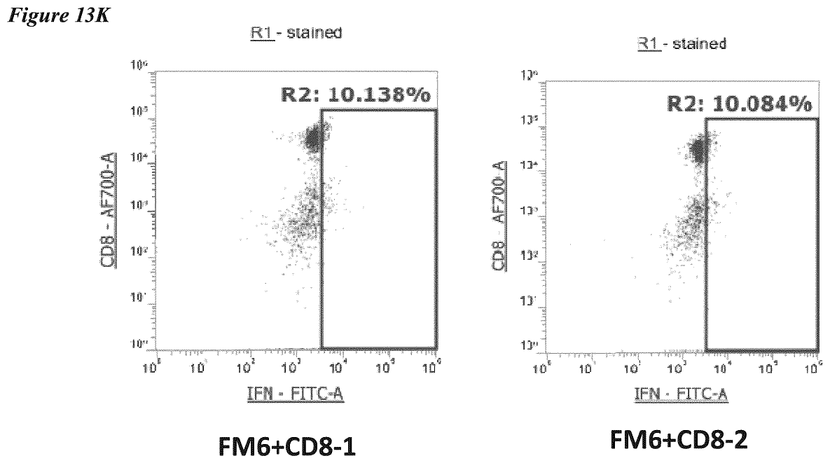

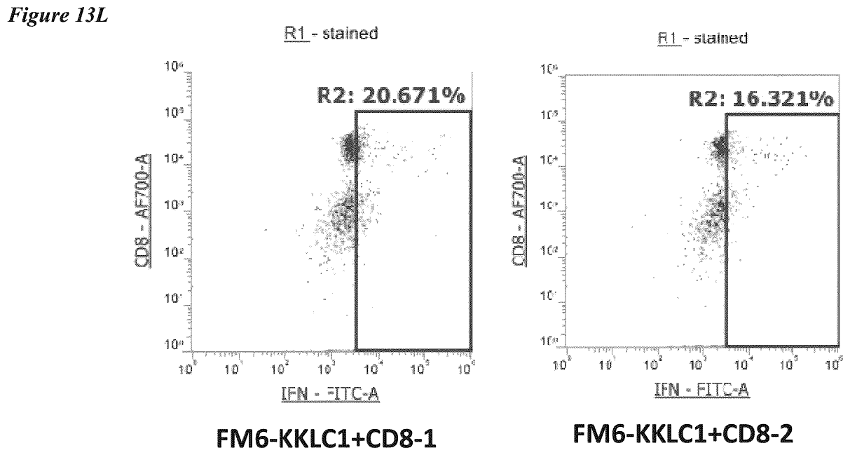

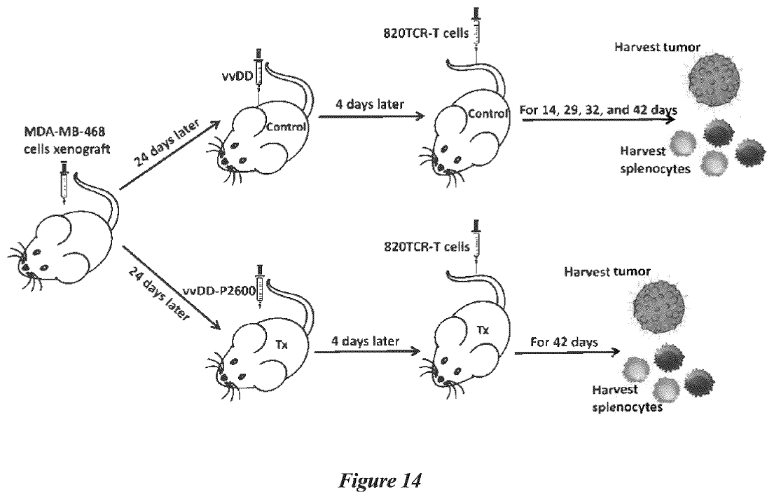

In the study use for the data described in FIG. 13, 820TCR-T cells are expanded for 14 days while monitoring their quality before use in vivo experiments such as shown in FIG. 14. In FIGS. 13A and 13B, thawed 820TCR-T cells (~4e6) were seeded in two wells (cell density 2e6 cells/well) of 6-well-plate and reactivated by CD3/28 activator (25 pl/ml) and cultured with ImmunoCult-XF T Cell Exp Medium is supplemented with rhIL-2 (300IU/ml), rhIL-15 (100 ng/ml), and β-ME. (1000×). Half of the media is changed every three days and expansion is allowed for 14 days. On Day 14, 200 μl of resuspended T cells were removed and washed with staining buffer (0.5% BSA in DPBS) in 5 ml flow tubes (1e5 cells/tube) at 500 g for 5 min, 4° C. Staining buffer is removed and cells are incubated with 200 pl staining buffer with 2 μl of each antibody for CD4 (CD4-APC), 8 (CD8-PE) and TCR (TCRβ-BV421) on ice in the dark for 30 minutes. The extra antibodies are washed with 2 ml of staining buffer and cells are spun down at 400 g for 5 min, 4° C. resuspend cells in 300 μl of staining buffer and loaded for flow cytometry for quality control (QC) analysis. FIG. 13A shows the population of CD4+/CD8+ cells, and FIG. 13B shows the percentage of 820TCR positive T cells.

Expanded 820TCR-T cells were separated into CD4+ and CD8+ populations using MASC microbeads and columns according to the manufacturer's instructions. The separated CD4+, 8+, and total 820TCR-T cells (1e5 cells/well) co-cultured with FM6 and FM/KK-LC-1 (2e4 cells/well) in the flat bottom 96-well-plate overnight and the supernatant was removed for GM-CSF ELISA assay based on the routine ELISA protocol. **p<0.01; ***p<0.001, FM6 vs FM6/KK-LC-1, t-test, n=3. FIG. 13C shows robust secretion of GM-CSF, a hematopoetic growth factor and immune modulator secreted by activated T cells, from CD4+, CD8+, and total TCR-T cells.

FM6 and FM/KK-LC-1 (2e4 cells/well) were planted in the U-bottom 96-well-plate and let them settle down for 2 hours prior to coculture (6 hours) with the separated CD4+ and 8+ 820TCR-T cells (2e5 cells/well). One hour after coculture, Brefeldin (1000×) was added to block the IFNγ secretion and incubate cells for 5 hours. Then resuspended T cells were harvested and stained with IFNγ and CD4+/8+ antibodies (CD4-BV421/IFN-PE and CD8-AF700/IFN-FITC), followed by Flow cytometry assays. FIG. 13D shows the percentage of intracellular IFNg+ T cells in CD4+ 820TCR-T cells and CD8+820 TCR-T cells. CD3/28 activators were used as the positive control and T cells only as the negative control. *p<0.05, FM6 vs FM6/KK-LC-1, t-test, n=2. Flow cytometry data used to determine the percentages shown in FIG. 13D are shown in FIG. 13E-13L.

### Example 7: Exemplary Study Design—Infection Rate and Presence of 820 KK-LC-1 TCR-T in Tumor Microenvironment and Splenocytes Over Time

This study will assess the potential of vvDD-P2600 encoding the HLA-A*0101 cassette as a vaccine for activating and expanding 820 KK-LC-1 TCR-T in vivo. The vaccine property of vvDD-P2600 makes it suitable as an alternative to chemotherapy aiming at lymphodepletion to combine with TCR-T in human patients.

In an exemplary experiment, 8 NSG female mice will be used for inoculating 1e7 tumor cells/mouse subcutaneously through the fat pads, followed by intra-tumoral injection of vvDD-P2600 and intravenous injection of 820 KK-LC-1 TCR-T with the same dose and time course as experiments described above. Two mice will be sacrificed; direct enumeration of 820 TCR-T at 1, 2, 3, and 4 weeks after injection through tail vein, will be conducted in peripheral blood, tumor microenvironment as well as the spleen. The availability of TCR-Vβ17 antibody and tetramer for staining 820 TCR-T assures the accurate enumeration, help assessing the activation and expanding capability by vvDD-P2600. The following specific endpoints will be investigated in this specific aim:
i) Infection rate (%) of tumor cells by vvDD-P2600 using IHC staining of human CD80 as a biomarker for tumor cells;
ii) Presence of 820 TCR-T infiltrating the tumor micro-environment over time by IHC staining of tumor sections with human V3;
iii) Percentage of 820 TCR-T in the splenocytes over time by tetramer staining of HLA-A*0101/KK-LC-1:52-60, NTDNNLAVY (SEQ ID NO: 11).

### Example 8: Sample Study Results—Infection Rate and Presence of 820 KK-LC-1 TCR-T in Tumor Microenvironment and Splenocytes Over Time

To make the CDX model, tumours were implanted subcutaneously in ten 6-week-old female NSG mice with MDA-MB-46B cells in mammary fat pads. After 24 days, waiting until tumours were palpable (~50 mm³), mice were treated with oncolytic virus w/o HLA-A*01:01-OV101 (blank control, vvDD; OV101, vvDD-P2600) at 5e7 PFU/50 μL intratumorally. Then 4 days later, infused 5e6 of 820TCR-T cells IV.

Tumour growth was monitored for up to 42 days or tumour size reached 2000 mm³. Preserved splenocytes and tumour tissues were obtained and analyzed after sacrifice. The overall scheme is diagrammed in FIG. 14.

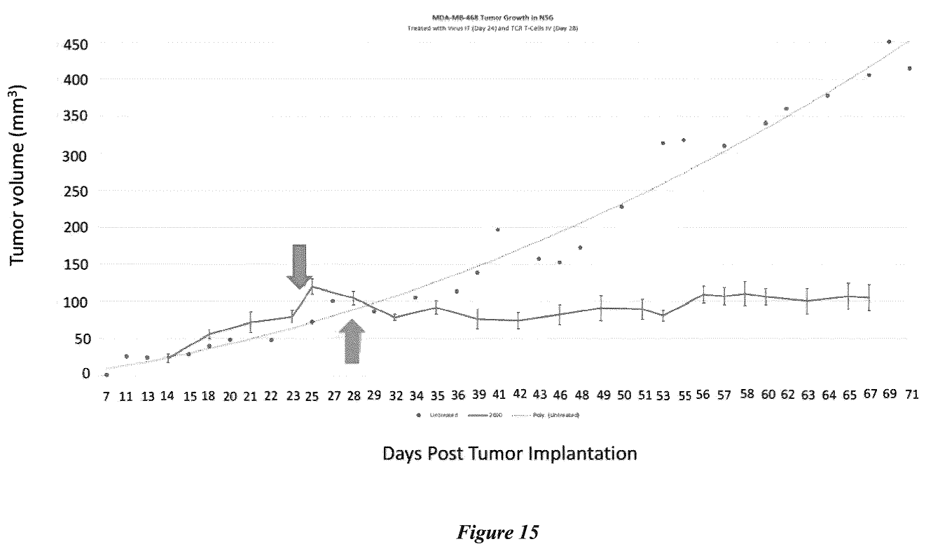

In the study that produced the data shown in FIG. 15, MDA-MB-46B tumor growth was measured in NSG mice after injection with vvDD-P2600 (OV101) on day 24 after tumor implantation and injection with expanded 820 TCR-T cells on day 28 after tumor implantation.

The data shows a marked difference in tumor volume for untreated tumors relative to treated tumors over time. Whereas tumor volume increased to about 400 mm³ after 67 days for untreated tumors, tumor volume remained about 100 mm³ for treated tumors from day 29 to day 67.

After sacrificing each mouse, fresh tumour tissue was immediately dissected and harvested, then placed in a cold fixative solution with 1x PBS. 70% ethanol-fixed tumour tissues were immersed in PBS to rehydrate them, and they were then placed into 30% sucrose to prevent ice crystal formation until they sank to the bottom of the containers. The sucrose-immersed tissues were embedded into OCT to make 10 m cryosections on a cryostat and slides were stored at 4° C. until staining. Cryosections were brought to RT and the tissue section was encircled gently with a hydrophobic barrier pen (ImmEdge pen). Slides were incubated with 5% normal donkey serum in PBS for 1 hour at RT in a humidified chamber for blocking. 250 μL of antibody mix (mouse IgG2a, κ, anti-human HLA-ABC, Biolegend, Cat. #311402, and rabbit anti-human CD80, B7-1, abcam, Cat. #AB315832 or mouse IgG2a, κ, anti-human CD3, Biotimesinc Cat. #107701, and rabbit anti-human CD8a, Biotimesinc Cat. #100301) were added per tissue section and incubate overnight at 4° C. in a humidified chamber. Then slides were washed with PBS (3×, 5 min each, light-protected). Slides were incubated using appropriate species-specific fluorophore-conjugated secondary antibody mix (FITC anti-mouse IgG2a, K, Biolegend Cat. #400207, Alexa Fluor 647 Donkey anti-rabbit IgG, Biolegend Cat. #406414) in blocking buffer (1% donkey serum in PBS, 1:200) for 1 hour at RT in a humidified chamber. After washing with PBS (3×, 5 minutes each in the dark), coverslips were mounted onto glass slides using 1-2 drops of mounting medium with DAPI. Edges were sealed with nail polish for long-term storage. Slides were let dry and stored at 4° C. in the dark until they were imaged by fluorescence microscopy (Nikon ECLIPSE Ts2R with NIS Elements software) and merging channels by FIJI Image J.

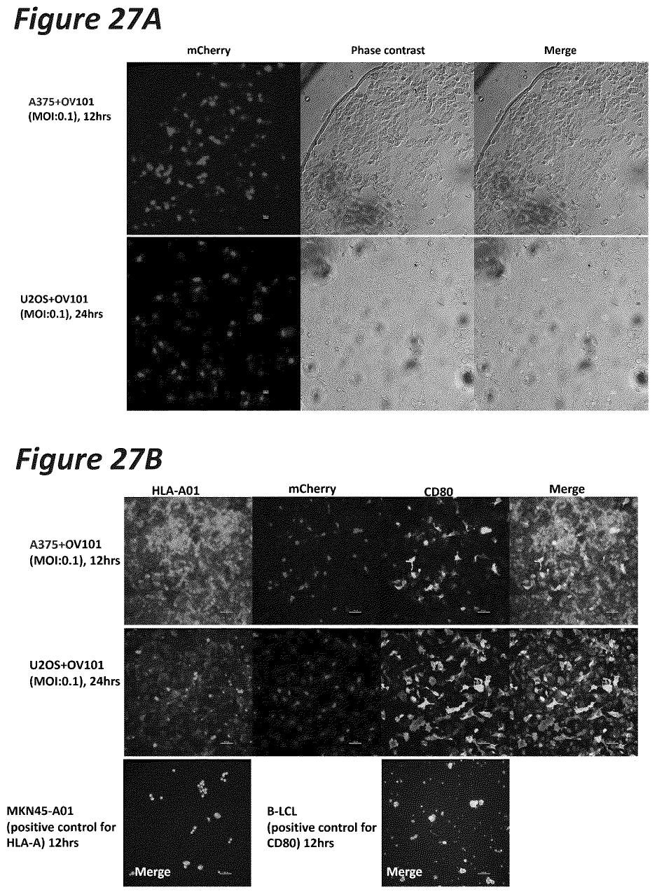

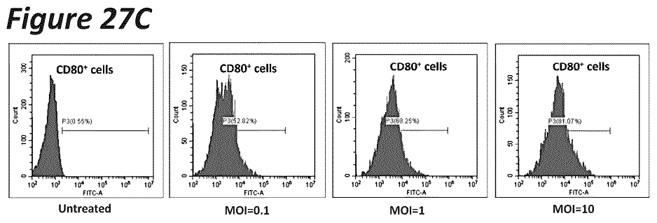

FIGS. 27A-C show the infection efficiency of vvDD-P2600-mCherry (OV101) in two unstained tumor cell lines, A375 and U20S. A375 and U20S cells were infected with OV101 at a multiplicity of infection (MOI) of 0.1 for 12 and 24 hours, respectively. Expression levels of HLA-A01 and CD80 in infected A375 and U20S cells stained with antibodies are shown in FIG. 27B, and the percentage of CD80+ A549 cells at MOI=0.1, 1, 10 are provided in FIG. 27C. Untreated cells served as the control.

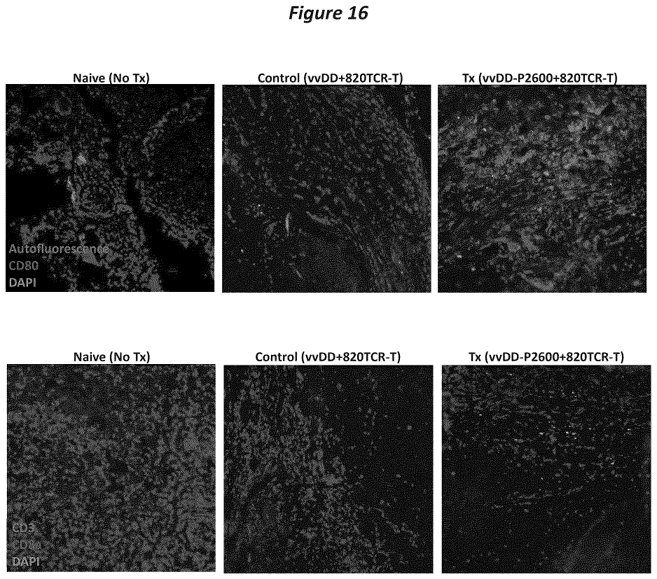

FIG. 16 shows that infection by the oncolytic virus was efficient and the infiltration of T cells in the tumour tissue post-transfection could be observed for up to 42 days.

Spleen from each mouse was obtained and a syringe butt was used to homogenize the spleen on a Petri dish. After washing with PBS to obtain a single cell suspension, the sample was passed through a mesh to remove cell debris, cells were collected and resuspended in FBS with 10% DMSO, proceeding to normal cryopreservation. After thawing the cryopreserved splenocytes, the supernatants were removed by centrifuging at 400 g, 4° C., for 5 min and the cells were resuspended with staining buffer (0.5% BSA in DPBS) in 5 ml flow tubes (5e5 cells/tube). The staining buffer was removed, and cells were incubated with antibodies of CD4 (CD4-APC), 8 (CD8-PE) and TCR (TCRβ-BV421) in the dark for 20 minutes, at 4° C. The extra antibodies were washed with the staining buffer and the cells were resuspended in 300 μl of staining buffer, and TCR-T cells were measured in the flow cytometer.

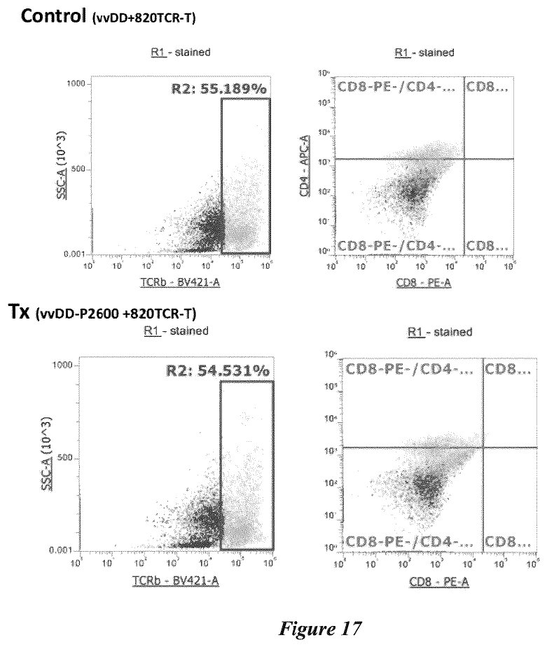

FIG. 17 shows the percentage of TCR-T cells in the isolated splenocytes of the control and treatment groups. The control cells were tested post-treatment with vvDD+820TCR-T for 32 days. The treated cells were tested post-treatment with vvDD-P2600+820TCR-T for 42 days.

### Example 9: TCR-T in Humans Using TCR-T Specific Hla Molecules Followed by Infusion of TCR-T Cells

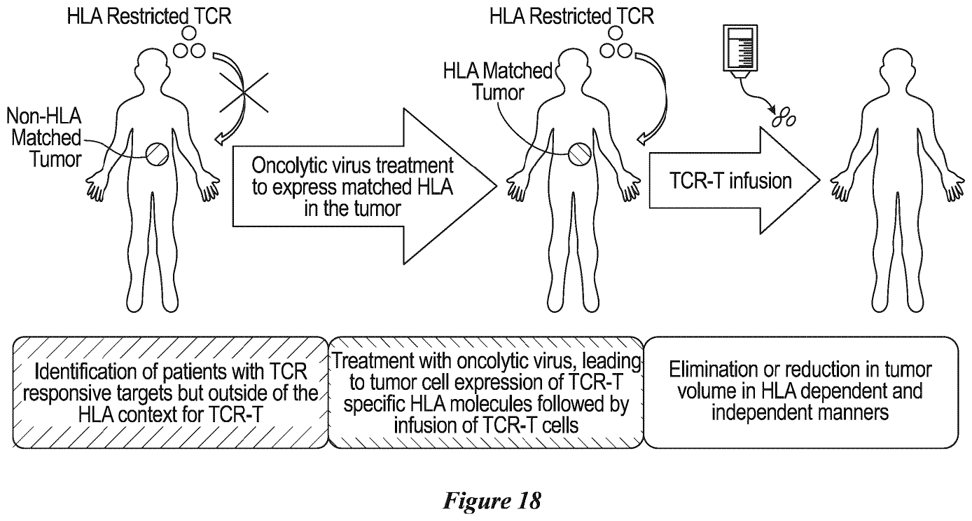

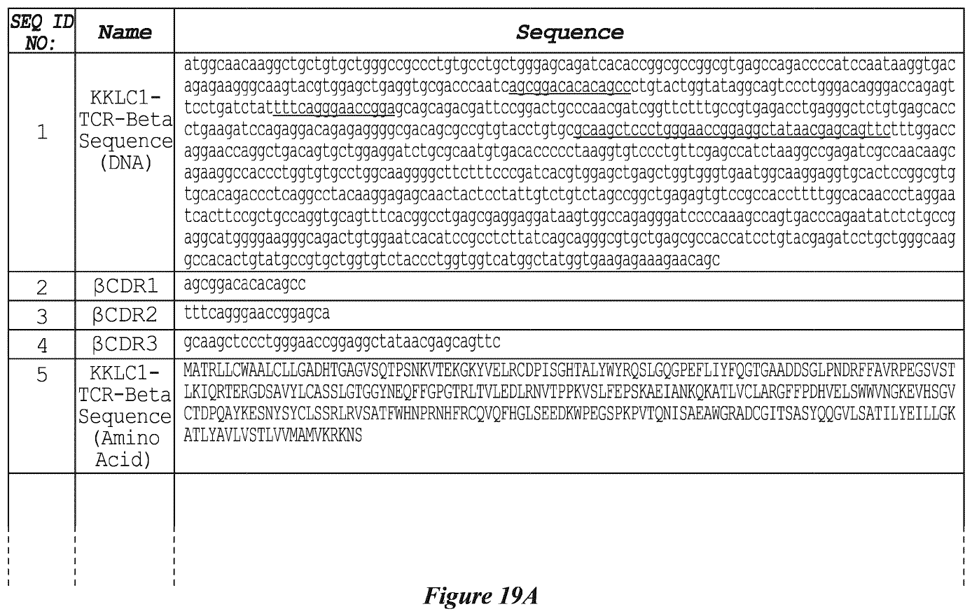

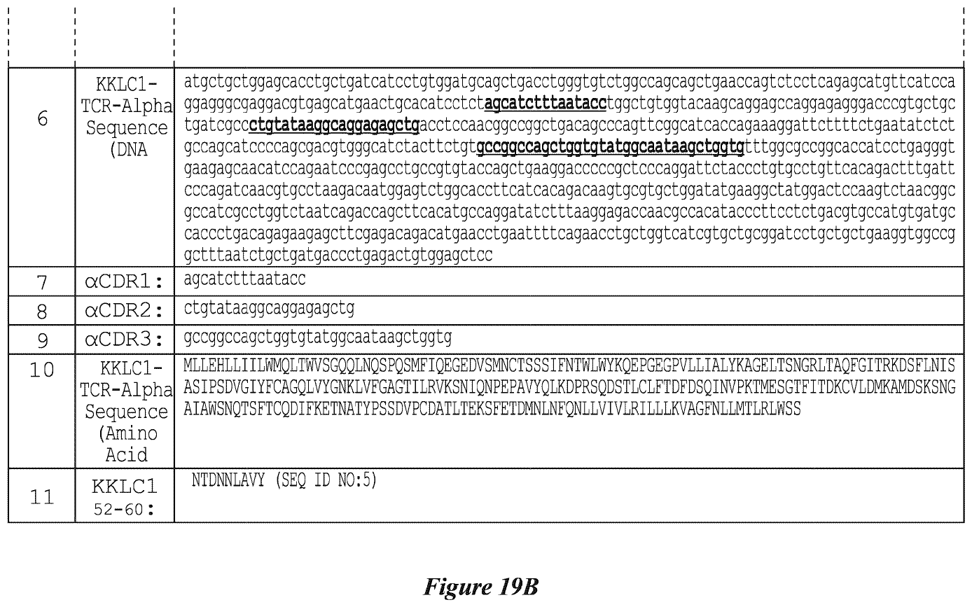

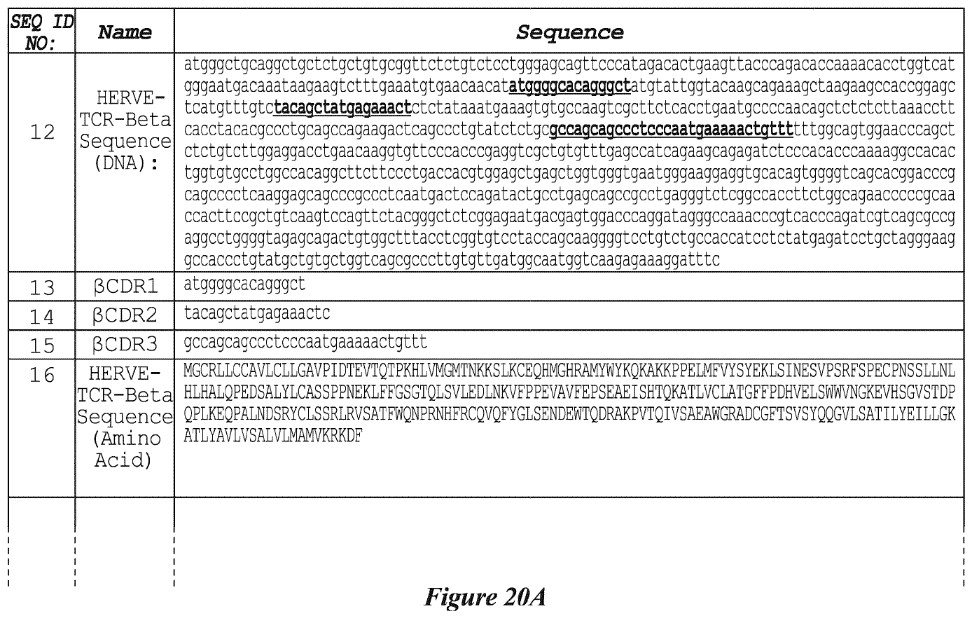

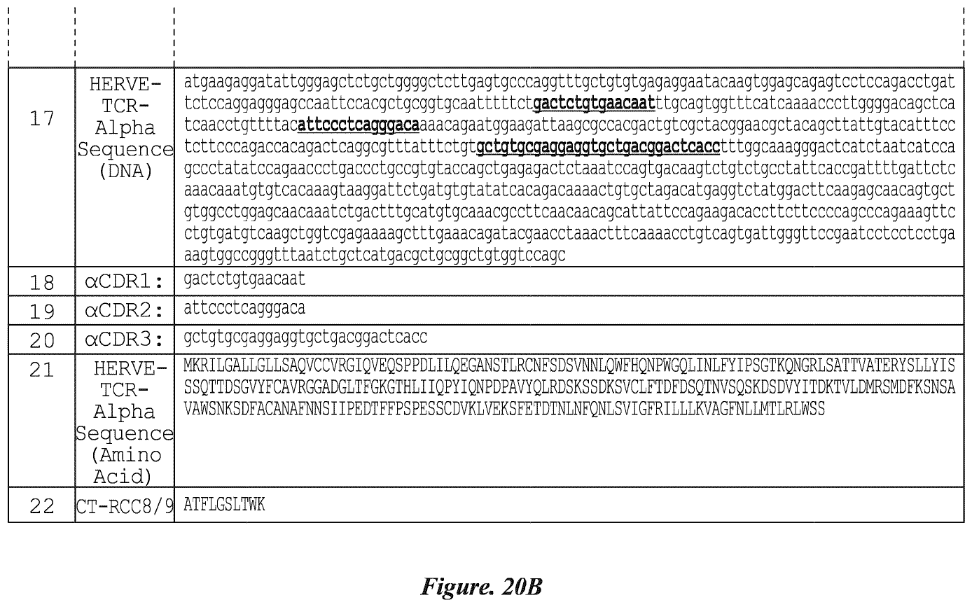

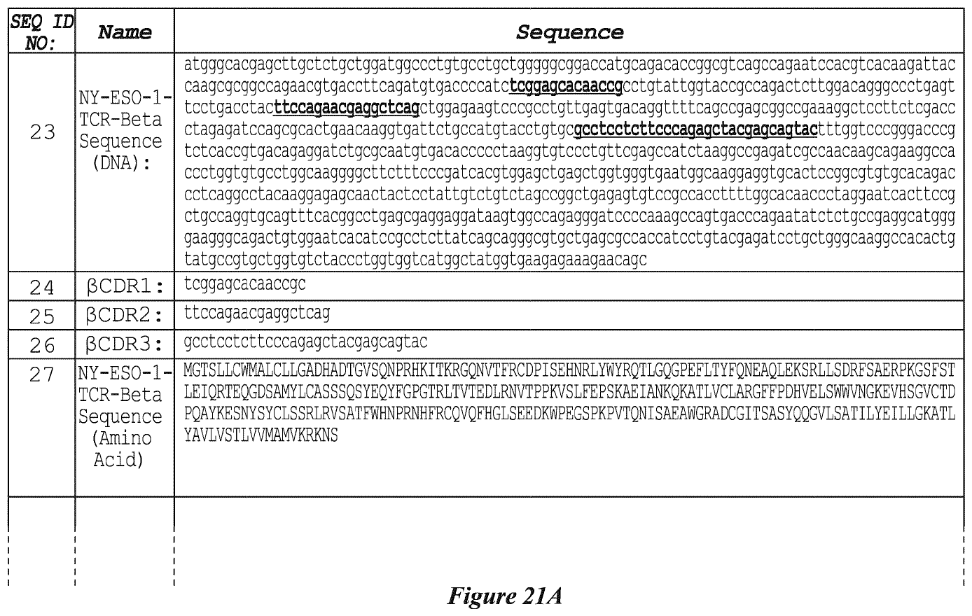

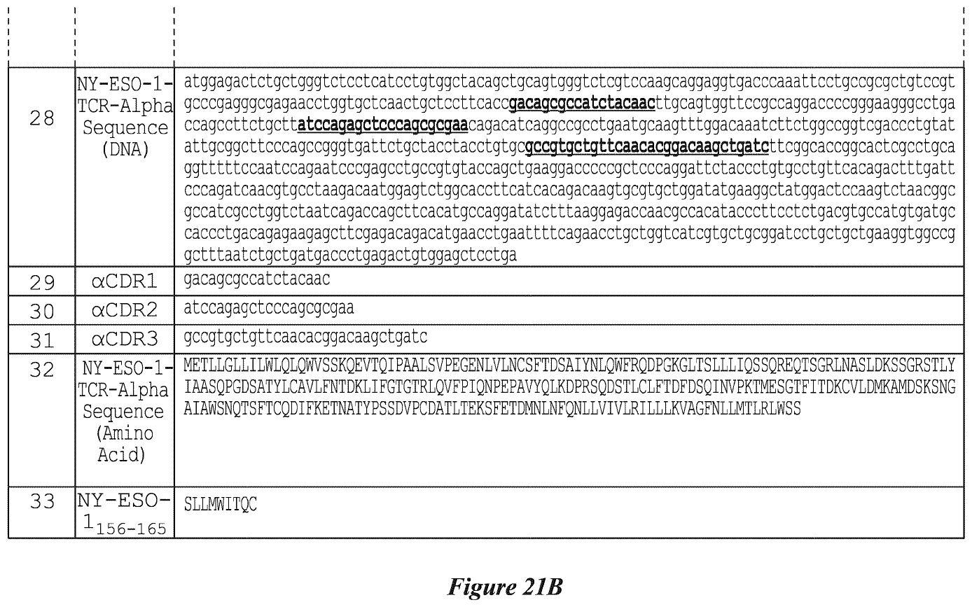

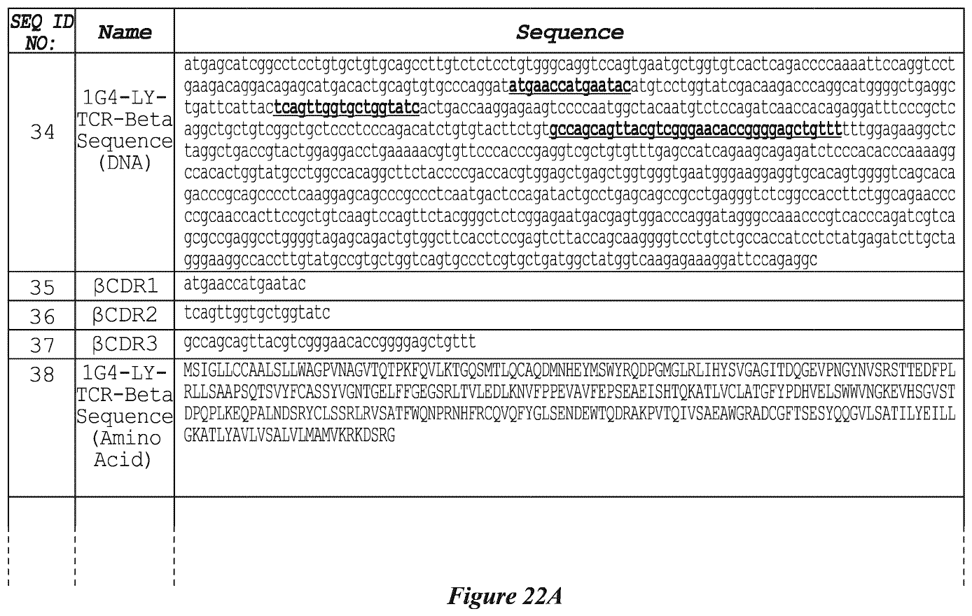

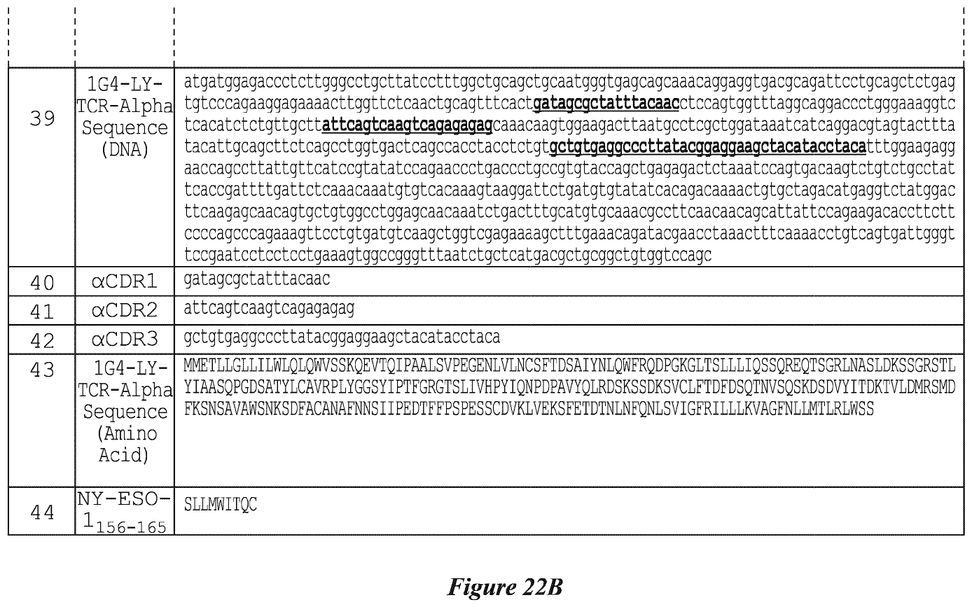

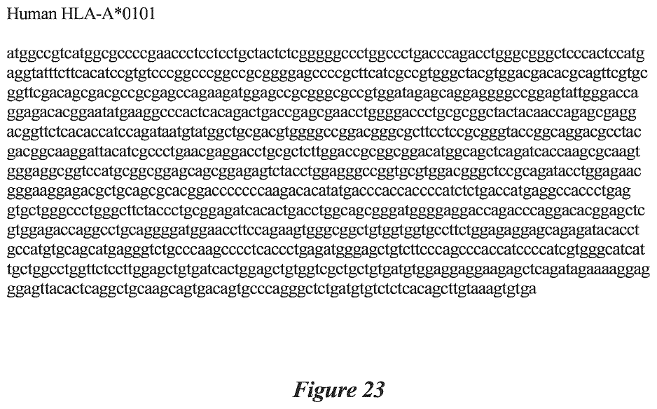

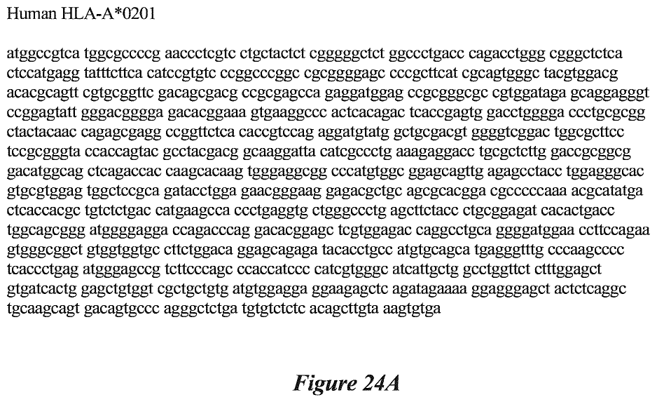

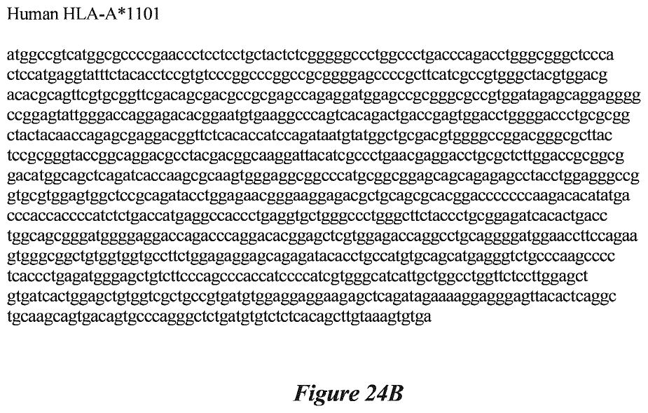

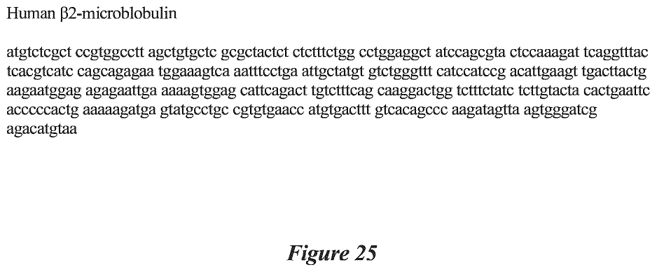

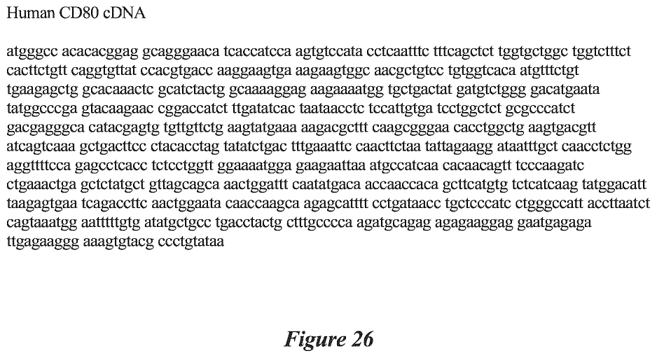

FIG. 18 shows a diagram of an exemplary treatment regimen for patients identified to have a non-HLA matched tumor and HLA restricted TCR as in the claimed invention. After treatment with an oncolytic virus that causes expression of TCR-T specific HLA molecules, tumors will be infused with TCR-T cells, and HLA dependent and HLA-independent elimination or reduction in tumor volume will be measured.

### Example 10: Phase 1 Clinical Trials of VVDD-P2600 in Combination with 820 KK-LC-1 TCR

Therapeutic efficacies of the 820 KK-LC-1 TCR-T were established in human cervical cancer and gastric cancer models using 1e7 TCR-T in a single i.v. injection in NSG mice (and FIG. 6C). The proposed combo using 5e7 PFU vvDD-P2600 encoding the HLA-A*0101 cassette followed by 5e6 820 TCR-T should result in sufficient therapeutic window to demonstrate the efficacy to MDA-MB-468 tumor line otherwise non-responsive to 820 TCR-T. Potential pitfalls are that the oncolytic effects of vvDD-P2600 may overshadow the therapeutic efficacy of 820 TCR-T. In that case, the dose of vvDD-P2600, for example, will be adjusted using one or two injections of 2e7 PFU/tumor/injection. Alternatively, a less oncolytic variant of the vvDD-P2600 can be used for injection to demonstrate more specifically the efficacy of combinational therapies.

### Example 11: Testing the Utility of Oncolytic Viruses Bearing the IHITT Cassette VVDD-P2700 with TCR-CD3 Bispecific Antibody Drugs Such as Kimmtrak for Uveal Melanoma

gp100+/HLA-A*0201− tumors such as the 624.28 melanoma line will be used as test tumor line that is not a targetable tumor by Kimmtrack. The known Kimmtrak target, the 526 melanoma line (HLA-A*0201+ and gp100+), will be a positive control while the gp100-/HLA-A*0201+ A375 melanoma line will be a negative control for in vitro experiments.

In the vitro study, neutral red labeled 624.28, 526 and A375 tumor cells will be plated on a 96-well plate at 1e4/well. Tumor cells will be infected with vvDD vector control and vvDD-P2700 (5e4 PFU/50 L), followed by addition of Kimmtrak at various amount such as 0, 10, 50, 250 pM, and same amounts of 5e4 T cells/well from a healthy donor. Tumor cell killings will be monitored by IncuCyte® in real time to show the non-targetable 624.28 tumors are now successfully killed by Kimmtrak in the presence of human PBMC.

In the animal study, 10 female NSG mice will be implanted with the 624.28 tumor cells subcutaneously (5e6 tumor cells/mouse). About 5-7 Days after the implant, 5 mice will be treated with control vvDD at 5E7 PFU/50 μL and another 5 mice will be treated with vvDD-P2700 at 5E7 PFU/50 μL. About two days later, each mouse will be injected with 1e7 human T cells and Kimmtrak at 100 pM via tail vein followed by tumor size monitoring with a caliber. The presence of vvDD control can help reduce tumor sizes, but vvDD-P2700 is expected to render Kimmtrak achieve significant tumor regression in the otherwise non-targetable 624.28 tumor model.

### References for Examples

1. Boudousquie C, Bossi G, Hurst J M, Rygiel K A, Jakobsen B K, and Hassan N J. 2017. Polyfunctional response by ImmTAC (IMCgp100) redirected CD8+ and CD4+ T cells. Immunology 152: 425-438.
2. Cole D J, Weil D P, Shamamian P, Rivoltini L, Kawakami Y, Topalian S, Jennings C, Eliyahu S, Rosenberg S A, Nishimura M I. 1994. Identification of MART-1-specific T-cell receptors: T cells utilizing distinct T-cell receptor variable and joining regions recognize the same tumor epitope. Cancer Res 54: 5265-8.
3. Clay T M, Custer M C, Sachs J, Hwu P, Rosenberg S A, Nishimura M I. 1999. Efficient transfer of a tumor antigen-reactive TCR to human peripheral blood lymphocytes confers anti-tumor reactivity. J Immunol 163: 507-13.
4. Dudley M E, Wunderlich J, Nishimura M I, Yu D, Yang J C, Topalian S L, Schwartzentruber D J, Hwu P, Marincola F M, Sherry R, Leitman S F, Rosenberg S A. 2001. Adoptive transfer of cloned melanoma-reactive T lymphocytes for the treatment of patients with metastatic melanoma. J Immunother 24: 363-73.
5. Dudley M E, Wunderlich J R, Robbins P F, Yang J C, Hwu P, Schwartzentruber D J, Topalian S L, Sherry R, Restifo N P, Hubicki A M, Robinson M R, Raffeld M, Duray P, Seipp C A, Rogers-Freezer L, Morton K E, Mavroukakis S A, White D E, Rosenberg S A. 2002. Cancer regression and autoimmunity in patients after clonal repopulation with antitumor lymphocytes. Science 298: 850-4.
6. Feola, S., Chiaro, J., Martins, B. & Cerullo, V. 2020. Uncovering the Tumor Antigen Landscape: What to Know about the Discovery Process. Cancers 12: 1660.
7. Garrido F, Algarra I, Garcia-Lora A M. 2010. The escape of cancer from T lymphocytes: immunoselection of MHC class I loss variants harboring structural-irreversible “hard” lesions. Cancer Immunol Immunother 59: 1601-6.
8. Harper J, Adams K J, Bossi G, Wright D E, Stacey A R, Bedke N, Martinez-Hague R, Blat D, Humbert L, Buchanan H, LeProvost G S, Donnellan Z, Carreira R J, Paston S J, Weigand L U, Canestraro M, Sanderson J P, Gordon-Smith S B, Lowe K L, Rygiel K A, Powlesland A S, Vuidepot A, Hassan N J, Cameron B J, Jakobsen B K, and Dukes J. 2018. An approved in vitro approach to preclinical safety and efficacy evaluation of engineered T cell receptor anti-CD3 bispecific (ImmTAC) molecules. PLoS One 13:e0205491.
9. Hu, Z., Ott, P. A. & Wu, C. J. 2018. Towards personalized, tumour-specific, therapeutic vaccines for cancer. Nat. Rev. Immunol. 18: 168-182.
10. Hunder N N, Wallen H, Cao J, Hendricks D W, Reilly J Z, Rodmyre R, Jungbluth A, Gnjatic S, Thompson J A, Yee C. 2008. Treatment of metastatic melanoma with autologous CD4⁺ T cells against N Y-ESO-1. N Engl J Med 358: 2698-703.
11. Li R, Shah P H, Stewart T F, Nam J K, Bivalacqua T J, Lamm D L, Uchio E M, Geynisman D M, Jacob J M, Meeks J J, Dickstein R, Pearce S M, Kang S H, Jung S I, Kamat A M, Burke J M,
Keegan K A, Steinberg G D. 2024. Oncolytic adenoviral therapy plus pembrolizumab in BCG-unresponsive non-muscle-invasive bladder cancer: the phase 2 CORE-001 trial. Nat. Med. 30: 2216-2223.
12. Lun X, Ruan Y, Jayanthan A, Liu D J, Singh A, Trippett T, Bell J, Forsyth P, Johnston R N, Narendran A. 2013. Double-deleted vaccinia virus in virotherapy for refractory and metastatic pediatric solid tumors. Mol. Oncol. 7: 944-954.
13. Morgan R A, Dudley M E, Wunderlich J R, Hughes M S, Yang J C, Sherry R M, Royal R E, Topalian S L, Kammula U S, Restifo N P, Zheng Z, Nahvi A, de Vries C R, Rogers-Freezer L J, Mavroukakis S A, Rosenberg S A. 2006. Cancer regression in patients after transfer of genetically engineered lymphocytes. Science 314: 126-9.
14. Nathan P, Hassel J C, Rutkowski P, Baurain J, Butler M O, Schlaak M, Sullivan R J, et al. 2021. Overall Survival Benefit with Tebentafusp in Metastatic Uveal Melanoma. N. Engl. J. Med. 385: 1196-1206.
15. Nishihori T, Hoffman J E, Huff A, Kapoor G S, Eleftheriadou I, Zajic S, Urbano A, Suchindran S, Chisamore M, D'Souza J W, Faitg T, Rapoport A P. 2022. Safety and efficacy of letetresgene autoleucel alone or with pembrolizumab for relapsed/refractory multiple myeloma. Blood Adv. 7: 1168-1177.
16. Rapoport A P, Stadtmauer E A, Binder-Scholl G K, Goloubeva O, Vogl D T, Lacey S F, Badros A Z, Garfall A, Weiss B, Finklestein J, Kulikovskaya I, Sinha S K, Kronsberg S, Gupta M, Bond S, Melchiori L, Brewer J E, Bennett A D, Gerry A B, Pumphrey N J, Williams D, Tayton-Martin H K, Ribeiro L, Holdich T, Yanovich S, Hardy N, Yared J, Kerr N, Philip S, Westphal S, Siegel D L, Levine B L, Jakobsen B K, Kalos M, June C H. 2015. N Y-ESO-1-specific TCR-engineered T cells mediate sustained antigen-specific antitumor effects in myeloma. Nat Med. 21: 914-921.
17. Rivoltini L, Loftus D J, Squarcina P, Castelli C, Rini F, Arienti F, Belli F, Marincola F M, Geisler C, Borsatti A, Appella E, Parmiani G. 1998. Recognition of melanoma-derived antigens by CTL: possible mechanisms involved in down-regulating anti-tumor T-cell reactivity. Crit Rev Immunol 18: 55-63.
18. Robbins P F, Morgan R A, Feldman S A, Yang J C, Sherry R M, Dudley M E, Wunderlich J R, Nahvi A V, Helman L J, Mackall C L, Kammula U S, Hughes M S, Restifo N P, Raffeld M, Lee C C, Levy C L, Li Y F, El-Gamil M, Schwarz S L, Laurencot C, Rosenberg S A. 2011. Tumor Regression in Patients With Metastatic Synovial Cell Sarcoma and Melanoma Using Genetically Engineered Lymphocytes Reactive With N Y-ESO-1. J Clin Oncol 29: 917-24.
19. Robbins P F, Kassim S H, Tran T L, Crystal J S, Morgan R A, Feldman S A, Yang J C, Dudley M E, Wunderlich J R, Sherry R M, Kammula U S, Hughes M S, Restifo N P, Raffeld M, Lee C C, Li Y F, El-Gamil M, Rosenberg S A. 2015. A pilot trial using lymphocytes genetically engineered with an N Y-ESO-1-reactive T-cell receptor: long-term follow-up and correlates with response. Clin Cancer Res 21: 1019-27.
20. Rosenberg S A. 2001. Progress in human tumour immunology and immunotherapy. Nature 411: 380-4.
21. Rosenberg S A. 2014. Decade in review-cancer immunotherapy: Entering the mainstream of cancer treatment. Nat Rev Clin Oncol 11: 630-2.
22. Sanderson J P et al. 2020. Preclinical evaluation of an affinity-enhanced MAGE-A4-specific T-cell receptor for adoptive T-cell therapy. Oncoimmunology 9: 1682381.
23. Soliman H. et al. 2023. Oncolytic T-VEC virotherapy plus neoadjuvant chemotherapy in nonmetastatic triple-negative breast cancer: a phase 2 trial. Nat. Med. 29: 450-457.
24. Vackova J, Polakova I, Johari S D, Smahel M. 2021. CD80 Expression on Tumor Cells Alters Tumor Microenvironment and Efficacy of Cancer Immunotherapy by CTLA-4 Blockade. Cancers 13:1935.
25. van Amerongen R A, Tuit S, Wouters A K, van de Meent M, Siekman S L, Meeuwsen M H, Wachsmann T L A, Remst D F G, Hagedoorn R S, van der Steen D M, de Ru A H, Verdegaal E M E, van Veelen P A, Falkenburg J H F, Heemskerk M H M. 2023. PRAME and CTCFL-reactive TCRs for the treatment of ovarian cancer. Front. Immunol. 14: 1121973.

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

1. A method for increasing/upregulating the sensitivity of a population of tumor cells to a TCR-engineered T cell (TCR-T) therapy, the method comprising genetically modifying the population of tumor cells to express a tumor haplotype different from the tumor haplotype endogenous to the population of tumor cells.

2.-5. (canceled)

6. The method of claim 1, wherein expressing the tumor haplotype that is different from the tumor haplotype that is endogenous to the population of tumor cells allows for targeting the population of tumor cells with the TCR-T.

7. A method for increasing the sensitivity of a tumor cell to a TCR-engineered T cell (TCR-T) therapy comprising:
a) determining the tumor haplotype of the population of tumor cells;
b) contacting the population of tumor cells with a nucleic acid encoding a tumor haplotype different from the tumor haplotype endogenous to the tumor cells, wherein the tumor haplotype different from the tumor haplotype endogenous to the tumor cells is expressed, and wherein the population of tumor cells exhibit increased sensitivity to a TCR-T therapy.

8.-10. (canceled)

11. The method of claim 7, wherein the method comprises contacting the population of tumor cell with a nucleic acid encoding the tumor haplotype that is different from the tumor haplotype endogenous to the population of tumor cells.

12.-21. (canceled)

22. The method of claim 1, wherein the vector is a non-viral vector or viral vector.

23. The method of claim 1, wherein the vector is administered to a subject in need thereof systemically, intratumorally, and/or intravenously.

24. The method of claim 1, wherein the vector is viral vector, wherein the viral vector is selected from the group consisting of a vaccinia (pox) virus vector, herpes simplex virus vector, myxoma virus, coxsackie virus vector, poliovirus vector, Newcastle disease virus vector, retrovirus vector (including lentivirus vector or a pseudotyped vector), an adenovirus vector, an adeno-associated virus vector, a simian virus vector, a sendai virus vector, measles virus vector, foam virus vector, alphavirus vector, and vesicular stomatitis virus vector.

25.-30. (canceled)

31. The method of claim 1, wherein the tumor haplotype different from the tumor haplotype endogenous to the population of tumor cells is a HLA, NY-ESO, HERV, LAGE, MAGE, MUC, BAGE, RAGE, CAGE, SSX, PRAME, PSMA, XAGE, tyrosinase, melan-A, mutated kRAS, mutated P53, or HPV E5 and E6 tumor haplotype.

32.-37. (canceled)

38. The method of claim 1, wherein the tumor haplotype different from the tumor haplotype endogenous to the population of tumor cells is an HLA tumor haplotype, and wherein the TCR-T comprises a restricted and/or targeted TCR, wherein the restricted and/or targeted TCR-T binds to KK-LC-1, CT83, VGGL1, PLAC-1, NY-ESO-1, HERV-E, HERV-K, LAGE-1, LAGE-la, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

39.-42. (canceled)

43. The method of claim 1, wherein the population of tumor cells are from a solid tumor.

44.-45. (canceled)

46. The method of claim 1 wherein the tumor cells are in vitro or in vivo.

47.-53. (canceled)

54. The method of claim 1, wherein the TCR-T therapy comprises a TCR having antigenic specificity for MART-1, gp100, mutated p53, mutated KRAS, HPV-E6, KK-LC-1, CT83, VGGL1, PLAC-1, NY-ESO-1, HERV-E, HERV-K, LAGE-1, LAGE-1a, P1A, MUC1, MAGE-1, MAGE-A1, MAGE-A2, MAGE-A3, MAGE-A4, MACE-A5, MAGE-A6, MAGE-A7, MAGE-A8, MAGE-A9, MAGE-A10, MAGE-A11, MAGE-A12, GAGE-1, GAGE-2, GAGE-3, GAGE-4, GAGE-5, GAGE-6, GAGE-7, GAGE-8, BAGE-1, RAGE-1, CAGE, LB33/MUM-1, NAG, MAGE-Xp2 (MAGE-B2), MAGE-Xp3 (MAGE-B3), MAGE-Xp4 (MAGE-B4), brain glycogen phosphorylase, MAGE-C1/CT7, MAGE-C2, SSX-1, SSX-2 (HOM-MEL-40), SSX-3, SSX-4, SSX-5, SCP-i, PRAME, PSMA, tyrosinase, melan-A, XAGE, mutated kRAS, mutated P53, or HPV E5 and E6.

55.-61. (canceled)

62. The method of claim 1, wherein the TCR comprises the amino acid sequences of SEQ ID NO: 5 and/or 10, SEQ ID NO: 16 and/or 21, SEQ ID NO: 16 and/or 21, or SEQ ID NO: 38 and/or 43.

63.-71. (canceled)

72. The method of claim 1, wherein the vector comprises the nucleic acid sequence of SEQ ID NO: 1 and the nucleic acid sequence of SEQ ID NO: 6, or comprises a nucleic acid encoding for the amino sequence of SEQ ID NO: 5 and a nucleic acid encoding for the amino sequence of SEQ ID NO: 10, or wherein the vector comprises the nucleic acid sequence of SEQ ID NO: 12 and the nucleic acid sequence of SEQ ID NO: 17, or comprises a nucleic acid encoding for the amino sequence of SEQ ID NO: 16 and a nucleic acid encoding for the amino sequence of SEQ ID NO: 21, or wherein the vector comprises the nucleic acid sequence of SEQ ID NO: 23 and the nucleic acid sequence of SEQ ID NO: 28, or comprises a nucleic acid encoding for the amino sequence of SEQ ID NO: 27 and a nucleic acid encoding for the amino sequence of SEQ ID NO: 32, or wherein the vector comprises the nucleic acid sequence of SEQ ID NO: 34 and the nucleic acid sequence of SEQ ID NO: 39 or comprises a nucleic acid encoding for the amino sequence of SEQ ID NO: 38 and a nucleic acid encoding for the amino sequence of SEQ ID NO: 43.

73.-75. (canceled)

76. The method of claim 1, wherein the vector comprises a nucleic acid sequence encoding the βCDR1, βCDR2, and βCDR3 of SEQ ID NO: 1 and a nucleic acid sequence encoding the αCDR1, αCDR2, and αCDR3 of SEQ ID NO: 6.

77. The method of claim 1, wherein the vector comprises a nucleic acid sequence encoding the βCDR1, βCDR2, and βCDR3 of SEQ ID NO: 12 and a nucleic acid sequence encoding the αCDR1, αCDR2, and αCDR3 of SEQ ID NO: 17.

78. The method of claim 1, wherein the vector comprises a nucleic acid sequence encoding the βCDR1, βCDR2, and βCDR3 of SEQ ID NO: 23 and a nucleic acid sequence encoding the αCDR1, αCDR2, and αCDR3 of SEQ ID NO: 28.

79. The method of claim 1, wherein the vector comprises a nucleic acid sequence encoding the βCDR1, βCDR2, and βCDR3 of SEQ ID NO: 34 and a nucleic acid sequence encoding the αCDR1, αCDR2, and αCDR3 of SEQ ID NO: 39.

80.-83. (canceled)

84. A peptide comprising the amino acid sequence NTDNNLAVY (SEQ ID NO:11), ATFLGSLTWK (SEQ ID NO:22), or SLLMWITQC (SEQ ID NO:33).

85.-87. (canceled)


SEQUENCE LISTING (49 sequences)

SEQ ID NO: 1 - DNA, 921 bp, synthetic construct
  source 1..921: mol_type = other DNA
atggcaacaaggctgctgtgctgggccgccctgtgcctgctgggagcagatcacaccggcgccggcgtgagccagaccccatccaataaggtgacagagaagggcaagtacgtggagctgaggtgcgacccaatcagcggacacacagccctgtactggtataggcagtccctgggacagggaccagagttcctgatctattttcagggaaccggagcagcagacgattccggactgcccaacgatcggttctttgccgtgagacctgagggctctgtgagcaccctgaagatccagaggacagagaggggcgacagcgccgtgtacctgtgcgcaagctccctgggaaccggaggctataacgagcagttctttggaccaggaaccaggctgacagtgctggaggatctgcgcaatgtgacaccccctaaggtgtccctgttcgagccatctaaggccgagatcgccaacaagcagaaggccaccctggtgtgcctggcaaggggcttctttcccgatcacgtggagctgagctggtgggtgaatggcaaggaggtgcactccggcgtgtgcacagaccctcaggcctacaaggagagcaactactcctattgtctgtctagccggctgagagtgtccgccaccttttggcacaaccctaggaatcacttccgctgccaggtgcagtttcacggcctgagcgaggaggataagtggccagagggatccccaaagccagtgacccagaatatctctgccgaggcatggggaagggcagactgtggaatcacatccgcctcttatcagcagggcgtgctgagcgccaccatcctgtacgagatcctgctgggcaaggccacactgtatgccgtgctggtgtctaccctggtggtcatggctatggtgaagagaaagaacagc

SEQ ID NO: 2 - DNA, 15 bp, synthetic construct
  source 1..15: mol_type = other DNA
agcggacacacagcc

SEQ ID NO: 3 - DNA, 18 bp, synthetic construct
  source 1..18: mol_type = other DNA
tttcagggaaccggagca

SEQ ID NO: 4 - DNA, 39 bp, synthetic construct
  source 1..39: mol_type = other DNA
gcaagctccctgggaaccggaggctataacgagcagttc

SEQ ID NO: 5 - protein, 307 aa, synthetic construct
MATRLLCWAALCLLGADHTGAGVSQTPSNKVTEKGKYVELRCDPISGHTALYWYRQSLGQGPEFLIYFQGTGAADDSGLPNDRFFAVRPEGSVSTLKIQRTERGDSAVYLCASSLGTGGYNEQFFGPGTRLTVLEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 6 - DNA, 798 bp, synthetic construct
  source 1..798: mol_type = other DNA
atgctgctggagcacctgctgatcatcctgtggatgcagctgacctgggtgtctggccagcagctgaaccagtctcctcagagcatgttcatccaggagggcgaggacgtgagcatgaactgcacatcctctagcatctttaatacctggctgtggtacaagcaggagccaggagagggacccgtgctgctgatcgccctgtataaggcaggagagctgacctccaacggccggctgacagcccagttcggcatcaccagaaaggattcttttctgaatatctctgccagcatccccagcgacgtgggcatctacttctgtgccggccagctggtgtatggcaataagctggtgtttggcgccggcaccatcctgagggtgaagagcaacatccagaatcccgagcctgccgtgtaccagctgaaggacccccgctcccaggattctaccctgtgcctgttcacagactttgattcccagatcaacgtgcctaagacaatggagtctggcaccttcatcacagacaagtgcgtgctggatatgaaggctatggactccaagtctaacggcgccatcgcctggtctaatcagaccagcttcacatgccaggatatctttaaggagaccaacgccacatacccttcctctgacgtgccatgtgatgccaccctgacagagaagagcttcgagacagacatgaacctgaattttcagaacctgctggtcatcgtgctgcggatcctgctgctgaaggtggccggctttaatctgctgatgaccctgagactgtggagctcc

SEQ ID NO: 7 - DNA, 15 bp, synthetic construct
  source 1..15: mol_type = other DNA
agcatctttaatacc

SEQ ID NO: 8 - DNA, 21 bp, synthetic construct
  source 1..21: mol_type = other DNA
ctgtataaggcaggagagctg

SEQ ID NO: 9 - DNA, 33 bp, synthetic construct
  source 1..33: mol_type = other DNA
gccggccagctggtgtatggcaataagctggtg

SEQ ID NO: 10 - protein, 265 aa, synthetic construct
MLLEHLLIINMQLTWVSGQQLNQSPQSMFIQEGEDVSMNCTSSSIFNTWLWYKQEPGEGPVLLIALYKAGELTSNGRLTAQFGITRIESFLNISASIPSDVGIYFCAGQLVYGNKLVFGAGTILRVKSNIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 11 - protein, 9 aa, synthetic construct
NTDNNLAVY

SEQ ID NO: 12 - DNA, 921 bp, synthetic construct
  source 1..921: mol_type = other DNA
atgggctgcaggctgctctgctgtgcggttctctgtctcctgggagcagttcccatagacactgaagttacccagacaccaaaacacctggtcatgggaatgacaaataagaagtctttgaaatgtgaacaacatatgtattggtacaagcagaaagctaagaagccaccggagcatggggcacagggcttcatgtttgtcctctataaatgaaagtgtgccaagtcgcttctcacctgaatgccccaacagctctctcttaaacctttacagctatgagaaactcacctacacgccctgcagccagaagactcagccctgtatctctgctttggcagtggaacccagctgccagcagccctcccaatgaaaaactgtttctctgtcttggaggacctgaacaaggtgttcccacccgaggtcgctgtgtttgagccatcagaagcagagatctcccacacccaaaaggccacactggtgtgcctggccacaggcttcttccctgaccacgtggagctgagctggtgggtgaatgggaaggaggtgcacagtggggtcagcacggacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggctttacctcggtgtcctaccagcaaggggtcctgtctgccaccatcctctatgagatcctgctagggaaggccaccctgtatgctgtgctggtcagcgcccttgtgttgatggcaatggtcaagagaaaggatttc

SEQ ID NO: 13 - DNA, 15 bp, synthetic construct
  source 1..15: mol_type = other DNA
atggggcacagggct

SEQ ID NO: 14 - DNA, 18 bp, synthetic construct
  source 1..18: mol_type = other DNA
tacagctatgagaaactc

SEQ ID NO: 15 - DNA, 30 bp, synthetic construct
  source 1..30: mol_type = other DNA
gccagcagccctcccaatgaaaaactgttt

SEQ ID NO: 16 - protein, 307 aa, synthetic construct
MGCRLLCCAVLCLLGAVPIDTEVTQTPKHLVMGMTNKKSLKCEQHMGHRAMYWYKQKAKKPPELMFVYSYEKLSINESVPSRFSPECPNSSLLNLHLHALQPEDSALYLCASSPPNEKLFFGSGTQLSVLEDLNKVFPPEVAVFEPSEAEISHTQKATLVCLATGFFPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSVSYQQGVLSATILYEILLGKATLYAVLVSALVLMAMVKRKDF

SEQ ID NO: 17 - DNA, 807 bp, synthetic construct
  source 1..807: mol_type = other DNA
atgaagaggatattgggagctctgctggggctcttgagtgcccaggtttgctgtgtgagaggaatacaagtggagcagagtcctccagacctgattctccaggagggagccaattccacgctgcggtgcaatttttctttgcagtggtttcatcaaaacccttggggacagctcagactctgtgaacaattcaacctgttttacaaacagaatggaagattaagcgccacgactgtcgctacggaacgctacagcttattgtacatttccattccctcagggacatcttcccagaccacagactcaggcgtttatttctgttttggcaaagggactcatctaatcatccagctgtgcgaggaggtgctgacggactcaccgccctatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 18 - DNA, 15 bp, synthetic construct
  source 1..15: mol_type = other DNA
gactctgtgaacaat

SEQ ID NO: 19 - DNA, 15 bp, synthetic construct
  source 1..15: mol_type = other DNA
attccctcagggaca

SEQ ID NO: 20 - DNA, 30 bp, synthetic construct
  source 1..30: mol_type = other DNA
gctgtgcgaggaggtgctgacggactcacc

SEQ ID NO: 21 - protein, 269 aa, synthetic construct
MKRILGALLGLLSAQVCCVRGIQVEQSPPDLILQEGANSTLRCNFSDSVNNLQWFHQNPWGQLINLFYIPSGTKQNGRLSATTVATERYSLLYISSSQTTDSGVYFCAVRGGADGLTFGKGTHLIIQPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 22 - protein, 10 aa, synthetic construct
ATFLGSLTWK

SEQ ID NO: 23 - DNA, 912 bp, synthetic construct
  source 1..912: mol_type = other DNA
atgggcacgagcttgctctgctggatggccctgtgcctgctgggggcggaccatgcagacaccggcgtcagccagaatccacgtcacaagattaccaagcgcggccagaacgtgaccttcagatgtgaccccatccctgtattggtaccgccagactcttggacagggccctgagttcggagcacaaccgtcctgacctacctggagaagtcccgcctgttgagtgacaggttttcagccgagcggccgaaaggctccttctcgaccttccagaacgaggctcagctagagatccagcgcactgaacaaggtgattctgccatgtacctgtgctttggtcccgggacccggcctcctcttcccagagctacgagcagtactctcaccgtgacagaggatctgcgcaatgtgacaccccctaaggtgtccctgttcgagccatctaaggccgagatcgccaacaagcagaaggccaccctggtgtgcctggcaaggggcttctttcccgatcacgtggagctgagctggtgggtgaatggcaaggaggtgcactccggcgtgtgcacagaccctcaggcctacaaggagagcaactactcctattgtctgtctagccggctgagagtgtccgccaccttttggcacaaccctaggaatcacttccgctgccaggtgcagtttcacggcctgagcgaggaggataagtggccagagggatccccaaagccagtgacccagaatatctctgccgaggcatggggaagggcagactgtggaatcacatccgcctcttatcagcagggcgtgctgagcgccaccatcctgtacgagatcctgctgggcaaggccacactgtatgccgtgctggtgtctaccctggtggtcatggctatggtgaagagaaagaacagc

SEQ ID NO: 24 - DNA, 15 bp, synthetic construct
  source 1..15: mol_type = other DNA
tcggagcacaaccgc

SEQ ID NO: 25 - DNA, 18 bp, synthetic construct
  source 1..18: mol_type = other DNA
ttccagaacgaggctcag

SEQ ID NO: 26 - DNA, 30 bp, synthetic construct
  source 1..30: mol_type = other DNA
gcctcctcttcccagagctacgagcagtac

SEQ ID NO: 27 - protein, 304 aa, synthetic construct
MGTSLLCWMALCLLGADHADTGVSQNPRHKITKRGQNVTFRCDPISEHNRLYWYRQTLGQGPEFLTYFQNEAQLEKSRLLSDRFSAERPKGSFSTLEIQRTEQGDSAMYLCASSSQSYEQYFGPGTRLTVTEDLRNVTPPKVSLFEPSKAEIANKQKATLVCLARGFFPDHVELSWWVNGKEVHSGVCTDPQAYKESNYSYCLSSRLRVSATFWHNPRNHFRCQVQFHGLSEEDKWPEGSPKPVTQNISAEAWGRADCGITSASYQQGVLSATILYEILLGKATLYAVLVSTLVVMAMVKRKNS

SEQ ID NO: 28 - DNA, 801 bp, synthetic construct
  source 1..801: mol_type = other DNA
atggagactctgctgggtctcctcatcctgtggctacagctgcagtgggtctcgtccaagcaggaggtgacccaaattcctgccgcgctgtccgtgcccgagggcgagaacctggtgctcaactgctccttcaccttgcagtggttccgccaggaccccgggaagggcctgagacagcgccatctacaacccagccttctgcttcagacatcaggccgcctgaatgcaagtttggacaaatcttctggccggtcgaccctgtatatccagagctcccagcgcgaaattgcggcttcccagccgggtgattctgctacctacctgtgcttcggcaccggcactcgcctgcagccgtgctgttcaacacggacaagctgatcggtttttccaatccagaatcccgagcctgccgtgtaccagctgaaggacccccgctcccaggattctaccctgtgcctgttcacagactttgattcccagatcaacgtgcctaagacaatggagtctggcaccttcatcacagacaagtgcgtgctggatatgaaggctatggactccaagtctaacggcgccatcgcctggtctaatcagaccagcttcacatgccaggatatctttaaggagaccaacgccacatacccttcctctgacgtgccatgtgatgccaccctgacagagaagagcttcgagacagacatgaacctgaattttcagaacctgctggtcatcgtgctgcggatcctgctgctgaaggtggccggctttaatctgctgatgaccctgagactgtggagctcctga

SEQ ID NO: 29 - DNA, 18 bp, synthetic construct
  source 1..18: mol_type = other DNA
gacagcgccatctacaac

SEQ ID NO: 30 - DNA, 21 bp, synthetic construct
  source 1..21: mol_type = other DNA
atccagagctcccagcgcgaa

SEQ ID NO: 31 - DNA, 30 bp, synthetic construct
  source 1..30: mol_type = other DNA
gccgtgctgttcaacacggacaagctgatc

SEQ ID NO: 32 - protein, 266 aa, synthetic construct
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVLFNTDKLIFGTGTRLQVFPIQNPEPAVYQLKDPRSQDSTLCLFTDFDSQINVPKTMESGTFITDKCVLDMKAMDSKSNGAIAWSNQTSFTCQDIFKETNATYPSSDVPCDATLTEKSFETDMNLNFQNLLVIVLRILLLKVAGFNLLMTLRLWSS

SEQ ID NO: 33 - protein, 9 aa, synthetic construct
SLLMWITQC

SEQ ID NO: 34 - DNA, 933 bp, synthetic construct
  source 1..933: mol_type = other DNA
atgagcatcggcctcctgtgctgtgcagccttgtctctcctgtgggcaggtccagtgaatgctggtgtcactcagaccccaaaattccaggtcctgaagacaggacagagcatgacactgcagtgtgcccaggatatgtcctggtatcgacaagacccaggcatggggctgaggcatgaaccatgaatactgattcattacactgaccaaggagaagtccccaatggctacaatgtctccagatcaaccacagaggatttcccgctctcagttggtgctggtatcaggctgctgtcggctgctccctcccagacatctgtgtacttctgttttggagaaggctcgccagcagttacgtcgggaacaccggggagctgttttaggctgaccgtactggaggacctgaaaaacgtgttcccacccgaggtcgctgtgtttgagccatcagaagcagagatctcccacacccaaaaggccacactggtatgcctggccacaggcttctaccccgaccacgtggagctgagctggtgggtgaatgggaaggaggtgcacagtggggtcagcacagacccgcagcccctcaaggagcagcccgccctcaatgactccagatactgcctgagcagccgcctgagggtctcggccaccttctggcagaacccccgcaaccacttccgctgtcaagtccagttctacgggctctcggagaatgacgagtggacccaggatagggccaaacccgtcacccagatcgtcagcgccgaggcctggggtagagcagactgtggcttcacctccgagtcttaccagcaaggggtcctgtctgccaccatcctctatgagatcttgctagggaaggccaccttgtatgccgtgctggtcagtgccctcgtgctgatggctatggtcaagagaaaggattccagaggc

SEQ ID NO: 35 - DNA, 15 bp, synthetic construct
  source 1..15: mol_type = other DNA
atgaaccatgaatac

SEQ ID NO: 36 - DNA, 18 bp, synthetic construct
  source 1..18: mol_type = other DNA
tcagttggtgctggtatc

SEQ ID NO: 37 - DNA, 36 bp, synthetic construct
  source 1..36: mol_type = other DNA
gccagcagttacgtcgggaacaccggggagctgttt

SEQ ID NO: 38 - protein, 311 aa, synthetic construct
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 39 - DNA, 825 bp, synthetic construct
  source 1..825: mol_type = other DNA
atgatggagaccctcttgggcctgcttatcctttggctgcagctgcaatgggtgagcagcaaacaggaggtgacgcagattcctgcagctctgagtgtcccagaaggagaaaacttggttctcaactgcagtttcactctccagtggtttaggcaggaccctgggaaaggtcgatagcgctatttacaactcacatctctgttgcttcaaacaagtggaagacttaatgcctcgctggataaatcatcaggacgtagtactttaattcagtcaagtcagagagagtacattgcagcttctcagcctggtgactcagccacctacctctgttttggaagagggctgtgaggcccttatacggaggaagctacatacctacaaaccagccttattgttcatccgtatatccagaaccctgaccctgccgtgtaccagctgagagactctaaatccagtgacaagtctgtctgcctattcaccgattttgattctcaaacaaatgtgtcacaaagtaaggattctgatgtgtatatcacagacaaaactgtgctagacatgaggtctatggacttcaagagcaacagtgctgtggcctggagcaacaaatctgactttgcatgtgcaaacgccttcaacaacagcattattccagaagacaccttcttccccagcccagaaagttcctgtgatgtcaagctggtcgagaaaagctttgaaacagatacgaacctaaactttcaaaacctgtcagtgattgggttccgaatcctcctcctgaaagtggccgggtttaatctgctcatgacgctgcggctgtggtccagc

SEQ ID NO: 40 - DNA, 18 bp, synthetic construct
  source 1..18: mol_type = other DNA
gatagcgctatttacaac

SEQ ID NO: 41 - DNA, 21 bp, synthetic construct
  source 1..21: mol_type = other DNA
attcagtcaagtcagagagag

SEQ ID NO: 42 - DNA, 39 bp, synthetic construct
  source 1..39: mol_type = other DNA
gctgtgaggcccttatacggaggaagctacatacctaca

SEQ ID NO: 43 - protein, 275 aa, synthetic construct
MMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 44 - protein, 9 aa, synthetic construct
SLLMWITQC

SEQ ID NO: 45 - DNA, 1098 bp, synthetic construct
  source 1..1098: mol_type = other DNA
atggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccctgaacgaggacctgcgctcttggaccgcggcggacatggcagctcagatcaccaagcgcaagtgggaggcggtccatgcggcggagcagcggagagtctacctggagggccggtgcgtggacgggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgctgtgatgtggaggaggaagagctcagatagaaaaggagggagttacactcaggctgcaagcagtgacagtgcccagggctctgatgtgtctctcacagcttgtaaagtgtga

SEQ ID NO: 46 - DNA, 1097 bp, synthetic construct
  source 1..1097: mol_type = other DNA
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacggacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatgggagccgtcttcccagcccaccatccccatcgtgggcatcattgctggcctggttctctttggagctgtgatcactggagctgtggtcgctgctgtgatgtggaggaggaagagctcagatagaaaaggagggagctactctcaggctgcaagcagtgacagtgcccagggctctgatgtgtctctcacagcttgtaaagtgta

SEQ ID NO: 47 - DNA, 360 bp, synthetic construct
  source 1..360: mol_type = other DNA
atgtctcgctccgtggccttagctgtgctcgcgctactctctctttctggcctggaggctatccagcgtactccaaagattcaggtttactcacgtcatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtgggatcgagacatgtaa

SEQ ID NO: 48 - DNA, 867 bp, synthetic construct
  source 1..867: mol_type = other DNA
atgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataa

SEQ ID NO: 49 - DNA, 1098 bp, synthetic construct
  source 1..1098: mol_type = other DNA
atggccgtcatggcgccccgaaccctcctcctgctactctcgggggccctggccctgacccagacctgggcgggctcccactccatgaggtatttctacacctccgtgtcccggcccggccgcggggagccccgcttcatcgccgtgggctacgtggacgacacgcagttcgtgcggttcgacagcgacgccgcgagccagaggatggagccgcgggcgccgtggatagagcaggaggggccggagtattgggaccaggagacacggaatgtgaaggcccagtcacagactgaccgagtggacctggggaccctgcgcggctactacaaccagagcgaggacggttctcacaccatccagataatgtatggctgcgacgtggggccggacgggcgcttactccgcgggtaccggcaggacgcctacgacggcaaggattacatcgccctgaacgaggacctgcgctcttggaccgcggcggacatggcagctcagatcaccaagcgcaagtgggaggcggcccatgcggcggagcagcagagagcctacctggagggccggtgcgtggagtggctccgcagatacctggagaacgggaaggagacgctgcagcgcacggacccccccaagacacatatgacccaccaccccatctctgaccatgaggccaccctgaggtgctgggccctgggcttctaccctgcggagatcacactgacctggcagcgggatggggaggaccagacccaggacacggagctcgtggagaccaggcctgcaggggatggaaccttccagaagtgggcggctgtggtggtgccttctggagaggagcagagatacacctgccatgtgcagcatgagggtctgcccaagcccctcaccctgagatgggagctgtcttcccagcccaccatccccatcgtgggcatcattgctggcctggttctccttggagctgtgatcactggagctgtggtcgctgccgtgatgtggaggaggaagagctcagatagaaaaggagggagttacactcaggctgcaagcagtgacagtgcccagggctctgatgtgtctctcacagcttgtaaagtgtga